SEQUENCE LISTING (76 sequences)

SEQ ID NO: 1 - RNA, 1569 nt, Lophophora williamsii
  source 1..1569: mol_type = mRNA
atgggttcccttgatatcaaacaagaatcatcacccctcatgacaaaccctctagactcagaggagttccgtaggcaagggtacatggtcatagacttcctcgcagagtattacaagaacatccagaagttcccagttagaagccaggtggagcccggctaccttcgaaagaggctaccagagtccgccccatatgaacctgagtctattgagaggatcctcaaagacgtccatgatgacatcgttccaggcctgacccactggcagagtcctaattactatgcatacttccctagcagtggcagcacagctggtttacttggagagaccttagctgccgggttcaatgtggttgggttcaactggatatcctcaccagcatctaccgagctagagagcatcgtcatggactggcttgctgagatgctcaacttgcccaagtcattcacattctcaggagatggtggtggggttatgatgggaaccacctgcgaagccatcctaaccaccataaccgcagcacgtgatcgaatcctcgatcgcattggacgcgaacacatcaacaagcttgtcgtatacggctccgatcaaacccactgctccttcttcaaatcagccaagatagccgggattttgccaaataacttccgccaggtgaagacttcaagggtcaatgcattttcgatgagaccagatgcactaagggcagcaatccaagccgacgtcgacgcaggtttggtcccattctttctctgcaccaccgtggggaccacatcgacggctgcagtagacccggtggcgctcctctgtgaggtggccaaagattacggaatgtgggtgcacatagatgccgcctatgctggaaacgcatgcatatgccccgagttcaggcacatgatcaatggggttgagaacgctgattcgttcagcttcaacactcataagtggttcctaaccactctagattgctgctgcctttgggttaaagatccaagctcccttgttaggtgtctctcaacaaaccctgagtacttgaagaacaaggcaactgacacccaacaggtagtcgactacaaggactggcagatcaccctgagccggcggttcaggtccctgaagatgtggctcgtgctcagaagctacggagtcgactacctccggaacttcttgaggagccacgtcgacatggctgcttactttgagcagttggttcggatggatggaaggttcgaggtggtggtcccaaggagctttgcacttgtgtgcttcaggctttcggcctcagcggtgattcagcgtttgtatgatagctgccaccgacatggccacggacagaagttccttgaggaggagaccctcaatgatgtaaacgcagagctcctggagtccgtcaactctgccgggcagatctacatgactcactcccttgtcgatggcgtttacatgctgcggttcgcggttggagccaccctcactcaaacccaccacgtcacatatgcttggaagcaggttcaagatcatgcaagtgcagttttagcacgcaggattaatagcatctgcaactgctaa

SEQ ID NO: 2 - RNA, 1293 nt, Lophophora williamsii
  source 1..1293: mol_type = mRNA
atgcttagtgccgggattaacattgttggctttagctggatgagctgtcctgcggcaacagaactggaaatcattgttttagattggcttgccaagctgctgaatctacctgataccttcctatccactgggaacggtggaggtgtcatacagggcactgcaagtgaagctgttctagtcgcattacttgctgcccgtgataagaagttgaaaaagattgggaaagattccgtggggaaacttgttgtgtattcatcagatcaaacacattcttctgtgcaaaaagcctgtcaaattgcaggtattcacccagagaactttaggatactgaagacggactcttcaactgaatatgccctttctccaggagtagttttgaatgccatatcgaaagacgttgcttctggccttatacctcttcttctgtgcgcaactgttggtaccacttcgtcaacggcaattgatccattggttgatcttggaaagatagctaagagtaatcgaatttggttgcatgttgatgctgcttatgctggaagtgcttgcatatgccctgagtatcgccactacatcaacggtgttgaagaagcagattcatttaacatgaatgcacacaaatggcttcttacaaattttgattgttcagctctctggattaaggacagaaatgctctcatacaatccttgtcgacatatccagagtatttaaggaacaaagcttcagaggctaacgtggtggtagattacaaggactggcaaattcccctcggccgtcgatttaggtcattgaaactatggatggttctgaggttatatggccaggagggcctgcaatcgtacatacgaaatcatatagccttggccaaacagttcgaagagcttgttattcaggattctagatttgagatggtcactcccaggaggttctcattagtgtgcttccgcttgttgcctcgttctacagatgaaaaccaagctgataagctcaaccgtgctctcttggatgccgtcaacttaactggagccatattcatttctcacacggtgctgtcagggatgtacgtgctccgtttagctgttggagctccattgacagaagagcggcatgtaattgcagcatggaaggtcttgcaagaaaaggccaccgcccttctcgagggtaacgtggcccaagagcccaatggacatgctcagctgtcaaacggagtcattgagttggacggtgcgctggacaatggggtgacaacggagcagcaggggcatgtcgatgatctgccaattaagtcaaagtga

SEQ ID NO: 3 - RNA, 1593 nt, Lophophora williamsii
  source 1..1593: mol_type = mRNA
atggacgggcaaatgttgaagccaatggatgcagagcaactgagggagtatggccatcagatggttgattttgttgctgattactacaagaccatcgagagttttcctgttcttagccaagttcagccagggtatcttcgtgaacttgtaccaaccaatccacctactcatcccgaatctctacaagatgtccttgatgatgtaaaagcaaaaatattgcagggtgtaactcattggcaaagcccaggctattttgcatattttccttgcaatagcagtactgcagggttcctcggagaactgctgagtgctggtattaacattgttggctttagctggatgagctgtcctgcggcaacagaactggaagtcattgttttagattggcttgccaagctgctgaatctacctgattcgttcctttctattgggaatggtggaggtgtcatacagggcactgcaagtgaagctgttctagtcgcattacttgctgctcgtgataagaagttgaaaaagatcgggaaagattccatgaggaaactggttgtgtattcatcagatcaaacacattcttctgtgcaaaaagcctgtcaaattgcaggtattcacccagagaactttaggatactgaagacagactcttcaactgaatatgccctttctccagaagtagttttgaatgccatattgaaagatgttgcttctggccttgtacctctttttctgtgcgcaactgttggtaccacttcgtcaacggcaattgatccattgcttgctcttgggaagatagctaagagtaatcgaatttggttgcatgttgatgctgcttatgctggaagtgcttgcatatgccctgagtatcgccactacatcaacggtgttgaagaagcagattcgtttaacatgaatgcacataaatggctccttgcaaattttgattgttcagctctctggattaaggacagaaatgctctcatacaatccttgtcgacatatccagagtatttaaggaacaaagcttcagaggctaacgtggtggtagactacaaggactggcaaattcccctcggccgtcgatttaggtcattgaaactatggatggttctgaggttatatggccaggagggcctgcaatcatacataagaaatcatatagccttggccaaacagttcgaagagcttgttattcaagattccagatttgagatggtcactccgaggaggttctcattagtgtgcttccgcttgttgcctcgttctacagatgaaaaccaagctgataagctcaaccgtgctctcttggatgccgtcaacttagctggaaccgtgttcatttctcacacggttctgtcggggaagtacgtgctccgtttggctgttggagctccattgacagaagagcggcatgtaattgcagcatggaaggtcttgcaagaaaaggccaccgcccttctcgagggtaacgtggcccaagagcccaatggccatgctcagctgtcgaacggagtcgttgaggtggatggtgcgctggacaatggggtgacaacggagcagcatggccacgtcgatgatctgccaattaagtcaaagtga

SEQ ID NO: 4 - RNA, 1506 nt, Lophophora williamsii
  source 1..1506: mol_type = mRNA
atggcgctggtgctcctcctagttgtagtttgctctttcatcttcttccttgtaattctcccacgaaaacaaggaaaattgaatctccctcctggacccaaaggcctcccccttatcggaaacctccaccagttcgattccttggcccctcatctctatttctccaagcttgccaagatctacggcccgatcttgtccttgcgattcgggtgtagatcagcggtggtgattcaatccgctgctctagcgaaggaggtcctgaaagcacaggatcacaacttctgtacgaggccaactctggtggctcagcagagactgtcttacaatggttcagacattatttttgcgccctacaatgagtgtttcagagagcttaggaaattgtgcgttgttcatttgtttagctccaagaaggttcagtcttccacccccattcgacaagaagaagtttctagaatgatcaagaagatcactgctctgtcttctgcttcaaaagtcgttaacttgagcaacttgctcatggctcttaccagctccattatttgcagggttgcttttggcaaaagatatgagtatgaaggcgttgaagggagtagatttcatgaagtcctgaacgaagttcaagctattgcggcagctcctttcttcacagattatttccctttcataggaagctggcttgataagctaaccgggctgtcatgcaggcttgataaggtgtttaagaaaatggataaattttacgatgaaatcatcaatgatcaccttgatcccaataggcctaaagatgacagtcatgaggatatcattgatgttcttctgcagctccggaaggagcgttgcttctcttttgagctcaccttggaccacatcaaagcagtattaatggatataattgtagcaggaactgatacaagtgcagcaattgtagtttggacaatgacggagttaatgaaacatccaactatgatgatgaaagttcaagaagagcttagaactgtggttcaagacaagacatttatcaatgagagtgatctctcaaagttgacgtacctcaaagctacagtgaaagaaacacttcgactgcacaatgctggtccattattggttgtccgggaagtaataaaaaaatgtagcatacaagggtatgatattctcccaaaaagcttggtatttgttagcatatgggaaatcggtagagaccccaagttttggactgatcctgaaatattcatgcctgaaagatttcttgggagctcaattgattttaaagggcaagattttgaactgataccattcggagcaggtagaagaatatgtcctggaatgcttcttggtgttgctaatgtggagcttgcactagccaatttgttgtacactttcgagtggaagttgccagttggaatgacggaggaagacatcggcttcgatgtaatacccggaatcacaatgcacaaaaagaatccactgtgccttgtagcaaagaagttttctcatgtaaactaa

SEQ ID NO: 5 - RNA, 1572 nt, Lophophora williamsii
  source 1..1572: mol_type = mRNA
atgcatgagcttttagctgcatccatggactccatacggggcagcccgaccctgatgggagtcttgttattcctaggtccgatcctccttctcgggctgatatcgaggctaaggcgaaaaccctacccgcccgggccgaaggggctgcccatcataggcaacatgatgatgatgggccagctgacccaccgtgggttagccaagttggctcagcgctacggcggcctgttccacctgaagatggggctcgtccacatggtggtagtttcggggcccgaagaggcccgtcaagtcctccaagtccaagacaacatcttctcgaaccggcccgccagcatcgccatcagctacctcacctacgaccgggccgacatggccttcgcccactacgggcccttctggcgccagatgcgtaagctctgcgtcatgcgcctcttcagccgcaagcgggccgagtcgtggcagtccgtccgcgacgaggtcgaccacatcgtccgggccatggccacccaggccgggtcccccgtcaacgtgggcgagctcgtcttcaacctcaccaagaacgtcacataccgggccgcattcgggtcgaactcgtccgagggccaggacgagtttattgccatactccaagagttctcgaagctgtttggggcgtttaatattgcggatttcatcccgtttcttacatgggtcgacccaaacggactcaacgaccgactcgccaaggcccgaggagccctcgataggttcattgataagattattgatgatcatatggagaaaaagaaggcaaatggtcatattaagggtggtggtgtgaaagaagaggaggatatggtggatgatttgttgaatttttacagcgaggaagccaaagttactgagtctgaggatttacagaacgccatcaagcttactagggataacatcaaagctatcatcatggatgtgatgtttggtggaactgagacagtggcctcagcaattgagtgggccatggcagaactcatgcactgccccgaagacatgaaacgggtccaacaggagcttgccgatgttgtgggtctgacccgccgggtccaggagtcggatctcgaccagctcccatttttcaggtgttgcatcaaagagactctccggcttcacccgcccatccctcttcttctccatgagaccgccgagccggccatgatcggagggtaccaaatacccaaacgggcccgggtcatgatcaatgcatgggctattgggcgtgacccaaactcgtggtccgaggcggagacgttcaggccaagccggttcctccaagatggggtgcccgatttcaaaggcggcaacttcgagttcatcccattcggatcgggtcggaggtcgtgcccgggtatggcgcttggcctgtacgcgctggagatgtgcgtggcaaatcttctacactgcttcaagtgggaactaccagatgggatgaaaccgagtgagctcgacatgagtgacacattcggactcaccgctccgagggcgagtcgactcgtcgcggtgccgagtccacggctgttgtgcccgctcaactaa

SEQ ID NO: 6 - RNA, 1602 nt, Lophophora williamsii
  source 1..1602: mol_type = mRNA
atgcaacaattacactcattcataggctcactccttgtgtcatccaaagatgacccaaccaaaatggtctcaaggctactcttcctcttcatagcccctgtggcggccctcctcctcatcgtccggcgggccaggcggaaaccatacccgccgggcccaacgggtctgcctctaatagggaacatgatgatgatggaccaactaacccaccggggcctggcccgcttagccaagcgttacggcgggctcttccacctaaagatggggttcgtccacatgatcgtggtctcgagcccggacgaggcccgacaagtcctccaggcccaggaccatattttctcgaaccgacccgccaccatcgccatcagctacctcacctacgaccgggccgacatggccttcgcccactacggcccgttctggcgccagatgcgcaagctctgcgtcatgcgcctctttagccgaaaacgggctgagtcgtggcagtcggtccgtgacgagaccgaccgtatcgtccgggccatggcggcccggactggctcgcctgttaacgtcggcgagcacgtgttcaacctcaccaagaacgtcatttaccgggccgcgtttgggtcgagctcgtccgagggtcaagaggagttcattgccatacttcaggagttctcgaagctgttcggagcgtttaatattgcggattttataccgtttctttggtgggtcgacccaaatgggctgaacagtcggctcgtcaaggctcgggccgccttggaccggttcatcgataatattattgacgagcatataagttataagcagaagaagccaaacggcgtcgtcaaggaggaggcggatatggtggatgatttgctcaacttttatagtggtgatgaaatggggaaagttagtgagtcggatgatcttcaaaatgctatcaaactcactagggataatatcaaagccatcatcatggatgtaatgtttggtggtaccgagaccgtagcctccgcaattgaatgggccatggcagagctaatccggagccccgatgacctcaaacgggttcagaaagagctcgaagacgtagtcggcctgacccgtctggttgatgaatcggacctcgacaagctcacttactttaggtgctgcattaaggagaccctccgccttcacccgcccatccccctactcctccatgagacggcccaggaggcggtcatcagcgggtatcggatcccaaagcaagcccgggtcatgatcaacgcgttcgcgatcggacgtgatccgaactcgtggtccgacccggattcgttcaagcctggccggttcttaaatgagggtgtgcccgacttcaagggtggcaacttcgagttcatcccattcgggtcgggccggaggtcatgcccgggaatgcagcttgggctgtacgcgcttgagatgtgtgtggccaacttgctacactgcttcacgtgggacctaccagatgggatgaaaccgagtgaactcagcatgggcgacatattcggactcaccgcgccaagggcgacccgactcattgcagtccccactccacgtcttttgtgcccgctttacgaacacgcgccgtga

SEQ ID NO: 7 - RNA, 1590 nt, Lophophora williamsii
  source 1..1590: mol_type = mRNA
atgcaacaattacactcattcataggctcactccttgtgtcatccaaagatgacccaaccaaaatgttctcaagactactcttcctcttcatagcccctgtggcggccctcctcctcatcgtccggcgggccaggcggaaaccatacccgccgggcccaacgggtctgcctctaatagggaacatgatgatgatggaccagctaacccaccggggcctggcccgcctagccaagcgttacggcgggctcttccacctaaagatggggttcgtccacatgatcgtggtctcgagcccggacgaggcccgacaagtcctccaggcccaggaccatattttctcgaaccgacctgccaccatcgccatcagctacctcacctacgaccgggccgacatggccttcgcccactacggcccgttctggcgccagatgcgcaagctctgcgtcatgcgcctctttagccgaaaacgggctgagtcgtggcagtcggtccgtgacgagaccgaccgtatcgtccgggccatggcgacccggaccggctcgcctgtcaacgtcggcgagcacgtgttcaacctcaccaagaacgtcatttacagggccgcgtttgggtcgagctcgtccgagggtcaagaggagttcattgccatacttcaggagttctcgaagctgttcggagcgtttaatatcgcggatttcataccgtttctttggtgggtcgaccccaatgggctgaacagccggctcgtcaaggctcgggccgccttggaccggttcatcgataatatccttgatgagcacataagttataagaagaagaggccaaacggcgtcgtcaaggaggaggcggatatggtggatgatttgctcaacttttatagtggtgatgaaatggggaaagttagtgagtcggatgatcttcaaaatgctatcaaactcactagggataatatcaaagccatcatcatggatgtaatgtttggtggtaccgagaccgtagcctccgcaattgaatgggccatggcagagctaatccggagccccgatgacctcaaacgggttcagaaagagctcgaagacgtagtcggcctgacccgtctggttgatgaatcggacctcgacaagctcacttactttaggtgctgcattaaggagaccctccgccttcacccgcccatccccctactcctccatgagacggcccaggaggcggtcatcagcgggtatcggatcccaaagcaagcccgggtcatgatcaacgcgttcgcgatcggacgtgatccgaactcgtggtccgacccggattcgttcaagcctggccggttcttaaatgagggtgtgcccgacttcaagggtggcaacttcgagttcatcccattcgggtcgggccggaggtcatgcccgggaatgcagcttgggctgtacgcgcttgagatgtgtgtggccaacttgctacactgcttcacgtgggacctaccagatgggatgaaaccgagtgaactcagcatgggcgacatattcggactcaccgcgccaagggcgacccgactcattgcagtccccactccacgtcttttgtgcccgctttactaa

SEQ ID NO: 8 - RNA, 1503 nt, Lophophora williamsii
  source 1..1503: mol_type = mRNA
atggatatggagatagcatgctgtttcgtagcagttttggcctccattttcttcctctgtaagctcctttttgcggctaagagcaagcagaggaacaagaaaagtttgccgccaagcccaccaacgatccccatattgggccacctccaccttctcaagccacccttccaccggaccctccacgccctggcccaacgctacggcccaatcttctatctccgcctcggcagccagcctgccgtggtggtctccgccccatggaccgccgaggagtgcttcacacgaaacgacgtcgtcttcgccgaccggcccaagttcctcgtgggccaccacttgggctacaacaactccatcctgctgtggtctccctatggcccccactggcggaacctccgacgtctcgccaccataaccgccctatctcaaaagcagatcaacctcatgggccccgcccgtcgggtcgaggtccgaaacatgatccgagacctatttcggagctccgaaagcggcacccggaacgtgaacctgaatgaggcgtttgccaagctgacaaggaaccttgtgatgagggcagtgaatgggagaccgtgggagactacgataatgaccccgccttcgcaattgatgactgtgtgtgatttcgtgcccgttttgagatgggccgggtataaggggatagagaaggagatgataaagctgaggaaagagagagacggggtactacagagtctgcttgatgagtatagagaaagccgagcaattcgtcgaacaggtggtgtggatggtgatgacgcggagaaaaaaacaatgatggacgacttgcttgatttgcaagaggctgagcctgaatattatactgatgacacgttgaaaggactcgttctggttatgttacttgcgggatcagatacttcagctcgaactttggagtgggcaatggcgcttcttctgaaccacccagaagtcttggagaaggccaaaaccgagattgacacacacatcgacaagggtcgcatagtggaagactctgatcttcctagactaacctacataagttgcattatcaacgagacactaaggcttttcccaccggcacctcttctagttccacattactcgtcccaagattgcaccataggaggataccatgtacccaagggcactttgctcctagtcaatgcttgggcaatccatagggaccctgcgctctgggatgagcctaccaagttcaagccagagcgattcgaaaaggacaaattggaagggttcagctttaagtattttccctttggaatgggaaggagggcttgtccagggaccaattttgccctaaggaatgtgacattggtgttagccactcttcttcagtgcttcgattggaaagtagctgagcctgtggacttgaacgagcgagccggtgccatcatcatgcctaaggaaaaacccctccatgcaatctgccaccctcggccttccatgatgggcactctctctcaaatctga

SEQ ID NO: 9 - RNA, 1530 nt, Lophophora williamsii
  source 1..1530: mol_type = mRNA
atggaagagctacaattgttcagttgtgcaatcatcttgttggttatcgtcatagctcataagttgttcaaaaaacccaaacatcaagagagaaatctgccaccaagtcccccatctctgcctatcatagggcacctacacatgctaaaagagcccctacacaagaccatccacgacatctccaccaagtacggcgaagtcctattactcaagcttggtgttcgccgagccctcatcatcgcctccccctcggctgtggaagaatgcctactgaagaacgacaccacctttgccaaccggccggacacccttgtagggcggctcctccactacaacagcacctccatagccttctccccctacaacggccattaccggaccctcagacgagtcatgacccaggaagttttctcctctaccaaagtccctcttttttctggcatccgtcaggccgaggtccgcctcctcctgagggacatctgggaagaatgcactaccactggtgatgttgacacgaagacaatccatttgaacagagggttcaatgagcttgccttgaatatcatgaccaacatggtggctgggaagaggtattatgggaaggatgtggaggatgtcaccgaagctaagaggttcagcgagattataagggaaggagcggtattgcatggggcggcgaatcctggagatcttttcccgatgttaaagtggatgtttaggggtttggagaagaggatggtcacatggatgaagagcactgacgcgttctaccaaaagctgctcgatgaacgaaagcagacgatgagcaaggataatactagggctattgttgatgttttgatcaagctacagcagcaagatcctgcgttttttactaatgaagtcatcaagggcatgatcatggtattgatactcgctggagcagacacttcgtccgccacaatggaatggtcaatggcactcctcttaaacaatccccactgcctaaagaaggcgcaggaagagatagacacggtgatagggcacaaccgacttataaacgaagaggatgtcgcaaagctaccttacctccaaaacatcgtcatggagacactccgtttgtacccaccaacaccgcttctcctgccccacgagacctcagaggactgcaacatctgtggctacgacattccgcaaggaaccatgttgatggttagtttatggactctccaccgcgacccaagatggtggaatgagcctgaaaagtttattccggagagatttgaagggaaggaaggagaatcgctttataagcttttaccctttggtctgggacggaggaattgccctggttatgcaatgggcaagagagctgtcgctttggttttagggtctgtgcttcaatgctttgactggagaaggccaagtgatgaaatgcttaatatgtcacaatccagcgggattaccatgcctaaagcacagcccttggaagccttctgcacaatccgcccttgtatggctgatttgctctcaaaagaactcacctgttag

SEQ ID NO: 10 - RNA, 1503 nt, Lophophora williamsii
  source 1..1503: mol_type = mRNA
atggattacctgagtttcctgctatgccttgcgatggcattggttgctatgcacttcctactatcaatggcaaggaaaggcaaagagtcgaatccaagacgactgccacccggtccatcccctctacccatcgttgggaacctcttcagccttggaagcaagccccatgtgtccttgactgaacttgccaagacctacggccctcttatgatcctccaacttgggtgggtgccaactgttatcatctcttcagctgctgttgccaaagaagcccttcaaaagaatgatctttccttctccaaccgcacagtccttgatgctgcttgtgcactcgaccaccatcaaaattcaatagcttttttgccagcagcatctagatggcgcaatttccgtaagatctgcaactcccacgtcttttctaccagtaggctcgacgcaagccaatgccttcggagaaataaggtaaaagatctcctttcctatgtcgaaaagtgcagcaaagatggagtcgtagtggatgttggccaactagccttcgtcaccaccctcaatctgttatcaaacactttcttctcagtggatttggctgacttcagctctgaatctgctcatcaatttaaagagattgtacggagtgtgctggaagaagtgggaaagcccaatgttgcagattatttccctctcctcagaaggatggatccacagggcatcaggcagcgcgtaggtgttcattttcaaaagatgatcgatacacttaacagcatgatcaaccgacggctcgagggaacgaggccatcaacttcgttgcaagctcatgatgttctggatgcattgttagacatcagtcatgacaagactgaagacattgagcaatcacatatccctcacctactactggacctatttgttgcagggacggacacaacatcaagcaccctcgaatgggcaatggcagaactgctccgcagcccagataaaatgaagcttgctcaagcagagctagaagaaataattgggaaaggaaaccccgttgaagaaccagacattgtgcgactcccatatttgcaggcagttgttaaggaaaccctcagattacacccagcagtgccatttctagttcccagaaaagctgattcagatgtgagtctcagtggctttacagtgcccaaaaatgcacaagtgttggtcaatgtgtgggcaattgggaaagatccggaaagctggcacaaccccagttcatttcagccagagagatttctaggttcagagtgtgacgtgaagggtcgtgactttgagttgatcccatttggcgcaggccgtcggatctgccctggtttgccattggcaattcggatgatccctctgatgctgggttcactgattcacgagtttaattgggagggggagggtcagactccaccagagatgatggatatggaggagagatttgggttcaccttggtgagagctcaacatcttcgagctattcctttacgtctctga

SEQ ID NO: 11 - RNA, 1527 nt, Lophophora williamsii
  source 1..1527: mol_type = mRNA
atggagacttggtttctgatcatcgtcacagcttgcctaggagctttactcaaagcgttcctctctcttaaacccaaaccaaaactgcctccagggccaggtggggtgcccatattcaacagcatcaaatggctgggggcatcctccaccgacatcgaatcctacctcagaaacctctgtcaaaaatttggtccaattatcaccatacacatgaacacacgcccagcgatctttgtgtcaagccacaccatagcccaccaggccttggttcagaatggcgccatcttttctgaccggcctaagccaactgccacaggtaagattgccactgccaaccagcacaacatcagctccgctgggtacggacccacctggcgcaccctacgtcgcaacctaacttcccaaatcctccacccttcccggcttaaggaatactcccatgcccgtaagtgggtactcgatatcctaatcacccgcttcggctctgatggtgatgatgctgtcaaggtcgttgatcattttcagtttgctatgttttgcttgctggttcttatgtgttttggagacaagcttgaggaatcaaagattaaagaggttgaggacgttcagagatcgttgctggtgtcgttcactaaatttcaggtgcttaacttcttccctagattgagccggcttctgttctggaaacgttgggagttgttcttcaggttgaggaagaggcagcaggaagtgctaattcctttaatcagagcgcgtaaggagattaacgaacaagatggggagaagccagtcacctcctatgtggatactctattgaatttggagattcaggatgatgatagtggaagtaagagaaagctgacggaagatgaattgatcagtgcttgctcggaatttcttagtgcagggactgacaccacctcaacggctttacagtggattatggctaatctggttaagtacccagaggttcagactaggctttttgctgaaatcagaggagttattggagctgaagcggagcatgttggagaggaggacttgcctaagatgccttatcttaaggcagttgtcttggagggcctgaggcggcacccaccaggtcactttgtgttgccgcatacagtgatccaagaagcggacttgggtgggtatgtcgtgcccaagaatgcagtgatcaatttcatggtggcagagatgggttgggatccacaggtatgggaagacccgttggagtttaagcctgagagatttctgcctgctaatggaagcgatgtggagtttgatatcactggtagccgtgaaataaagatgatgcctttcggtgctggtaggaggatctgtccaggttttggcttggccattctgcatctggaatactttgtggccaatctggtctggaagtttgaatgggttgcggccaagggtcatgaggtagatctgtctgagaagcaagagttcaccatcgtgatgaaacatccactccatgctcgcatttctcctaggcgtctttcatcaagtaattga

SEQ ID NO: 12 - RNA, 1515 nt, Lophophora williamsii
  source 1..1515: mol_type = mRNA
atggatctagtgttgttacagaagatcctatatgctctgttctttgctattacggtagcgattgtggtgtcgaaattgaaggggaagaagttgaagctcccaccaggcccattgcccgttcccgtgtttgggaactggctacaggttggagatgatctcaaccacaggaacctgacggagctagccaagcggttcggcgacattttgttgctcaggatgggccagcgcaacctcgtcgtcgtgtcctccccggagctagccaaggaggtgctccacacccagggagtggagttcgggtccaggacccgaaacgtcgtcttcgatatcttcaccggtaagggccaggacatggtgttcaccgtctacggcgagcactggcgtaagatgaggcggatcatgaccgtccccttcttcaccaacaaggtggtccagcaatacagaagcgggtgggaagccgaggcagcggccgtggtggcggacgtgaagaagaaccctgagtccgccacgaccgggatcgtcctccgccggcggctccaactgatgatgtacaacaacatgtaccgcatcatgttcgaccgccggttcgagagcgaggacgatccgctgttcaacaagctcagggctctcaacggtgagaggagtcgattggctcagagcttcgagtacaactatggcgattttatccccatcctaagaccttttctcaggggttacctcaagatttgccgggaggtcaaggagaggcggttgcagctcttcaaggattattttgtcgatgagcgcaagaaattggccagcacaaagccaacaagcaacaatgagctgaaatgcgccattgatcacatcttggacgcacaacagaagggagagatcaatgaagacaatgttctttacatcgttgaaaacatcaatgttgctgcaattgagacaacattgtggtcgatagaatgggcaattgcagagttagtaaaccatccacacattcaaaacaagttgagggaagaactagacacagtgctgggcaaagggcaccaaatcacagagccagacacccacaagttaccatacttgcaagcagtgatcaaggagacactaagactacgtatggccatcccactgctcgttccacacatgaacctccacgatgccaagctgaacggctatgacatccccgcggagagcaagatattggtgaacgcgtggtggctagcaaacaaccctgctcactggaagaaccccgaggagttcaggccggagaggttcttcgaggaggaatccaaggtagaagccaatggcaatgactttaggtaccttcccttcggagttggcagaaggagctgtcctggcattatccttgcactaccaatcctcggcatcaccattggccgtttggtccaaaactttgagatgttaccccctccgggacagtccaagcttgacaccaccgagaagggcggacagttcagtcttcacatccttaaacactccaccattgtcctcaagccgagggactaa

SEQ ID NO: 13 - RNA, 1554 nt, Lophophora williamsii
  source 1..1554: mol_type = mRNA
atggattaccctgctctgttcttctacattttcaccttcctcctctcttacatactcacccaaacactcctgcgaaagttccagaaccttccgccctctcccttcccaacactccccatcctgggccatctctacctcctccggcaccccagctcagcccaccggactttggccaatctctcggcccattacggcccaatcctcttcctccagctcggggctcgccccgccctcctcgtctcctccccttcggccgccgaggaatgcctgggccgcaacgacgtcgtcttcgccaaccggccccgcctcgtcggggcaaaaatcctgggctacgagtacaccaacatcctgtgggccccctacggcccgttgtggcgcacccaccgtcggatcgccgcgaatgaaatcctgtctcctcatcggctccaactgctgtcgtggatccgggccgacgaagtccgatcgttgattcgccggatcatgagactttcaggcgatcaggatgggacggtggagattaagccggcgctgtttgaactgacgaacaacgtgatgatgaggatgattgccgggaagaggttttttggggaaggttcagaaacgtcggaggtggcgaaacgtttcaaggagatgttgagagagttgatgggcttaggtggggcgatgacgggtgcagatttgttcccatatttgaggccgttggggttgttcaaagggacagagagaaagatcaaggaggtgtttgggaagatggataggattttccaggatttggtggatgaaaagaggacgaaaattatggaagggggaactgacgagaatggtggtggtaacgaggaaaagagcaagacgttgattgaggtcttgctgcaactacagaaatctgaccctacttactcttctgacaccatagtcaaaggcctcgtccagatattgctgctagcagggtctgagacttctgcttctacaatagaatgggcattatcccttctggtaaacaatccccaagtcctgaccaaggcacaaaaagagattgatgagcacataggatttgatcgtctcctagaggaaaacgacttgaaccatcttcgctacctccattgcatcataaatgagacacttcggatgtaccctgctgctcccttgttgccacctcatgaatcttcagctgattgcgttgtgggagggtatcgcattcctcgagccgccatgctcatggtgaatgtatgggccatacaaaacgaccctaagatttgggatgagccaagtgtattcaagccggagaggttttgggacctcgaaggtgagcataaacttgggtgtaagttcatgcccttcggctcagggagacgggcatgcccaggagaaaacttggctatgcgggttgttggattggctttgggctcgcttatacagtgttttgattggaagagagttggggaagaaatggtggacatggaagagaagcttggagttggcatgtggaaggccaagcccttgcaagtaaaatgtaggcctcgtcaacagatctacaaccttatttcttccatttga

SEQ ID NO: 14 - RNA, 1503 nt, Lophophora williamsii; identical to SEQ ID NO: 10
  source 1..1503: mol_type = mRNA

SEQ ID NO: 15 - RNA, 1506 nt, Lophophora williamsii
  source 1..1506: mol_type = mRNA
atgggctactacgcaattttcgcagttgtgcttcccttcttgtggacttgtttctacttgctaaaactcaacccaacctcaaaactcacgtcaaccatccgcctcccacctgggcctagacctctcccgatcatcggaaacctccaccaactcggcgagaggcctcaccgcaccttggctaacctagccaaggtgtacggccccataatatccctaaagttcgggagcataaccaccattgcggtatcctctgcggatgtagccaaagaaatgtttcaaaaacacgaccttgccctggccagcaggaaggtccctgcagcggtcagggcaaatgggcacgataatttctccgttgcctggctgcctgtgacccccaaatggcggtttctcaggaaaatctccgccatccaattgttctccactcaacgactcgatgctaggcagtcactccgccaggctaaggtagttgagctccttgactacatcaaggcgcgctctaatgcaggagaaccagttgacattggagaggccgcgttcacaacttcacttaacttgttatcaaacacatttttttcaatggatttggctagttatagttcagctgcatcaggggagtttaaagagcttgtttggaagataatggaggagattgggaagccgaatttggcggattgttttcctcttgtagggttcataagcaggatgagtgtgaaccgggagttgatggggtatggaaataagttgaatgaggtgtttgcagaaatcatagagaagagactaagtgcagcgaattcatctgaaggtcgtggcaatggtgatgttcttgataccctacttagaatcatggaagaagatgattctgagctcagtctggatgatatcatgcatcttctcatggacttctttacagccgggacagacacgacctcgagcaccttagaatgggcaatgacggagctactacataatcccgaaaaaatggcaaaagctcaagctgagctcgaacaggtccttggtaaggacactgtgtcgattcaagaatctgacatcgcaaaattgctttacttgcaagctacagtgaaagagaccttaagaatgcacccacctaccgtatttctgttgcctcgaaaggcagactccgatatagacctgtatggtcatttggtacccaagaatgcccaagtctttgtcaatctttgggcaattagctacgacccgagtacttgggagaacccggatttgttctcacccgagaggttcttggatcaagatatcgatatgaagggccaagatttcgggttcataccatttggagcaggaaggagaatatgtcctgggctgacattggcatataggatgttaaaccttatgctgggaactctaattcatgtcttcaattggaagcttggagatgggttgagtcgggaagatttggacatgactgacaaatttgggattaccattcaaaaggctaagcctctccgggctattccaattctgaagtga

SEQ ID NO: 16 - RNA, 1068 nt, Lophophora williamsii
  source 1..1068: mol_type = mRNA
atggggaggttgctgcagatgccatatgaaatgtctgtaaagataatggtgggttctttggagaagaactggttgcccgatgctgtgattaggaggctcactcgccttcttttagcctctcgtcttcgttctggctacaaactcacctctcaacttcagctgtctgacctccttcacttcgtccactctttaagagaaatgccaatagctattcagactgataagccaaaatcccaacattatgagctccctacttccttcttcaagcttgtgctaggaaagcatatgaagtacagttgctgttatttttctgatgaggtgaccaacctagatgctgcagagaaagcaatgttaaatctgtactgcgagagggcacagttaaaagatggccatgctgttcttgatgttggatgtggctgggggtcacttgtcttgtacgttgctcagaattacagcaattgtagggtcactgggatttgcaattcagtgacacagaaagcattcattgaggagcggtgtagggatcttgagctgcagaatgtggagataaaattagcggacattagtaccttcgaaatggagggatcctatgaccgggtattctctattgaaatgtttgagcatatgaagaactatcaggatcttctcaagaagatatccaactggatgaaacctgatggccttctatttatccattacttctgccacaaagcatttgcgtatcactttgaggatgttaatgatgatgattggatcacgagatacttctttactggcgggacaatgccttctgcaaacctcctactgtactttcaggatgatgtctcggttgtcaatcattggcttgtggatggtaagcattatgcacaaacaagcgaagagtggcttaaacggatggacagtaacttgggttcaataaggccaataatggagtcaacttatgggaaggactgcgctaccaagtggacagtatattggagaaccttcttcatagcagtggctgagctgtttgggtacaacaatggcgatgaatggatggtggctcatttcctgtttaagaagaaatga

SEQ ID NO: 17 - RNA, 1095 nt, Lophophora williamsii
  source 1..1095: mol_type = mRNA
atgggctcagcctcaggctcagctgagagaacccagatgggtgaggacgaggcctgctcattcgccatgaccatcaccagtgggtccgtcccacccatggtgctcaaggccgtaattgagctcgatgttctggagatcattaagcgggccgggcctggtgcccacctctcccccgccgagatcgccgcccagctccccaccaccaacccgggtgccgccgccatgttggaccggatgctccgcctcctcgccagctacgacgtcctctcctactccctccacaccctccccgatggccgggtcgagaggctctatgggctcgcccctgtttgccagttcttgaccaacaatgaagatggtgtcaccttgagtgctctctctctcatgaaccaggacaaagttctcatggagagctggtaccacttgaaagacgcggttcttgatggtggtattccattcaacaaggcttatggcatgactgcatttgagtaccatgggactgatcctagatttaacaaggtcttcaacaatggaatgtccaaccattctaccattaccatgaagaaactccttgaaaattacaagggatttgagggtgtctccacattggttgatgtaggaggaggcactggagctaccctcaacatgattatctcaaagcaccctacaattaagggtattaacttcgatttgcctcatgtcattgaagatgcaccaacatatccaggagtggagcatattggaggagatatgttcgtgagtgtgccaaaaggagacgccatatttatgaagtggatttgtcacgactggagtgatgaacattgcctaaggtttttgaagaattgttatgccgccttgcccgaccatgggaaggttatcgtctgtgaatacatccttcctgtggctccagaaaccaatcatgctgcgaggacagtctttcatgttgatgcgatcatgttggctcataacccaggtgggaaagaaaggactgagcaagagtttgagtccctggctaaaggcgctggatttgaaggctttcgagtggcttgttcagcttatggtaccaaggtcatggaatttttgaaaaagaactaa

SEQ ID NO: 18 - RNA, 1095 nt, Lophophora williamsii
  source 1..1095: mol_type = mRNA
atggcttcagccaacaccaatggcagtgccaagggtagcagtaccgaggacagggaatggatctatgcgatgatcctgaccaccttctcggctccggccatggttctcaagactgtcctcgagctggatgtcctcgagatcatcaagaaggctgggccggacgcccaactctcatccgctgagattgtggcacagctccccactcagaaccctgaggctccttccatgatggaccgcatgctccgcctcctcgccagctactctgtcctccactgctcccttcggaccctccccgacggccgggtcgggaggctttacgggctcgctccggtgtgcaagtacttgacgaaggatgaggatggcctctccttggccagtcactctctttttatccaagacgaggtttccatggagtgctggtatcatgtaaaagatgcacttttggagggaggcagcccgttttacaagctgcatggcaagtcaccgtatgcatactacggaacggatggcaggtccaacgtgctcttcaacaacgcaatggcggacatgtccacccttgtcatgaagaagatcatcgagagctacaagggatttgagggcgtcaacactttggtcgacatgggcggcaacagaggggcgtcccttagcttgattgtctccagatatcctcacataaagggtatcaattttgacctgccccatgtcgtcagtgatagatcagactttccaggaattacccatgtcgccggagatatgtttgtgagcgtaccacaaggagatgctatctttgtcaagaatgtcttccacgattgggatgacgaacactgcttgacgatcatgaaaaattgctacgctgccctgccggatcatggcaaggtaatcgtctgtgagtttatccttcctgaagcaccggattctgaagatgctacaaaaatggcctaccagtttgatgtaataatgatgattggcccgaatggaaaagaaaggactcagccagaatttgaagctttgggcacagctgcgggatttgaaagttttagagtggcgtgttcggattatgacttcaagctcatggagttcatcaagaagaattag

SEQ ID NO: 19 - RNA, 1107 nt, Lophophora williamsii
  source 1..1107: mol_type = mRNA
atggctccatccattacaaatgagaaccccatcaacagcaatgtaaagggtagtaccgaggacagggaatggatctatgcgatgatcctgaccaccttctcggctccggccatggttctcaagactgtcctcgagctggatgtcctcgagatcatcaagaaggctgggccggacgcccagctctcatccgctgagattgtggcacagcttcccactcagaaccctgaggctccttccatgatggatcgcatgctccgcctcctcgccagctactctgttctcagctgctcccttcggaccctccccgactgccgggtcgagaggctttacggccttgctccggtctgcaagtacttgactaaggatgaggatggcctctccttggccagtcactctctttttatccaggacgaggtttccatggagtgctggtatcatgtaaaagatgcacttttggagggaggcagcccgttttacaagctgcatggcaagtcaccgtatgcatactacggaaccgatggcaggtccaacgtgctcttcaacaacgcaatggcggacatgtccacccttgtcatgaagaagatcatcgagagctacaagggatttgagggcgtcaacactttggtcgacatgggcggcaacagaggggcgtcccttagcttgattgtctccagatatcctcacattaagggtatcaattttgacctgccccatgtcgtcagtgatagatcagactttccaggaattatccatgtcgccggagatatgtttgtgagggtaccacaaggagatgctatctttgtcaagaatgtcttccacgattgggatgacgaacactgcttgacgatcatgaaaaattgctacgctgccctgccggatcatggcaaggtaatcgtctgtgaatttatccttcctgaagcaccggattctgaagatgctacaaaaatggcctaccaatttgatgtaataatgatgattggtccgaatgggaaagaaaggactaagccagaatttgaggctttgggcaaagctgcaggatttgaaggttttagagttgcgtgttcggattatgacttcaagctcatggagttcatcaagaagaattag

SEQ ID NO: 20 - RNA, 1131 nt, Lophophora williamsii
  source 1..1131: mol_type = mRNA
atggcaccaacatcaacaaactacgaagcagaaaacatctcctactgggatcttcaagaagaagatgatgaagctgcatgctcctttgctctgtctttgattggcgatgcattcaaatttgcagcgttaaaaacagccatggaacttgacatcattgagatgatcaagaagtctggccgcggtgctcgtcttactccggctgagatcgccgccaagctgcccaccaacaacccggcggcccccaccatgctccgccgcttgcttgactacttagtcagccactccatcttcaccgtcgctcctaccaccctccctgacggctctgtcgagaggcggtatggccttgcacctgtgtgtaagttcctgacccccaatgacgatggtgtctccttggctcccttcattagcaatcgtaacatgcacaagtgtctcgttccaagcaccttacatttgaaagatgcagttcttgaaggcggggagccattcgagaaggcttacggaatgagcctctatgagtacagtggcaagcatcccgagcatcagacagattttcacaaggccatgtctgatcactcaacccttgtcctgaagaagctcctcaagacctacaaagggcttgaaggtctgtcttctttggtcgatgttggtggtggcaatggagctaccctctccatgattctttctaagtaccccaagatcaaaggcatcaattttgatcagcctcatgtagttgcagatgcaccagtgtcccatccagggcttgaacatgttggaggagatatcttcgtcagtattcccaaaggagatgccatatttctcaagtggatcttgcattgcttcggagacgaagactgcataaaaatcctaaagaactgctatgctgcccttccagagggcggtggcaaggtcatagtctgtgagtacctgcttcctaatccggatgagactacccctgacatcgctgggaattcagtcgttcagttcgacatgtgcatgatggttgtgaccggggggatggagaggacggccaaggagttcgagacgttggcattagctgctgggtttcagggcttccaagttgttggctctgcttataatgtcaagatcatggagttactcaagaaaaactaa

SEQ ID NO: 21 - RNA, 1095 nt, Lophophora williamsii
  source 1..1095: mol_type = mRNA
atgggctcagcctcaggctcagctgagagaacccagatgggtgaggacgaggcctgctcattcgccatgaccatcaccagtgggtccgtcccacccatggtgctcaaggccgtaattgagctcgatgttctggagatcattaagcgggccgggcctggtgcccacctctcccccgccgagatcgccgcccagctccccaccaccaacccgggtgccgccgccatgttggaccggatgctccgcctcctcgccagctacgacgtcctctcctactccctccacaccctccccgatggccgggtcgagaggctctatgggctcgcccctgtttgccagttcttgaccaacaatgaagatggtgtcaccttgagtgctctctctctcatgaaccaggacaaagttctcatggagagctggtaccacttgaaagacgcggttcttgatggtggtattccattcaacaaggcttatggcatgactgcatttgagtaccatgggactgatcctagatttaacaaggtcttcaacaatggaatgtccaaccattcgaccattaccatgaagaaactccttgaaaattacaagggatttgagggtgtctccacattggttgatgtaggaggaggcactggagctaccctcaacatgattatctcaaagcaccctacaattaagggtattaacttcgatttgcctcatgtcattgaagatgcaccaacatatccaggagtggagcatattggaggagatatgttcgtgagtgtgccaaaaggagatgccatatttatgaagtggatttgtcacgactggagtgatgaacattgcctaaagtttttgaagaattgttatgcggccttggccgaccatgggaaggttatcgtctgtgaatacatccttcctgtggctccagaaactaatcatgctgcgaggacagtctttcatgtcgatgcgatcatgttggctcataacccaggtgggaaagaaaggactgagcaagagtttgagtccctggctaaaggggctggatttgaaggctttcgagtggcctgttcagcttatgatactaaggtcatggaatttttgaaaaacaactaa

SEQ ID NO: 22 - RNA, 1095 nt, Lophophora williamsii
  source 1..1095: mol_type = mRNA
atggcttcagccaacaccaatggcagtgctaagagtagcagtaccgaggacagggaatggatctatgcgatgatcctgaccaccttctcggcttcggccatggttctcaagactgtcctcgagctggatgtcctcgagatcatcaagaaggctgggccggacgcccagctctcgcccgccgagatcgtggcccagctccctactcagaaccctgaggctccttccatgatggaccgcatgctccgcctcctcgccagctactctgttctcagctgctcccttcggaccctcgccgatggccgcgtcgagaggctttacggcctcgctccggtctgcaagtacttgacgaaggatgaggatggcctctccttggctagccactctctttttatccaagacgaggtttccatggagtgctggtatcatgtaaaggatgcacttttggagggaggcagcccgttttacaagctgcatggcaagtcaccgtatgcatactacggaacggatggcaggtccaacgtgctcttcaacaacgcaatggcggacatgtccacccttgtcatgaagaagatcatcgagagctacaagggatttgagggcgtcaacactttggtcgacatgggcggcaacagaggggcgtcccttagcttgattgtctccagatatcctcacattaagggcatcaattttgacctgccccatgtcgtcagtgatagatcagactttccaggaattacccatgtcgctggagatatgtttgtgagcgtaccacaaggagatgctatctttgtcaagaatgtcttccacgattgggatgacgaacactgcttgacgatcatgaaaaattgctacgctgccctgccggatcatggcaagctaatcgtctgtgaatttatccttcctgaagcaccagattctgaagatgctacaaaaatggcataccagtttgatgtaataatgatgattggtccgaatggaaaagaaaggactaagccagaatttgaggctttgggcaaagctgcgggatttgaaagttttagagttgcgtgttcggattatgacttcaagctcatggagttcatcaagaagaattag

SEQ ID NO: 23 - RNA, 1104 nt, Lophophora williamsii
  source 1..1104: mol_type = mRNA
atggctccatccattacaaatgagaacactatcaacaacaatgtaaagggtagtaccgaggacagggaatggatctacgcgatgatcctgaccaccttctcggctccggccatggttctcaagactgtcctcgagctggatgtcctcgagatcatcaagaaggctgggccggactcccagctctcgcccgccgagatcgtggcccagctccccactcagaaccctgaggctccttccatgatggaccgcatgctccgcctcctcgccagctactctgttctcagctgctcccttcggaccctccccgacggccgggtcgagaggctttacgggctcgctccggtctgcaagtacttgacgaaggatgaggatggcctctccttggctagccactctctttttatccaagacgaggtttccatggagtgctggtatcatgtcaaagatgcacttttggagggaggcagcccgttttacaagctgcatggcaagtcaccgtatgcatactacggaactgatggcaggtccaacgtgctcttcaacaacgcaatggcggacatgtccacccttgtcatgaagaagatcatcgagagctacaagggatttgagggcgtcaacactttggtcgacatgggcggcaacagaggggcgtcccttagcttgattgtctccagatatcctcacataaagggtatcaattttgacctgccccatgtcgtcagtgatagatcagactttccaggaattacccatgtcgccggagatatgtttgtgagcgtaccacaaggagatgctatctttgtcaagaatgtcttccacgattgggatgacgaacactgcttgacgatcatgagaaattgctacgctgccctaccggatcatggcaaggtaatcgtctgtgaatttatccttcttgaagcaccggattgtgaagatgctacaagaatggcctaccagtttgatgtaataatgatgattggtccgaatgggaaagaaaggactaagccagaatttgaggctttgggcaaagctgcaggatttgaaagctttagaattgcatgttcggattatgacttcaagctcatggagttcatcaagaagaat

SEQ ID NO: 24 - RNA, 1095 nt, Lophophora williamsii
  source 1..1095: mol_type = mRNA
atggcttcagccaacaccaatggcagtgccaagagtagcagtaccgaggacagggaatggatctatgcgatgatcctgaccaccttctcggctccggccatggttctcaagactgtcctcgagctggatgtcctcgagatcatcaagaaggctgggccggacgcccagctctcgcccgccgagatcgtggcccagctccccactcagaaccctgaggctccttccatgatggaccgcatgctccgcctcctcgccagctactctgttctcagctgctcccttcggaccctccccgacggccgggtcgagaggctttacgggctcgctccggtctgcaagtacttgacgaaggatgaggatggcctctccttggccagccactctctttttatccaagacgaggtttccatggagtgctggtatcatgtaaaggatgcacttttggagggaggcagcccgttttacaaactgcatggcaagtcaccgtatgcatactacggaacggatggcaggtccaacgtgctcttcaacaacgcaatggcggacatgtccacccttgtcatgaagaagatcatcgagagctacaagggatttgagggcgtcaacactttggtcgacatgggcggcaacagaggggcgtcccttagcttgattgtctccaaatatcctcacataaagggtatcaattttgacctgccccatgtcgtcagtgatagatcagactttccaggaattacccatgtcgccggagatatgtttgtgagcgtaccacaaggagatgctatctttgtcaagaatgtcttccacgattgggatgacgaacactgcttgacgatcatgagaaattgctacgccgccctgccggatcatggcaaggtaatcgtctgtgaatttatccttcctgaagcaccggattctgaagatgctacaaaaatggcctaccagtttgatgtaataatgatgattggtccgaatggaaaagaaaggactaagccagaatttgaggctttgggcaaagctgcgggatttgaaacttttagagttgcgtgttcggattatgacctcaagctcatggagttcatcaagaagaattag

SEQ ID NO: 25 - RNA, 1131 nt, Lophophora williamsii
  source 1..1131: mol_type = mRNA
atggcaccaacatcaacaaattacgaagcagaaaagatctcctactgggatcttcaagaagaagatgatgaagctgcatgctcctttgctctgtctttgattggcgatgcattcaaatttgcagcgttaaaaacagccatggaacttgacatcattgagatgatcaagaagtctggccgcggtgctcgtcttactccggcggagatcgccgccaagctgcccaccaacaacccggcggcccccaccatgctccgccgcttgcttgactacttagtcagccactccatcttcaccgtcgctcctaccaccctccctgacggctctgtcgagaggcggtatggccttgcatctgtgtgtaagttcctgacccccaatgacgatggtgtctccttggcttccttcattagcaatcgtaacatgcacaagtgcctcgttccaagcaccttacatttgaaagatgcagttcttgaaggcggggagccattcgagaaggcttacggaatgagcctctatgagtacagtggcaagcatcccgagcatcagacagattttcacaaggccatgtctgatcactcaacccttgtcctgaagaagctcctcaagacctacaaagggcttgagggtctatcttctttggtcgatgttggtggtggcaatggagctacgctctccatgattctttctaagtaccccaagatcaaaggcatcaattttgatcagcctcatgtagttgcggatgcaccagtgtcccatccagggcttgaacatgttggaggagatatcttcgtcagtattcccaaaggagatgccatatttctcaagtggatcttgcattgcttcggagacgaagactgcataaaaatcctgaagaactgctatgctgcccttccagaaggcggtggcaaggtcatagtctgtgagtacctgcttcctaatccggatgagactacccctgacatcgctgggaattcagtcgttcagttcgatatgtgcatgatggttgtgaccggggggatggagaggacggccaaggagttcgagacgttggcattagctgctgggtttcagggcttccaagttgctggctctgcttataatgtcaagatcatggagttactcaagaaaaactaa

SEQ ID NO: 26 - RNA, 1161 nt, Lophophora williamsii
  source 1..1161: mol_type = mRNA
atggctgaaattcccacatcatccaatccttctgacgatcttgaaacccaaaaactcaacggaaatgaagaggactacgatcatcaccatgatgaagaccctgaaagtgatgacgaaaactatgaatacgcactgcagatagcagagatgctgccgtttccaatggtgatgaaaactgcgatcgagctcgatctcctcgggatcatagccacggccgggccagaccgccagctctcggcggcggagattgctgcggccctccctgcggccgggaatcccgacgctccggcgatgttggaccggatgctctatcttctggccacctattccgtggttacttgcaccgccgtggacggcggagccagcggcggcgttgtgaggaagtatgggctagcacctgttgccaagtactttgtttccaacgaagatggggtttcattgggtgctctcatttcgttgaatcagggtcaagcatttcttgccagctggagcaagttaaaggaggcagcgttggaaggaggaatcccattcaacaaagttcatggaatggatgtatttcattaccaagggacagagcccagattcaacaagatattcaacaaggcaatgtacggtcagtccacgtacatcatcaagaagattgtgaggaggtataagggttttgagaatatccaaaggctcgtggatgtaggcggaggccttggccacactcttcgagttataacctctaattatccctcaatcaagggcatcaatttcgacttgcctcatgtcattcagcatgcccctaccattcctggtgttgaacatgttggcggagatatgtttgaaagcattccaaatggagatgccatcttcatgaagtgcatcctccacgattggagcgatgagcattgcctgaaaacactgaagaactgctacaaggcgctcccgagaaagggaaaggtgatagtggtgcagatgaacatgatagaggagccacagacaacgccattagcgaaagccatcagccaattggacgtatgtctgatgactcaaagccctggaggaaaggaaaggactcggagggaattccagactcttgccgaggctgctggatttgccgaattcaaccctgtttgccatgtcgctggtttctgggtcatggaattcttgaaatag

SEQ ID NO: 27 - RNA, 1075 nt, Lophophora williamsii
  source 1..1075: mol_type = mRNA
ccatgatgaagaccctgaaagtgatgacgaaaactatgaatacgcactgcagatagcagagatgctgccgtttccaatggtgatgcaaactgcgatcgagctcgatctcctcgggatcatagccacggccgggccagaccgccagctctcggcggcggagattgctgcggccctccctgcggccgggaatcccgacgctccggcgatgttggaccggatgctctatcttctggccacctattccgtggttacttgcaccgccgtggacggcggagccagcggtggcgttgtgaggaagtatgggctagcacctgttgccaagtactttgtttccaacgaagatggggtttcattgggtgctctcatttcgttgaatcagggtcaagcatttcttgccagctggagcaagttaaaggaggcagtgttggaaggaggaatcccattcaacaaagttcatggaatggatgtatttcattaccaagggacagaccccagattcaacaagatattcaacaaggcaatgtacggtcagtccacgtacatcatcaagaagattgtgaggaggtataagggttttgagaatatccaaaggctcgtggatgtaggcggaggccttggccacactcttcgagttataacctctaattatccctcaatcaagggcatcaatttcgacttgcctcatgtcattcagcatgcccctaccattcctggtgttgaacatgttggcggagatatgtttgaaagcattccaaatggagatgccatcttcatgaagtgcatcctccacgattggagcgatgagcattgcctgaaaacactgaagaactgctacaaggcgctcccgagaaagggaaaggtgatagtggtgcagatgaacatgatagaggagccacagacaacgccattagcgaaagccatcagccaattggacgtatgtctgatgactcaaagccctggaggaaaggaaaggactcggagggaattccagactcttgccgaggctgctggatttgccgaattcaaccctgtttgccatgtcgctggtttctgggtcatggaattcttgaaatga

SEQ ID NO: 28 - protein, 522 aa, Lophophora williamsii
MGSLDIKQESSPLMTNPLDSEEFRRQGYMVIDFLAEYYKNIQKFPVRSQVEPGYLRKRLPESAPYEPESIERILKDVHDDIVPGLTHWQSPNYYAYFPSSGSTAGLLGETLAAGFNVVGFNWISSPASTELESIVMDWLAEMLNLPKSFTFSGDGGGVMMGTTCEAILTTITAARDRILDRIGREHINKLVVYGSDQTHCSFFKSAKIAGILPNNFRQVKTSRVNAFSMRPDALRAAIQADVDAGLVPFFLCTTVGTTSTAAVDPVALLCEVAKDYGMWVHIDAAYAGNACICPEFRHMINGVENADSFSFNTHKWFLTTLDCCCLWVKDPSSLVRCLSTNPEYLKNKATDTQQVVDYKDWQITLSRRFRSLKMWLVLRSYGVDYLRNFLRSHVDMAAYFEQLVRMDGRFEVVVPRSFALVCFRLSASAVIQRLYDSCHRHGHGQKFLEEETLNDVNAELLESVNSAGQIYMTHSLVDGVYMLRFAVGATLTQTHHVTYAWKQVQDHASAVLARRINSICNC

SEQ ID NO: 29 - protein, 430 aa, Lophophora williamsii
MLSAGINIVGFSWMSCPAATELEIIVLDWLAKLLNLPDTFLSTGNGGGVIQGTASEAVLVALLAARDKKLKKIGKDSVGKLVVYSSDQTHSSVQKACQIAGIHPENFRILKTDSSTEYALSPGVVLNAISKDVASGLIPLLLCATVGTTSSTAIDPLVDLGKIAKSNRIWLHVDAAYAGSACICPEYRHYINGVEEADSFNMNAHKWLLTNFDCSALWIKDRNALIQSLSTYPEYLRNKASEANVVVDYKDWQIPLGRRFRSLKLWMVLRLYGQEGLQSYIRNHIALAKQFEELVIQDSRFEMVTPRRFSLVCFRLLPRSTDENQADKLNRALLDAVNLTGAIFISHTVLSGMYVLRLAVGAPLTEERHVIAAWKVLQEKATALLEGNVAQEPNGHAQLSNGVIELDGALDNGVTTEQQGHVDDLPIKSK

SEQ ID NO: 30 - protein, 530 aa, Lophophora williamsii
MDGQMLKPMDAEQLREYGHQMVDFVADYYKTIESFPVLSQVQPGYLRELVPTNPPTHPESLQDVLDDVKAKILQGVTHWQSPGYFAYFPCNSSTAGFLGELLSAGINIVGFSWMSCPAATELEVIVLDWLAKLLNLPDSFLSIGNGGGVIQGTASEAVLVALLAARDKKLKKIGKDSMRKLVVYSSDQTHSSVQKACQIAGIHPENFRILKTDSSTEYALSPEVVLNAILKDVASGLVPLFLCATVGTTSSTAIDPLLALGKIAKSNRIWLHVDAAYAGSACICPEYRHYINGVEEADSFNMNAHKWLLANFDCSALWIKDRNALIQSLSTYPEYLRNKASEANVVVDYKDWQIPLGRRFRSLKLWMVLRLYGQEGLQSYIRNHIALAKQFEELVIQDSRFEMVTPRRFSLVCFRLLPRSTDENQADKLNRALLDAVNLAGTVFISHTVLSGKYVLRLAVGAPLTEERHVIAAWKVLQEKATALLEGNVAQEPNGHAQLSNGVVEVDGALDNGVTTEQHGHVDDLPIKSK

SEQ ID NO: 31 - protein, 501 aa, Lophophora williamsii
MALVLLLVVVCSFIFFLVILPRKQGKLNLPPGPKGLPLIGNLHQFDSLAPHLYFSKLAKIYGPILSLRFGCRSAVVIQSAALAKEVLKAQDHNFCTRPTLVAQQRLSYNGSDIIFAPYNECFRELRKLCVVHLFSSKKVQSSTPIRQEEVSRMIKKITALSSASKVVNLSNLLMALTSSIICRVAFGKRYEYEGVEGSRFHEVLNEVQAIAAAPFFTDYFPFIGSWLDKLTGLSCRLDKVFKKMDKFYDEIINDHLDPNRPKDDSHEDIIDVLLQLRKERCFSFELTLDHIKAVLMDIIVAGTDTSAAIVVWTMTELMKHPTMMMKVQEELRTVVQDKTFINESDLSKLTYLKATVKETLRLHNAGPLLVVREVIKKCSIQGYDILPKSLVFVSIWEIGRDPKFWTDPEIFMPERFLGSSIDFKGQDFELIPFGAGRRICPGMLLGVANVELALANLLYTFEWKLPVGMTEEDIGFDVIPGITMHKKNPLCLVAKKFSHVN

SEQ ID NO: 32 - protein, 523 aa, Lophophora williamsii
MHELLAASMDSIRGSPTLMGVLLFLGPILLLGLISRLRRKPYPPGPKGLPIIGNMMMMGQLTHRGLAKLAQRYGGLFHLKMGLVHMVVVSGPEEARQVLQVQDNIFSNRPASIAISYLTYDRADMAFAHYGPFWRQMRKLCVMRLFSRKRAESWQSVRDEVDHIVRAMATQAGSPVNVGELVFNLTKNVTYRAAFGSNSSEGQDEFIAILQEFSKLFGAFNIADFIPFLTWVDPNGLNDRLAKARGALDRFIDKIIDDHMEKKKANGHIKGGGVKEEEDMVDDLLNFYSEEAKVTESEDLQNAIKLTRDNIKAIIMDVMFGGTETVASAIEWAMAELMHCPEDMKRVQQELADVVGLTRRVQESDLDQLPFFRCCIKETLRLHPPIPLLLHETAEPAMIGGYQIPKRARVMINAWAIGRDPNSWSEAETFRPSRFLQDGVPDFKGGNFEFIPFGSGRRSCPGMALGLYALEMCVANLLHCFKWELPDGMKPSELDMSDTFGLTAPRASRLVAVPSPRLLCPLN

SEQ ID NO: 33 - protein, 533 aa, Lophophora williamsii
MQQLHSFIGSLLVSSKDDPTKMVSRLLFLFIAPVAALLLIVRRARRKPYPPGPTGLPLIGNMMMMDQLTHRGLARLAKRYGGLFHLKMGFVHMIVVSSPDEARQVLQAQDHIFSNRPATIAISYLTYDRADMAFAHYGPFWRQMRKLCVMRLFSRKRAESWQSVRDETDRIVRAMAARTGSPVNVGEHVFNLTKNVIYRAAFGSSSSEGQEEFIAILQEFSKLFGAFNIADFIPFLWWVDPNGLNSRLVKARAALDRFIDNIIDEHISYKQKKPNGVVKEEADMVDDLLNFYSGDEMGKVSESDDLQNAIKLTRDNIKAIIMDVMFGGTETVASAIEWAMAELIRSPDDLKRVQKELEDVVGLTRLVDESDLDKLTYFRCCIKETLRLHPPIPLLLHETAQEAVISGYRIPKQARVMINAFAIGRDPNSWSDPDSFKPGRFLNEGVPDFKGGNFEFIPFGSGRRSCPGMQLGLYALEMCVANLLHCFTWDLPDGMKPSELSMGDIFGLTAPRATRLIAVPTPRLLCPLYEHAP

SEQ ID NO: 34 - protein, 529 aa, Lophophora williamsii
MQQLHSFIGSLLVSSKDDPTKMFSRLLFLFIAPVAALLLIVRRARRKPYPPGPTGLPLIGNMMMMDQLTHRGLARLAKRYGGLFHLKMGFVHMIVVSSPDEARQVLQAQDHIFSNRPATIAISYLTYDRADMAFAHYGPFWRQMRKLCVMRLFSRKRAESWQSVRDETDRIVRAMATRTGSPVNVGEHVFNLTKNVIYRAAFGSSSSEGQEEFIAILQEFSKLFGAFNIADFIPFLWWVDPNGLNSRLVKARAALDRFIDNILDEHISYKKKRPNGVVKEEADMVDDLLNFYSGDEMGKVSESDDLQNAIKLTRDNIKAIIMDVMFGGTETVASAIEWAMAELIRSPDDLKRVQKELEDVVGLTRLVDESDLDKLTYFRCCIKETLRLHPPIPLLLHETAQEAVISGYRIPKQARVMINAFAIGRDPNSWSDPDSFKPGRFLNEGVPDFKGGNFEFIPFGSGRRSCPGMQLGLYALEMCVANLLHCFTWDLPDGMKPSELSMGDIFGLTAPRATRLIAVPTPRLLCPLY

SEQ ID NO: 35 - protein, 500 aa, Lophophora williamsii
MDMEIACCFVAVLASIFFLCKLLFAAKSKQRNKKSLPPSPPTIPILGHLHLLKPPFHRTLHALAQRYGPIFYLRLGSQPAVVVSAPWTAEECFTRNDVVFADRPKFLVGHHLGYNNSILLWSPYGPHWRNLRRLATITALSQKQINLMGPARRVEVRNMIRDLFRSSESGTRNVNLNEAFAKLTRNLVMRAVNGRPWETTIMTPPSQLMTVCDFVPVLRWAGYKGIEKEMIKLRKERDGVLQSLLDEYRESRAIRRTGGVDGDDAEKKTMMDDLLDLQEAEPEYYTDDTLKGLVLVMLLAGSDTSARTLEWAMALLLNHPEVLEKAKTEIDTHIDKGRIVEDSDLPRLTYISCIINETLRLFPPAPLLVPHYSSQDCTIGGYHVPKGTLLLVNAWAIHRDPALWDEPTKFKPERFEKDKLEGFSFKYFPFGMGRRACPGTNFALRNVTLVLATLLQCFDWKVAEPVDLNERAGAIIMPKEKPLHAICHPRPSMMGTLSQI

SEQ ID NO: 36 - protein, 508 aa, Lophophora williamsii
METWFLIIVTACLGALLKAFLSLKPKPKLPPGPGGVPIFNSIKWLGASSTDIESYLRNLRQKFGPIITIHMNTRPAIFVSSHTIAHQALVQNGAIFSDRPKPTATGKIATANQHNISSAGYGPTWRTLRRNLTSQILHPSRLKEYSHARKWVLDILITRFGSDGDDAVKVVDHFQFAMFCLLVLMCFGDKLEESKIKEVEDVQRSLLVSFTKFQLLNFFPRLSRLLFWKRWELFFRLRKRQQEVLIPLIRARKEINKQDGEKPVTSYVDTLLNLEIQDDDSGSKRKLTEDELISACSEFLSAGTDTTSTALQWIMANLVKYPEIQTRLFAEIRGVIGAEAEHVGEEDLPKMPYLKAVVLEGLRRHPPGHFVLPHTVIQEADLGGYVVPKNAVINFMVAEMGWDPQVWEDPMEFKPERFLPANGSDVEFDITGSREIKMMPFGAGRRICPGFGLAILHLEYFVANLVWKFEWAAAKGHEVDLSEKQEFTIVMKHPLHARISPRRLSSSN

SEQ ID NO: 37 - protein, 437 aa, Lophophora williamsii
MEYYNCITLFVALLIITLHFLIRNPRKSKLPPGPKPWPIIGNIHLLGSKPHRSVADLSKTYGPLMTLRLGSITTIVISSPDVAKEMFLKHDVDFASRQIPDCVWALDHAKFSMAWLPVCPKWRELRKISSIQLFTNQRLDASQGLRKEKVNGLVEFVKGCCEKGLAVDIGKAAFTTALNLLSNTFFSMDLCAYDSSGSQEFKDLAWHIMEEIGRPNISDFFPLIKALDLQGVKRSISTSFSKMLEVFDKIIHERLSDQIGSKDDVLDTLLNLVKQNELTLDDVRHMLIDLFVAGTDTTSNTIEWALTELLRHPEKMTKAQAEIEQLLEKGQSRSIQESDISKLPYIQAIVKETMRLHPAAPFLIPHKATKNIQLCGYCVPKNAQIWVNSWSMGRDPRVWPDSESFSPERFLESEIDIKGRDFTLLPLSWYPIRPRRI

SEQ ID NO: 38 - protein, 517 aa, Lophophora williamsii
MDYPALFFYIFTFLLSYILTQTLLRKFQNLPPSPFPTLPILGHLYLLRNPSSAHRTLANLSAHYGPILFLQLGARPALLVSSPSAAEECLGRNDVVFANRPRLVGAKILGYEYTNILWAPYGPLWRTHRRIAANEILSPHRLQLLSWIRADEVRSLIRRIMGLSGDQDGTVEIKPALFELTNNVMMRMIAGKRFFGEGSETSEVAKRFKEMLRELMGLGGAMTGADLFPYLRPLGLFKGTERKIKEVFGKMDRIFQDLVDEKRTKIMEEGTDENGGGNEEKSKTLIEVLLQLQKSDPTYSSDTIVKGLVQILLLAGSETSASTIEWALSLLVNNPQVLIKAQREIDEHIGFDRLLEENDLNHLRYLHCIINETLRMYPAAPLLPPHESSADCVVGGYRIPRATMLMVNVWAIQNDPKIWDEPSVFKPERFWDLEGEHKLGCKFMPFGSGRRACPGENLAMRVVGLALGSLIQCFDWKRVGEEMVDMEEKLGVGMWKAKPLQVKCRPRQQIYNLISSI

SEQ ID NO: 39 - protein, 504 aa, Lophophora williamsii
MDLVLLQKILYALFFAITVAIVVSKLKGKKLKLPPGPLPVPVFGNWLQVGDDLNHRNLTELAKRFGDILLLRMGQRNLVVVSSPELAKEVLHTQGVEFGSRTRNVVFDIFTGKGQDMVFTVYGEHWRKMRRIMTVPFFTNKVVQQYRSGWEAEAAAVVADVKKNPESATTGIVLRRRLQLMMYNNMYRIMFDRRFESEDDPLFNKLRALNGERSRLAQSFEYNYGDFIPILRPFLRGYLKICREVKERRLQLFKDYFVDERKKLASTKPTSNNELKCAIDHILDAQQKGEINEDNVLYIVENINVAAIETTLWSIEWAIAELVNHPHIQNKLREELDTVLGKGHQITEPDTHKLPYLQAVIKETLRLRMAIPLLVPHMNLHDAKLNGYDIPAESKILVNAWWLANNPAHWKNPEEFRPERFFEEESKVEANGNDFRYLPFGVGRRSCPGIILALPILGITIGRLVQNFEMLPPPGQSKLDTTEKGGQFSLHILKHSTIVLKPRD

SEQ ID NO: 40 - protein, 509 aa, Lophophora williamsii
MEELQLFSCAIILLVIVIAHKLFKKPKHQERNLPPSPPSLPIIGHLHMLKEPLHKTIHDISTKYGEVLLLKLGVRRALIIASPSAVEECLLKNDTTFANRPDTLVGRLLHYNSTSIAFSPYNGHYRTLRRVMTQEVFSSTKVPLFSGIRQAEVRLLLRDIWEECTTTGDVDTKTIHLNRGFNELALNIMTNMVAGKRYYGKDVEDVTEAKRFSEIIREGAVLHGAANPGDLFPMLKWMFRGLEKRMVTWMKSTDAFYQKLLDERKQTMSKDNTRAIVDVLIKLQQQDPAFFTNEVIKGMIMVLILAGADTSSATMEWSMALLLNNPHCLKKVQEEIDTVIGHNRLINEEDVAKLPYLQNIVMETLRLYPPTPLLLPHETSEDCNICGYDIPQGTMLMVSLWTLHRDPRWWNEPEKFIPERFEGKEGESLYKLLPFGLGRRNCPGYAMGKRAVALVLGSVLQCFDWRRPSDEMLNMSQSSGITMPKAQPLEAFCTIRPCMADLLSKELTC

SEQ ID NO: 41 - protein, 500 aa, Lophophora williamsii
MDYLSFLLCLAMALVAMHFLLSMARKGKESNPRRLPPGPSPLPIVGNLFSLGSKPHVSLTELAKTYGPLMILQLGWVPTVIISSAAVAKEALQKNDLSFSNRTVLDAACALDHHQNSIAFLPAASRWRNFRKICNSHVFSTSRLDASQCLRRNKVKDLLSYVEKCSKDGVVVDVGQLAFVTTLNLLSNTFFSVDLADFSSESAHQFKEIVRSVLEEVGKPNVADYFPLLRRMDPQGIRQRVGVHFQKMIDTLNSMINRRLEGTRPSTSLQAHDVLDALLDISHDKTEDIEQSHIPHLLLDLFVAGTDTTSSTLEWAMAELLRSPDKMKLAQAELEEIIGKGNPVEEPDIVRLPYLQAVVKETLRLHPAVPFLVPRKADSDVSLSGFTVPKNAQVLVNVWAIGKDPESWHNPSSFQPERFLGSECDVKGRDFELIPFGAGRRICPGLPLAIRMIPLMLGSLIHEFNWEGEGQTPPEMMDMEERFGFTLVRAQHLRAIPLRL

SEQ ID NO: 42 - protein, 501 aa, Lophophora williamsii
MGYYAIFAVVLPFLWTCFYLLKLNPTSKLTSTIRLPPGPRPLPIIGNLHQLGERPHRTLANLAKVYGPIISLKFGSITTIAVSSADVAKEMFQKHDLALASRKVPAAVRANGHDNFSVAWLPVTPKWRFLRKISAIQLFSTQRLDARQSLRQAKVVELLDYIKARSNAGEPVDIGEAAFTTSLNLLSNTFFSMDLASYSSAASGEFKELVWKIMEEIGKPNLADCFPLVGFISRMSVNRELMGYGNKLNEVFAEIIEKRLSAANSSEGRGNGDVLDTLLRIMEEDDSELSLDDIMHLLMDFFTAGTDTTSSTLEWAMTELLHNPEKMAKAQAELEQVLGKDTVSIQESDIAKLLYLQATVKETLRMHPPTVFLLPRKADSDIDLYGHLVPKNAQVFVNLWAISYDPSTWENPDLFSPERFLDQDIDMKGQDFGFIPFGAGRRICPGLTLAYRMLNLMLGTLIHVFNWKLGDGLSREDLDMTDKFGITIQKAKPLRAIPILK

SEQ ID NO: 43 - protein, 355 aa, Lophophora williamsii
MGRLLQMPYEMSVKIMVGSLEKNWLPDAVIRRLTRLLLASRLRSGYKLTSQLQLSDLLHFVHSLREMPIAIQTDKPKSQHYELPTSFFKLVLGKHMKYSCCYFSDEVTNLDAAEKAMLNLYCERAQLKDGHAVLDVGCGWGSLVLYVAQNYSNCRVTGICNSVTQKAFIEERCRDLELQNVEIKLADISTFEMEGSYDRVFSIEMFEHMKNYQDLLKKISNWMKPDGLLFIHYFCHKAFAYHFEDVNDDDWITRYFFTGGTMPSANLLLYFQDDVSVVNHWLVDGKHYAQTSEEWLKRMDSNLGSIRPIMESTYGKDCATKWTVYWRTFFIAVAELFGYNNGDEWMVAHFLFKKK

SEQ ID NO: 44 - protein, 364 aa, Lophophora williamsii
MGSASGSAERTQMGEDEACSFAMTITSGSVPPMVLKAVIELDVLEIIKRAGPGAHLSPAEIAAQLPTTNPGAAAMLDRMLRLLASYDVLSYSLHTLPDGRVERLYGLAPVCQFLTNNEDGVTLSALSLMNQDKVLMESWYHLKDAVLDGGIPFNKAYGMTAFEYHGTDPRFNKVFNNGMSNHSTITMKKLLENYKGFEGVSTLVDVGGGTGATLNMIISKHPTIKGINFDLPHVIEDAPTYPGVEHIGGDMFVSVPKGDAIFMKWICHDWSDEHCLRFLKNCYAALPDHGKVIVCEYILPVAPETNHAARTVFHVDAIMLAHNPGGKERTEQEFESLAKGAGFEGFRVACSAYGTKVMEFLKKN

SEQ ID NO: 45 - protein, 364 aa, Lophophora williamsii
MASANTNGSAKGSSTEDREWIYAMILTTFSAPAMVLKTVLELDVLEIIKKAGPDAQLSSAEIVAQLPTQNPEAPSMMDRMLRLLASYSVLHCSLRTLPDGRVGRLYGLAPVCKYLTKDEDGLSLASHSLFIQDEVSMECWYHVKDALLEGGSPFYKLHGKSPYAYYGTDGRSNVLFNNAMADMSTLVMKKIIESYKGFEGVNTLVDMGGNRGASLSLIVSRYPHIKGINFDLPHVVSDRSDFPGITHVAGDMFVSVPQGDAIFVKNVFHDWDDEHCLTIMKNCYAALPDHGKVIVCEFILPEAPDSEDATKMAYQFDVIMMIGPNGKERTQPEFEALGTAAGFESFRVACSDYDFKLMEFIKKN

SEQ ID NO: 46 - protein, 368 aa, Lophophora williamsii
MAPSITNENPINSNVKGSTEDREWIYAMILTTFSAPAMVLKTVLELDVLEIIKKAGPDAQLSSAEIVAQLPTQNPEAPSMMDRMLRLLASYSVLSCSLRTLPDCRVERLYGLAPVCKYLTKDEDGLSLASHSLFIQDEVSMECWYHVKDALLEGGSPFYKLHGKSPYAYYGTDGRSNVLFNNAMADMSTLVMKKIIESYKGFEGVNTLVDMGGNRGASLSLIVSRYPHIKGINFDLPHVVSDRSDFPGIIHVAGDMFVRVPQGDAIFVKNVFHDWDDEHCLTIMKNCYAALPDHGKVIVCEFILPEAPDSEDATKMAYQFDVIMMIGPNGKERTKPEFEALGKAAGFEGFRVACSDYDFKLMEFIKKN

SEQ ID NO: 47 - protein, 376 aa, Lophophora williamsii
MAPTSTNYEAENISYWDLQEEDDEAACSFALSLIGDAFKFAALKTAMELDIIEMIKKSGRGARLTPAEIAAKLPTNNPAAPTMLRRLLDYLVSHSIFTVAPTTLPDGSVERRYGLAPVCKFLTPNDDGVSLAPFISNRNMHKCLVPSTLHLKDAVLEGGEPFEKAYGMSLYEYSGKHPEHQTDFHKAMSDHSTLVLKKLLKTYKGLEGLSSLVDVGGGNGATLSMILSKYPKIKGINFDQPHVVADAPVSHPGLEHVGGDIFVSIPKGDAIFLKWILHCFGDEDCIKILKNCYAALPEGGGKVIVCEYLLPNPDETTPDIAGNSVVQFDMCMMVVTGGMERTAKEFETLALAAGFQGFQVVGSAYNVKIMELLKKN

SEQ ID NO: 48 - protein, 364 aa, Lophophora williamsii
MGSASGSAERTQMGEDEACSFAMTITSGSVPPMVLKAVIELDVLEIIKRAGPGAHLSPAEIAAQLPTTNPGAAAMLDRMLRLLASYDVLSYSLHTLPDGRVERLYGLAPVCQFLTNNEDGVTLSALSLMNQDKVLMESWYHLKDAVLDGGIPFNKAYGMTAFEYHGTDPRFNKVFNNGMSNHSTITMKKLLENYKGFEGVSTLVDVGGGTGATLNMIISKHPTIKGINFDLPHVIEDAPTYPGVEHIGGDMFVSVPKGDAIFMKWICHDWSDEHCLKFLKNCYAALADHGKVIVCEYILPVAPETNHAARTVFHVDAIMLAHNPGGKERTEQEFESLAKGAGFEGFRVACSAYDTKVMEFLKNN

SEQ ID NO: 49 - protein, 364 aa, Lophophora williamsii
MASANTNGSAKSSSTEDREWIYAMILTTFSASAMVLKTVLELDVLEIIKKAGPDAQLSPAEIVAQLPTQNPEAPSMMDRMLRLLASYSVLSCSLRTLADGRVERLYGLAPVCKYLTKDEDGLSLASHSLFIQDEVSMECWYHVKDALLEGGSPFYKLHGKSPYAYYGTDGRSNVLFNNAMADMSTLVMKKIIESYKGFEGVNTLVDMGGNRGASLSLIVSRYPHIKGINFDLPHVVSDRSDFPGITHVAGDMFVSVPQGDAIFVKNVFHDWDDEHCLTIMKNCYAALPDHGKLIVCEFILPEAPDSEDATKMAYQFDVIMMIGPNGKERTKPEFEALGKAAGFESFRVACSDYDFKLMEFIKKN

SEQ ID NO: 50 - protein, 368 aa, Lophophora williamsii
MAPSITNENTINNNVKGSTEDREWIYAMILTTFSAPAMVLKTVLELDVLEIIKKAGPDSQLSPAEIVAQLPTQNPEAPSMMDRMLRLLASYSVLSCSLRTLPDGRVERLYGLAPVCKYLTKDEDGLSLASHSLFIQDEVSMECWYHVKDALLEGGSPFYKLHGKSPYAYYGTDGRSNVLFNNAMADMSTLVMKKIIESYKGFEGVNTLVDMGGNRGASLSLIVSRYPHIKGINFDLPHVVSDRSDFPGITHVAGDMFVSVPQGDAIFVKNVFHDWDDEHCLTIMRNCYAALPDHGKVIVCEFILLEAPDCEDATRMAYQFDVIMMIGPNGKERTKPEFEALGKAAGFESFRIACSDYDFKLMEFIKKN

SEQ ID NO: 51 - protein, 364 aa, Lophophora williamsii
MASANTNGSAKSSSTEDREWIYAMILTTFSAPAMVLKTVLELDVLEIIKKAGPDAQLSPAEIVAQLPTQNPEAPSMMDRMLRLLASYSVLSCSLRTLPDGRVERLYGLAPVCKYLTKDEDGLSLASHSLFIQDEVSMECWYHVKDALLEGGSPFYKLHGKSPYAYYGTDGRSNVLFNNAMADMSTLVMKKIIESYKGFEGVNTLVDMGGNRGASLSLIVSKYPHIKGINFDLPHVVSDRSDFPGITHVAGDMFVSVPQGDAIFVKNVFHDWDDEHCLTIMRNCYAALPDHGKVIVCEFILPEAPDSEDATKMAYQFDVIMMIGPNGKERTKPEFEALGKAAGFETFRVACSDYDLKLMEFIKKN

SEQ ID NO: 52 - protein, 376 aa, Lophophora williamsii
MAPTSTNYEAEKISYWDLQEEDDEAACSFALSLIGDAFKFAALKTAMELDIIEMIKKSGRGARLTPAEIAAKLPTNNPAAPTMLRRLLDYLVSHSIFTVAPTTLPDGSVERRYGLASVCKFLTPNDDGVSLASFISNRNMHKCLVPSTLHLKDAVLEGGEPFEKAYGMSLYEYSGKHPEHQTDFHKAMSDHSTLVLKKLLKTYKGLEGLSSLVDVGGGNGATLSMILSKYPKIKGINFDQPHVVADAPVSHPGLEHVGGDIFVSIPKGDAIFLKWILHCFGDEDCIKILKNCYAALPEGGGKVIVCEYLLPNPDETTPDIAGNSVVQFDMCMMVVTGGMERTAKEFETLALAAGFQGFQVAGSAYNVKIMELLKKN

SEQ ID NO: 53 - protein, 386 aa, Lophophora williamsii
MAEIPTSSNPSDDLETQKLNGNEEDYDHHHDEDPESDDENYEYALQIAEMLPFPMVMKTAIELDLLGIIATAGPDRQLSAAEIAAALPAAGNPDAPAMLDRMLYLLATYSVVTCTAVDGGASGGVVRKYGLAPVAKYFVSNEDGVSLGALISLNQGQAFLASWSKLKEAALEGGIPFNKVHGMDVFHYQGTEPRFNKIFNKAMYGQSTYIIKKIVRRYKGFENIQRLVDVGGGLGHTLRVITSNYPSIKGINFDLPHVIQHAPTIPGVEHVGGDMFESIPNGDAIFMKCILHDWSDEHCLKTLKNCYKALPRKGKVIVVQMNMIEEPQTTPLAKAISQLDVCLMTQSPGGKERTRREFQTLAEAAGFAEFNPVCHVAGFWVMEFLK

SEQ ID NO: 54 - protein, 386 aa, Lophophora williamsii
MAEIPTSSNPSDDPETQKLNGNEEDYDHHHDEDPESDDENYEYALQIAEMLPFPMVMQTAIELDLLGIIATAGPDRQLSAAEIAAALPAAGNPDAPAMLDRMLYLLATYSVVTCTAVDGGASGGVVRKYGLAPVAKYFVSNEDGVSLGALISLNQGQAFLASWSKLKEAVLEGGIPFNKVHGMDVFHYQGTDPRFNKIFNKAMYGQSTYIIKKIVRRYKGFENIQRLVDVGGGLGHTLRVITSNYPSIKGINFDLPHVIQHAPTIPGVEHVGGDMFESIPNGDAIFMKCILHDWSDEHCLKTLKNCYKALPRKGKVIVVQMNMIEEPQTTPLAKAISQLDVCLMTQSPGGKERTRREFQTLAEAAGFAEFNPVCHVAGFWVMEFLK

SEQ ID NO: 55 - RNA, 1104 nt, Lophophora williamsii
  source 1..1104: mol_type = mRNA
atgggttcgtaccatgatgagaaaaatgtgatgggtggttcggatcagggggagctctgctccctagccatgaccatagctagcgggtcggtcccggctatggtgcttaaggccttgattgagctcgatgtgcttgagctcatcaagcgagctgggccaggggcccaactctccccggcccagatcgtggcccagctgcccactaagaacccggatgccggtaccatgttggaccggatgcttcggcttctcgccagctatgagatcttgtcctgttcgacccagaccctccctgatgggcgggtcgagaggctgtacgggcttgccccggtttgtcagttctttactaacaatgaagatggggcaaccttgggtgccatagttcagctaagccaggagaagttggttgtagagagctggtatcacttaaaagatgccgttcttgatggtggcattccattcaaaaaggctcgtggcatgactatatatgagtacccaaagacagatcctagattcaaccaagtcttcaatagtgggatgtccaactattcaaccattatcatgagggatctgctcgaacgctacaagggatttgagggtatttccaccttggttgacatagccggtggcaccggagcaagcattaacatggttgtttcaaaatatcccaccattaagggtatcaactttgatttgcctcacgtcatcaaagatgcaccttcctatgacggagtggagcatgttggaggagatatgtttgagagcgtgacaaaagcagacgcaatgctcataaagtggacgttgtgtgattggagcgatgagcattgcctcaagttgttgaagaactgctacaatgccctaccagacaatggtaaggtgatcgtctgtgattacgtccttcctatggctccggaaactagcaatgctgcaaagactgtcttcctcctggacgcgatcatgttagccaattttgtgggtggcaagggaagaacaaagcaagaatttgaagctttagccagaaaagtcggatttgagggctttgaagtgccttgttcatcatatgattttagcgtcatggaacttttcaagaaaaaaaataattaa

SEQ ID NO: 56 - RNA, 1509 nt, Lophophora williamsii
  source 1..1509: mol_type = mRNA
atggattactgcacaactttggcagccattcttctcttcctatggacttgtttctacttcatgaaacttaacccatttttctcaaaagccacctcgaccattcccctcccacctggtcctagacctctcccaatcatcggcaacctccaccagctcggtgagaggccttactgcaccttggctgacctatccagggcttatggccccataatatcgatcaagttcggaagcatcaccaccatagtgatttcatcttctgatgtagccaaagaaatgttccaaaaacacgaccttgccctgtctagcaggaaggtccctgcagcagtcagggcaaatgggcacgacaagttctcgattgcctggctgcctgtgacccccaaatggcggtatctcaggaagatctcaaccgttcaattgttctccacccaacgacttgacgctaggcagtcactccgccaggctaaagtagctgagctccttgactatgttagggtgtgttccaatgtaggagaatcagttgacattggagaagttgcctttacaactttacttaatttgttatcgaacgcatttttttctttggatttggctagttatagttcggtcgcatcaggtgagtttaaagggcttgtttggaagatagttgaggagattgggaagccgaatttggccgattttttccctcttctggggttcataagcaagctgagtgtgaatagggagttgatggggtatggaaataagttgaatgaggtgtttgcaaagatcatagaagaaagattaagtgcagctaattcatctgaaggtcgtgatgatggtgatgttttggataccctacttggactcatgaaagaagatgactctgagttgagtcttgatgatataaagcatctcctaatggacttctttacagctgggacagacacaacctcgagtactttagaatggacaatgacagagttactacacaatcccgaaaaaatgtcagaagctgaagctgagctcgaacgagtcctaggcagggaaactctccccattcaagaatctgacatcccaaaattgccttacttgcaggctgttgtgaaagagaccctaagaatgcacccacccaccgtatttctcctgcctcgcaaggcagattcagatgtagagttgtgtggctatttggtgcctaagaatgcccaagtgtttgtcaatctctgggccattagttatgacccaaatacctgggagaatccggatgtattttcacccgagaggttcttggatcaagatgtcgatatgaaaggccaagatttcgggttcataccatttggggcaggaaggagaatgtgccccggattgacattggcatataggatgttgaaccttatgttggggaccctgattcatgtcttcaattggaagcttgaggatggattgagtcatgagaatatggacatgagtgataagtttgggattactatacagaaggctaagcctctccgaattattccaatcccaaagtaa

SEQ ID NO: 57 - RNA, 1506 nt, Lophophora williamsii
  source 1..1506: mol_type = mRNA
atggactactacgcaattttggcagttgtacttcccttcctgtggacttgtttctacttgctgaaactcaacccaacctcaaaactcacgtcaaccacccgcctgccacctgggcctagacctctcccgatcatcggaaacctccaccaactcggcgagaggcctcaccgcaccttggctaacctagccaaggtgtacggccccataatatccctaaagttcgggagcataaccaccattgtggtatcctctgcggatgtagccaaagaaatgtttcaaaaacacgaccttgccctggccagcaggaaggtccctgcagcggtcagggcaaatgggcacgacaatttctccgttgcctggctgcctgtgacccccaaatggcggtttctcaggaagatctccgccatccaattgttctccactcaacgactcgatgctaggcagtcactccgccaggctaaggtagttgagctccttgactacgtcaaggcgcgttctagtgcaggagaaccagttgacattggagaggccgtgttcacaacttcacttaacttgttatcaaacacattcttttcaatgaatttggctagctatggttcagctgcgtcaggggagtttaaagagcttgtttggaagatgatggaggagatcgggaagccgaatttggccgattgttttcctcttgtagggttcataagcaggatgagtgtgaaccgggagttgatggtgtatggaaataagttgaatgaggtgtttgcagaaatcatagagaaaagactaagtgcagctaattcatctaaagatcgtggcaatggtgatgttcttgacaccctacttagaatcatggaagaagatgattctgagctcagtctggatgatatcatgcatcttctgatggacttctttacagccgggacagacacgacctcgagcaccttagaatgggcaatgacggagctactacataatcccgaaaaaatggcaaaagctcaagctgaggtcgaacaagtcctcggcaaggacactgtgttgattcaagaatctgacatcgcaaaactgccttacttgcaacctacagtgaaagagaccctaagaatgcacccacctaccgtatttctgttgcctcgaaaggcagactccggcgtagaactgtatggtcatttggtacccaagaatgcccaagtctttgtcaatctttgggcaattagctacgacccgagtacttgggagaacccggatttgttctcacccgagaggttcttggatcaagatatcgatatgaagggccaagatttcgggttcataccatttggagcaggaaggagaatatgtcctgggctgacattggcatgtaggatgttaaaccttatgctggcaactctaattcatgtcttcaattggaagcttggagatgggttgagtccggaagatttggacatgactgacaaatttgggattaccattcaaaaggctaagcctctccgggctattccaattctgaagtga

SEQ ID NO: 58 - RNA, 1506 nt, Lophophora williamsii
  source 1..1506: mol_type = mRNA
atggactactttccaattttggcatttgtacttcccttcctatggacttgtttctacttcctaaaactcaaccccacctcaaaactcacctcaaccatccccctcccaccgggtcctagacctctcccgatcatcggaaacctccaccagctcggcgagaggcctcaccgcaccctggctaacctagccaaggtgtacggccccataatatccctaaagttcgggagcataaccaccattgtggtatcctccgcggatgtagccaaagaaatgttccaaaaacacgaccttgccctggccagcaggaaggtccctgcggctgtcagggcaaatgggcacgacaatttctcgattgcctggctgcctgtgacccccaaatggcggtttctcaggaagatctccgccatccaattgttctccactccaagactcgacgctaggcagtcactccgccaggctaaggtagttgagctccttgactacatcaaggcccgctctaatgcaggagaaccggttgatattggagaggccgcgttcacaacttcacttaacttgttgtcgaacacatttttttcaatggacttggctagttatagttcagccgcatcaggagagtttaaagagcttgtttggaagataatggaggagattggaaagccgaatttggcggattgttttcctcttgtagggttcataagcaagctgagtgtgaaccgggagttgatggggtatggaaataagctgaatgaggtgtttgcagagatcatagagaagagactaagtgcagctaattcatctgaaggtcgtggtaatggtgatgttcttgataccctacttagaatcatggaagaagatgactctgagctcagtcgtgatgatatcatgcatcttcttatggacttctttacagccgggacagacacaacctcgagcaccttagaatgggcaatgacagagctgctacataatcccgaaaaaatggcaaaagctcaagctgagctcaaacatgtccttggcaaggacactgtgttgatcgaagaatctgacatctcaaaattgccttacttgcaagctacggtgaaagagaccctaagaatgcacccacctactgtatttctgttacctcgaaaggcagattctgatgtagaactgtatggtcatttggtgcctaagaatgcccaagtctttgtaaatctttgggcaattagctatgatccgagtacttgggagaacccgaatttgttctcacccgagaggttcttggatcaagatgtcgacatgaagggccaagatttcgggttcataccatttggagcaggaaggagaatatgtcctggaatgacattggcatataggatgttgaacctaatgttgggaactctaattcatgtgttcaattggaagcttggagatgggttgagtcctgaagatttggacatgactgacaaatttgggattaccgtacagaaggctaagccactccgcgctattccaattctaaagtaa

SEQ ID NO: 59 - RNA, 1518 nt, Lophophora williamsii
  source 1..1518: mol_type = mRNA
atggattactacacaactttggcagccattcttctgttcttatggacttgtttctacttcatgaaacgtaacccattttcctcaacagccaccacctcaaccatcctcctcccacctggtccgaaacctctccccatcatcggcaacctccaccagctcggtgggaggccttaccgcaccttggctgacctagccagggcttatggccccataatatcaatcaagttcggaagcgtcaccaccatagtggtttcatcttctgatgtagccaaagaaatgtttcaaaaacacgaccttgccctgtctagcaggaaggtccctgcagcggtcagggcaaatgggcacgacaacttctcgattgcctggttgcctgtgacccccaaatggcggtatctgaggaagatctcaaccgttcaattgttctccactcaacgacttgatgcgaggcagtcgctccgccaggctaaagtagctgagctccttgactacgttaggatgtgttccaatgtaggagaatcagttgacattggagaagttgcctttacaactcaacttaatttgttatcgaacacatttttttctttggatttggctagttacagttcagtcgcatcaggggagtttaaagagcttgtttggaagatagttcaggagattgggaagccgaacttggccgattttttcccttgtctggggttcataagcaagctgagtgtgaatagggagttgatggggtatggaaataagttgaatgagggatttgcaaagatcatagaagaaacgctaagtttcgctacttcatctgaaggtcgtggtgatggtgggggtgatgttctggataccctgcttagactcatgaaagaagatgactctgagttgagtcttgatgatataaagcatctcctcatggacttcttcacagctgggacagacacaacctcgagtaccttagaatgggcaatggcagagttactacacaatcccgaaaaaatgtcagaagctcaagctgagctcgaacgagtcttaggcagggatactctctccattcaagaatctgacatcccaaaattgccttacttgcaggctgttctgaaagagaccctaagaatgcacccacccaccgtgtttctcctgccccgcaaagcagattctgatgtcgagttgtgtggctatttggtgcccaagaatgcccaagtgtttgtcaatctctgggccattagttacgatccaaatacctgggagaatccggatgtattttcacccaagaggtttctggatcaagatgtcgatatgaaaggccaagatttcgggttcataccatttggggcaggaaggagaatgtgccccggattgacattggcatacaggatgttgaaccttatgttggggaccctaattcatgtcttcaattgggagcttgaggatggattgagtcatgagaatatggacatgagtgataagtttgggattactatacagaaggctaaacctctccggattattccaatcccaaagtaa

SEQ ID NO: 60 - protein, 502 aa, Lophophora williamsii
MDYCTTLAAILLFLWTCFYFMKLNPFFSKATSTIPLPPGPRPLPIIGNLHQLGERPYCTLADLSRAYGPIISIKFGSITTIVISSSDVAKEMFQKHDLALSSRKVPAAVRANGHDKFSIAWLPVTPKWRYLRKISTVQLFSTQRLDARQSLRQAKVAELLDYVRVCSNVGESVDIGEVAFTTLLNLLSNAFFSLDLASYSSVASGEFKGLVWKIVEEIGKPNLADFFPLLGFISKLSVNRELMGYGNKLNEVFAKIIEERLSAANSSEGRDDGDVLDTLLGLMKEDDSELSLDDIKHLLMDFFTAGTDTTSSTLEWTMTELLHNPEKMSEAEAELERVLGRETLPIQESDIPKLPYLQAVVKETLRMHPPTVFLLPRKADSDVELCGYLVPKNAQVFVNLWAISYDPNTWENPDVFSPERFLDQDVDMKGQDFGFIPFGAGRRMCPGLTLAYRMLNLMLGTLIHVFNWKLEDGLSHENMDMSDKFGITIQKAKPLRIIPIPK

SEQ ID NO: 61 - protein, 501 aa, Lophophora williamsii
MDYYAILAVVLPFLWTCFYLLKLNPTSKLTSTTRLPPGPRPLPIIGNLHQLGERPHRTLANLAKVYGPIISLKFGSITTIVVSSADVAKEMFQKHDLALASRKVPAAVRANGHDNFSVAWLPVTPKWRFLRKISAIQLFSTQRLDARQSLRQAKVVELLDYVKARSSAGEPVDIGEAVFTTSLNLLSNTFFSMNLASYGSAASGEFKELVWKMMEEIGKPNLADCFPLVGFISRMSVNRELMVYGNKLNEVFAEIIEKRLSAANSSKDRGNGDVLDTLLRIMEEDDSELSLDDIMHLLMDFFTAGTDTTSSTLEWAMTELLHNPEKMAKAQAEVEQVLGKDTVLIQESDIAKLPYLQPTVKETLRMHPPTVFLLPRKADSGVELYGHLVPKNAQVFVNLWAISYDPSTWENPDLFSPERFLDQDIDMKGQDFGFIPFGAGRRICPGLTLACRMLNLMLATLIHVFNWKLGDGLSPEDLDMTDKFGITIQKAKPLRAIPILK

SEQ ID NO: 62 - protein, 501 aa, Lophophora williamsii
MDYFPILAFVLPFLWTCFYFLKLNPTSKLTSTIPLPPGPRPLPIIGNLHQLGERPHRTLANLAKVYGPIISLKFGSITTIVVSSADVAKEMFQKHDLALASRKVPAAVRANGHDNFSIAWLPVTPKWRFLRKISAIQLFSTPRLDARQSLRQAKVVELLDYIKARSNAGEPVDIGEAAFTTSLNLLSNTFFSMDLASYSSAASGEFKELVWKIMEEIGKPNLADCFPLVGFISKLSVNRELMGYGNKLNEVFAEIIEKRLSAANSSEGRGNGDVLDTLLRIMEEDDSELSRDDIMHLLMDFFTAGTDTTSSTLEWAMTELLHNPEKMAKAQAELKHVLGKDTVLIEESDISKLPYLQATVKETLRMHPPTVFLLPRKADSDVELYGHLVPKNAQVFVNLWAISYDPSTWENPNLFSPERFLDQDVDMKGQDFGFIPFGAGRRICPGMTLAYRMLNLMLGTLIHVFNWKLGDGLSPEDLDMTDKFGITVQKAKPLRAIPILK

SEQ ID NO: 63 - protein, 505 aa, Lophophora williamsii
MDYYTTLAAILLFLWTCFYFMKRNPFSSTATTSTILLPPGPKPLPIIGNLHQLGGRPYRTLADLARAYGPIISIKFGSVTTIVVSSSDVAKEMFQKHDLALSSRKVPAAVRANGHDNFSIAWLPVTPKWRYLRKISTVQLFSTQRLDARQSLRQAKVAELLDYVRMCSNVGESVDIGEVAFTTQLNLLSNTFFSLDLASYSSVASGEFKELVWKIVQEIGKPNLADFFPCLGFISKLSVNRELMGYGNKLNEGFAKIIEETLSFATSSEGRGDGGGDVLDTLLRLMKEDDSELSLDDIKHLLMDFFTAGTDTTSSTLEWAMAELLHNPEKMSEAQAELERVLGRDTLSIQESDIPKLPYLQAVLKETLRMHPPTVFLLPRKADSDVELCGYLVPKNAQVFVNLWAISYDPNTWENPDVFSPKRFLDQDVDMKGQDFGFIPFGAGRRMCPGLTLAYRMLNLMLGTLIHVFNWELEDGLSHENMDMSDKFGITIQKAKPLRIIPIPK

SEQ ID NO: 64 - protein, 38 aa, Lophophora williamsii
LVDVGGGLGGINFDLPHVEHVGGDMFKGKVIVGGKERT

SEQ ID NO: 65 - protein, 39 aa, Lophophora williamsii
LVDVGGGNGGINFDQPHVEHVGGDIFGGGKVIVGGMERT

SEQ ID NO: 66 - protein, 38 aa, Lophophora williamsii
LVDMGGNRGGINFDLPHVTHVAGDMFHGKVIVNGKERT

SEQ ID NO: 67 - protein, 38 aa, Lophophora williamsii
LVDMGGNRGGINFDLPHVIHVAGDMFHGKVIVNGKERT

SEQ ID NO: 68 - protein, 38 aa, Lophophora williamsii
LVDIAGGTGGINFDLPHVEHVGGDMFHGKVIVGGKERT

SEQ ID NO: 69 - protein, 38 aa, Lophophora williamsii
LVDVGGGTGGINFDLPHVEHIGGDMFHGKVIVGGKERT

SEQ ID NO: 70 - protein, 9 aa, Lophophora williamsii
CILHDWSDE

SEQ ID NO: 71 - protein, 9 aa, Lophophora williamsii
WILHCFGDE

SEQ ID NO: 72 - protein, 9 aa, Lophophora williamsii
NVFHDWDDE

SEQ ID NO: 73 - protein, 9 aa, Lophophora williamsii
WTLCDWSDE

SEQ ID NO: 74 - protein, 9 aa, Lophophora williamsii
WICHDWSDE

SEQ ID NO: 75 - protein, 368 aa, Lophophora williamsii
MGSYHDEKNVMGGSDQGELCSLAMTIASGSVPAMVLKALIELDVLELIKRAGPGAQLSPAQIVAQLPTKNPDAGTMLDRMLRLLASYEILSCSTQTLPDGRVERLYGLAPVCQFFTNNEDGATLGAIVQLSQEKLVVESWYHLKDAVLDGGIPFKKARGMTIYEYPKTDPRFNQVFNSGMSNYSTIIMRDLLERYKGFEGISTLVDIAGGTGASINMVVSKYPTIKGINFDLPHVIKDAPSYDGVEHVGGDMFESVTKADAMLIKWTLCDWSDEHCLKLLKNCYNALPDNGKVIVCDYVLPMAPETSNAAKTVFLLDAIMLANFVGGKGRTKQEFEALARKVGFEGFEVPCSSYDFSVMELFKKKIIK

SEQ ID NO: 76 - protein, 367 aa, Lophophora diffusa
MGSYHDEKNVMGGPDQGDLCSLAMTIASGSVPAMVLKALIELDVLELIKRAGPGAQLSPAQIVAQLPTKNPDAGTMLDRMLRLMGSYGILSCSTQTLPDGRVERLYGLAPVCQFFTKNEDGATLGAIVQLSQEKLVVESWYHLKDAVLDGGIPFKKAHGMTIYEYPKTDPRFNQVFNSGMSNYSTIIMRDLLERYKEFQGISTLVDIAGGTGASINMVVSKYPTIKGINFDLPHVIKDAPSYDGVEHVGGDMFESVTKADAMLIKWTLCDWSDEHCLKLLKTCYNAVPDNGKVIVCDYVLPMAPETSNAAKTVFLLDAIMLANFVGGKGRTKQEFEALARKVGFEGFEVACSSYDFSVMELSKKKNN

(19) United States
(12) Patent Application Publication
(10) Pub. No.: US 2026/0242834 A1
(43) Pub. Date: Aug. 20, 2026

(54) ENZYMES, POLYNUCLEOTIDES ENCODING SAME, AND METHODS OF USING SAME FOR PRODUCING MESCALINE

(71) Applicant: YEDA RESEARCH AND DEVELOPMENT CO. LTD., Rehovot (IL)

(72) Inventors: Asaph AHARONI, Tel Aviv (IL); Paula BERMAN, Rehovot (IL); Sayantan PANDA, Rehovot (IL); Luis Alejandro DE HARO, Rehovot (IL)

(21) Appl. No.: 19/402,875

(22) Filed: Nov. 26, 2025

Related U.S. Application Data
(63) Continuation of application No. PCT/IL2024/050533, filed on May 30, 2024.
(60) Provisional application No. 63/470,200, filed on Jun. 1, 2023.
(60) Provisional application No. 63/471,688, filed on Jun. 7, 2023.
(60) Provisional application No. 63/536,454, filed on Sep. 4, 2023.
(60) Provisional application No. 63/608,321, filed on Dec. 11, 2023.

(51) Int. Cl. C12P 13/00; C12N 1/18; C12N 9/02; C12N 9/10; C12N 9/88

(52) U.S. Cl. CPC C12P 13/001; C12N 1/18; C12N 9/0071; C12N 9/1007; C12N 9/88; C12Y 201/01141; C12Y 401/01025

(57) ABSTRACT

The present invention provides polynucleotide sequences encoding a protein belonging to a decarboxylating (DC) enzyme family and/or a protein belonging to a hydroxylating (CYP) enzyme family. Further provided are a plasmid including the polynucleotide, a transgenic cell, and/or a plant including same. Further provided is a method for synthesizing mescaline.

### CROSS-REFERENCE TO RELATED APPLICATIONS

This application is a Bypass Continuation of PCT Patent Application No. PCT/IL2024/050533 having International filing date of 30 May 2024, which claims the benefit of priority of U.S. Provisional Patent Application No. 63/470,200, titled “O- AND N-METHYL TRANSFERASES FROM LOPHOPHORA WILLIAMSII (PEYOTE)”, filed 1 Jun. 2023, of U.S. Provisional Patent Application No. 63/471,688, titled “L-TYROSINE DECARBOXYLASE FROM LOPHOPHORA WILLIAMSII (PEYOTE)”, filed 7 Jun. 2023, of U.S. Provisional Patent Application No. 63/608,321, titled “ENZYMES, POLYNUCLEOTIDES ENCODING SAME, AND METHODS OF USING SAME FOR PRODUCING MESCALINE”, filed 11 Dec. 2023 and of U.S. Provisional Patent Application No. 63/536,454, titled “CYTOCHROME P450 ENZYMES FROM LOPHOPHORA WILLIAMSII (PEYOTE)”, filed 4 Sep. 2023. The contents of all these applications are incorporated herein by reference in their entirety.

### REFERENCE TO AN ELECTRONIC SEQUENCE LISTING

The contents of the electronic sequence listing (YEDA-P-035-PCT.xml; size: 123,555 bytes; and date of creation: May 15, 2024) is herein incorporated by reference in its entirety.

### FIELD OF INVENTION

The present invention relates to enzymes including polynucleotides encoding same, and methods of using same, such as for producing mescaline.

### BACKGROUND

In recent years, there has been a resurgence of interest in psychedelics due to their reported efficacy in producing long-lasting symptom reduction in phase II clinical trials, offering potential transformative effects on neuroscience and psychiatry as currently understood. The historical use of psychedelic cacti can be traced back nearly 6000 years. Mescaline, the primary hallucinogenic compound found in these cacti, was first isolated and identified by Arthur Heffter in 1897. This 5-HT_{2A} serotonin receptor agonist exerts 1,000-3,000 times less potent effects than LSD and 30 times less potent than psilocybin due to much lower binding affinity. Mescaline is classified as a Schedule I controlled substance and is prohibited in most countries. Nevertheless, Lophophora williamsii (L. williamsii, Peyote) which serves as the primary natural reservoir of mescaline, has obtained legal sanction for ceremonial utilization by the Native American Church as a medium for spiritual connection with the Great Spirit (God).

The biosynthetic pathway of mescaline in Peyote has been postulated for over fifty years, primarily based on radioisotope labeling studies and extensive metabolic profiling. Despite this, the specific enzymes responsible for each step have yet to be elucidated. Additionally, this prediction suggests that some metabolites may be biosynthesized through multiple routes.

### SUMMARY

The present invention, in some embodiments, is based, at least in part, on the identification of four groups of enzymes responsible for five out of six steps required for mescaline biosynthesis. Out of these, the inventors identified two types of magnesium-dependent regiospecific O-methyltransferases (OMTs), and a highly similar methyl transferase (MT) orthologue that does not bind a magnesium ion and non-specifically N-methylates all the phenethyl intermediates from this pathway.

The present invention, in some embodiments, is further based, at least in part, on an engineered stepwise production of mescaline in Nicotiana benthamiana (N. benthamiana) plants using a known hydroxylase from beetroot. The inventors have also identified in two other cacti orthologs of all the active enzymes that shed light on the evolution of this biosynthetic pathway.

According to the first aspect, there is provided an isolated DNA molecule comprising a nucleic acid sequence having at least 85% homology to SEQ ID NO: 1, or any combination thereof. According to another aspect, there is provided an isolated DNA molecule comprising a nucleic acid sequence having at least 80% homology to any one of SEQ ID Nos: 5, 6, 15, or any combination thereof. According to another aspect, there is provided a plasmid or an agrobacterium comprising the isolated DNA molecule of the invention. According to another aspect, there is provided an isolated polypeptide encoded by any one of: (a) the isolated DNA molecule of the invention; and (b) the plasmid or agrobacterium of the invention. According to another aspect there is provided transgenic cell comprising: (a) the isolated DNA molecule of the invention; (b) the plasmid or agrobacterium of the invention; (c) the isolated polypeptide of the invention; or (d) any combination of (a) to (d). According to another aspect, there is provided an extract derived from the transgenic cell of the invention, or any fraction thereof. According to another aspect, there is provided a composition comprising: (a) the isolated DNA molecule of the invention; (b) the plasmid or agrobacterium of the invention; (c) the isolated polypeptide of the invention; (d) the transgenic cell of the invention; (e) the extract of the invention; or (f) any combination of (a) to (e), and an acceptable carrier. According to another aspect, there is provided a method for synthesizing mescaline comprising the steps: (a) providing a cell comprising any one of: i. the isolated DNA molecule of the invention; ii. the plasmid or agrobacterium of the invention; and iii. both (i) and (ii); and (b) culturing the cell from step (a) such that at least one polypeptide encoded by any one of the isolated DNA molecule, the plasmid or agrobacterium, and both, is expressed, thereby synthesizing mescaline or a precursor thereof. According to another aspect, there is provided a method for synthesizing mescaline comprising contacting a mescaline precursor with an effective amount of the isolated polypeptide of the invention, thereby synthesizing mescaline. According to another aspect, there is provided mescaline synthesized according to the method of the invention. According to another aspect, there is provided a composition comprising the mescaline of the invention, and an acceptable carrier.

In some embodiments, the nucleic acid sequence having at least 85% homology to SEQ ID NO: 1 is 1,400 to 1,700 nucleotides long. In some embodiments, the nucleic acid sequence encodes a protein being a decarboxylase. In some embodiments, the nucleic acid sequence having at least 83% homology to any one of SEQ ID Nos: 5, 6, and 15 is 1,450 to 1,650 nucleotides long. In some embodiments, the nucleic acid sequence encodes a protein being a hydroxylase. In some embodiments, the plasmid or agrobacterium further comprises an additional nucleic acid sequence having at least 75% homology to any one of SEQ ID Nos: 44, 48, 53, 54, or any combination thereof. In some embodiments, the additional nucleic acid sequence encodes a protein being a methyltransferase. In some embodiments, the isolated polypeptide comprises an amino acid sequence with at least 97% homology to SEQ ID NO: 28. In some embodiments, the isolated polypeptide comprises an amino acid sequence with at least 96% homology to any one of SEQ ID Nos: 32, 33, and 42. In some embodiments, the isolated polypeptide is characterized by having an activity of releasing a carboxyl group from a substrate, thereby releasing CO₂. In some embodiments, the isolated polypeptide is characterized by having an activity of introducing a hydroxyl group into a substrate. In some embodiments, the transgenic cell is any one of: a unicellular organism, a cell of a multicellular organism, and a cell in a culture. In some embodiments, the unicellular organism comprises a fungus or a bacterium. In some embodiments, the fungus is a yeast cell. In some embodiments, the extract comprises the isolated DNA molecule, the isolated protein, or both. In some embodiments, the at least one polypeptide is characterized by having an activity selected from the group consisting of: releasing a carboxyl group from a substrate, introducing a hydroxyl group into a substrate, transferring a methyl group from a donor molecule to a substrate, and any combination thereof. In some embodiments, the culturing comprises supplementing the cell with an effective amount of a mescaline precursor selected from the group consisting of: L-tyrosine, L-DOPA, Dopamine, 3-MeO-tyramine, 3,4-diOH-5-MeO-phenethylamine, 4-OH-3,5-diMeO-phenethylamine, and any combination thereof. In some embodiments, the cell is a prokaryote cell or a eukaryote cell. In some embodiments, the cell is a transgenic cell or a cell transfected with the isolated DNA molecule of the invention or the plasmid or agrobacterium of the invention. In some embodiments, the method further comprises a step before step (a), comprising introducing or transfecting the cell with the plasmid or agrobacterium. In some embodiments, the method further comprises contacting the mescaline precursor with an isolated polypeptide comprising an amino acid sequence having at least 85% homology to any one of SEQ ID Nos: 44, 48, 53, 54, and any combination thereof. In some embodiments, the mescaline precursor is selected from the group consisting of L-tyrosine, L-DOPA, dopamine, 3-MeO-tyramine, 3,4-diOH-5-MeO-phenethylamine, 4-OH-3,5-diMeO-phenethylamine, and any combination thereof. In some embodiments, the contacting is in a cell-free system. In some embodiments, the acceptable carrier is a pharmaceutically acceptable carrier. In some embodiments, the composition is a pharmaceutical composition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B include a predicted biosynthetic pathway of mescaline 1 and related phenethylamines in Peyote as well as structures of tetrahydroisoquinoline (THIQ) alkaloids. (1A) The active enzymes identified in this study are marked by their names. TyDC, L-tyrosine/L-DOPA decarboxylase; CYP, cytochrome P450; PPO, polyphenol oxidase; OMT, O-methyltransferase; NMT, N-methyltransferase. Putative types of enzymes appear in black. According to the predicted pathway, some metabolites may be biosynthesized in more than one route as depicted by the main molecules marked in yellow, orange, and blue according to modules I-1II, respectively. Decarboxylation, hydroxylation, O-methylation, and N-methylation reactions are marked in pink, red, blue, and green, respectively. (1B) Chemical structures of several known tetrahydroisoquinolines (TIQs) from Peyote putatively assigned according to LC-MS/MS fragmentation (FIG. 13).

FIGS. 2A-2K include a table, photographs, a chromatograph, and micrographs showing that mescaline 1 accumulates mostly in the Peyote button sub-epidermal green tissue. (2A) Absolute quantification of mescaline 1 in different tissues of Peyote and San Pedro cacti (% w/w per fresh weight, mean±s.d.; n=3). The tissues used for the analysis are annotated on images (2b, 2d, and 2E). (2C) Extracted ion current chromatograms of m/z 212.128 Da from a Peyote button extract versus mescaline 1 standard. (2D) Horizontal and (2E) lateral sections of Peyote were analyzed using matrix-assisted laser desorption/ionization-mass spectrometry imaging (MALDI-MSI; n=2) of m/z (2F-2G) 212.128±0.01 Da which corresponds mainly to the protonated masses of mescaline 1, N-methyl-4-hydroxy-3,5-dimethoxy-phenethylamine 20, and N-methyl-3-hydroxy-4,5-dimethoxy-phenethylamine 22 (as shown in 2C); (2H-2I) 893.545±0.01 Da which corresponds to chllorophyl b; and (2J-2K) 166.123±0.01 Da which corresponds to hordenine 13. Skin (the sub-epidermal green area) and flesh tissues are marked on the optical images (2D-2E) of Peyote sections.

FIGS. 3A-3B include a phylogenetic tree, heatmaps, and photographs showing that taxonomically distant cacti produce mescaline 1. (3A) Phylogenetic relationship of the cactoideae family and total ion current chromatograms of Lophophora diffusa (L. diffusa, false Peyote) skin, Lophophora williamsii (Peyote) skin and Echinopsis pachanoi (San Pedro) spines show that Peyote and San Pedro, two taxonomically distant cacti (cacteae and trichocereae, respectively) produce mescaline 1, while L. diffusa, a taxonomically close relative of Peyote (both cacteaea), contains none. Representative photographs of each species appear next to the corresponding chromatogram. Major identified metabolites are marked on each chromatogram. Due to the limited availability of molecular data, this proposed phylogenetic hypothesis relies on a synthesis of existing literature and represents a preliminary interpretation of the relationships among the taxa under study. (3B) Differential analysis of identified phenethylamines in different tissues of Peyote, L. diffusa and San Pedro. Peak areas (z score of mean, n=3) were used for the comparisons. Peaks that were not detected appear in white. Metabolites were identified as described in FIGS. 11-12. Main phenethylamines from the mescaline 1 biosynthetic pathway are marked in red and their N-methylated products appear below on the same square.

FIGS. 4A-4C include graphs and a scheme showing genome assembly and annotation of Peyote. (4A) Longest representative contigs containing the candidate genes evaluated in this work (scale: 100 Kb). Colored names denote the most active functionally characterized enzymes in the mescaline 1 pathway. (4B) BUSCO completeness metrics of the genome assembly, the Isoseq and Illumina-based transcriptomes. (4C) Two tandem metabolic gene clusters of O-methyl-transferases. Blue and pink colors mark the corresponding clusters in 4A. LwOMT1 and LwOMT5 are highlighted as they resulted in the most active variants.

FIGS. 5A-5F include a heatmap, graphs and curves showing functional characterization of Peyote L-tyrosine/L-DOPA decarboxylase (TyDC). (5A) Gene expression of the putative TyDCs observed in Peyote tissues (z score, n=3). (5B) Phylogenetic analysis of putative TyDC proteins from Peyote and functionally characterized enzymes from other plants. The functional LwTyDC2 is highlighted in pink. Comparison of ion abundance peak areas (mean±s.d.; n=3-4 biological replicates) following (5C) transient expression of LwTyDC2, BvCYP76AD6 and their combinations in Nicotiana benthamiana leaves, and (5D) in vitro assays using lysates from E. coli cells transformed with TyDC candidates, utilizing either L-tyrosine 2 or L-DOPA 5 as substrates in the presence of pyridoxal 5-phosphate. (5E) Extracted ion chromatograms of in vitro assays following protein purification of LwTyDC2. (5F) Matching of MS/MS spectra for the identified tyramine 4 and dopamine 3 alongside their corresponding standards. All the metabolites were identified by exact mass, retention time (5E) and MS/MS spectra (5F). EV, empty vector.

FIGS. 6A-6D include graphs, curves, and synthesis scheme showing functional characterization of Peyote CYPs in Nicotiana benthamiana. (6A) Ion abundance peak areas (mean±s.d.; n=4 biological replicates) following transient expression of CYP11 and CYP12 (LwCYP12) in N. benthamiana leaves. LwCYP12 catalyzes the hydroxylation (marked in red) of both tyramine 4 to dopamine 3 and 3-MeO-tyramine 7 to 3,4-dihydroxy-5-methoxy-phenethylamine 6. All the metabolites were identified by exact mass, retention time and MS/MS spectra. EV, empty vector. (6B) Scheme showing the reactions catalyzed by LwCYP12 and an unknown O-methyltransferase from N. benthamiana (NbOMT). The reactions catalyzed by other functionally characterized members of the CYP76AD subfamily are shown for reference. (6C) Phylogenetic analysis of the CYP76AD-subfamily within betalain-producing Caryophyllales showing three distinct clades (α, β and γ). Functionally characterized enzymes in a-(CYP76AD1/3), β-(CYP76AD5/6/15) and y-clades (LwCYP12) appear on 6B and 6C in green, yellow, and red, respectively. (6D) LC-MS extracted ion chromatograms of 3,4-dihydroxy-5-methoxy-phenethylamine 6 and 4-hydroxy-3,5-dimethoxy-phenethylamine 9 following transient expression of CYP2 (LwCYP2) and CYP3 in N. benthamiana leaves with supplementation of 100 μg ml⁻¹ of 3-MeO-tyramine 7. All the metabolites were identified by exact mass, retention time and MS/MS spectra. EV, empty vector.

FIGS. 7A-7G include heatmaps and curves showing functional characterization of Peyote methyltransferases (MTs). (7A) Gene expression of the putative MTs and O-methyltransferases (OMTs) in Peyote tissues (z score, n=3). Heatmaps of ion abundance peak areas (mean, n=3) following transient expression in N. benthamiana leaves of (7B) a mixture of MT candidates from Peyote (MT1-MT4) with and without supplementation of 3,4-dihydroxy-5-methoxy-phenethylamine 6; (7C) individual MTs; and (7D) only MT2 (LwNMT1) following supplementation of 3,4-dihydroxy-5-methoxy-phenethylamine 6, 4-hydroxyl-3,5-dimethoxy-phenethylamine 9 or mescaline 1. Low-abundance contaminations from the standards are marked with asterisks. In planta O-methylation activities (3→7, 6→9 and 6→10) were attributed to one and/or several endogenous OMTs from N. benthamiana (NbOMTs) since these products were also observed in plants infiltrated with an empty vector (EV). A single N-methyltransferase from Peyote (LwNMT1) was identified with promiscuous activity (positive N-methylated products are marked with “+”). Metabolites used for feeding are highlighted in yellow and their structures are shown for reference. LC-MS extracted ion chromatograms and metabolite peak areas of lysates from E. coli transformed with OMT1-OMT11 candidate enzymes in the presence of S-adenosyl-L-methionine and either (7E) dopamine 3, (7F) 3,4-dihydroxy-5-methoxy-phenethylamine 6, or (7G) 4-hydroxy-3,5-dimethoxy-phenethylamine 9. Products in each assay were identified according to analytical standards and/or extract from a Peyote skin sample. The chemical structure of substrates are shown for reference. The active LwOMT1,5 which O-methylate the meta hydroxyls in mescaline 1, and LwOMT10,11 which work on the para hydroxyl are marked in blue and purple, respectively. Positive O-methylated products are marked with “+”. Low-abundance contaminations from the standards are marked with asterisks. EV, empty vector.

FIGS. 8A-8D include structure analysis showing that docking of 4-diOH-5-MeO-phenethylamine 6 in LwNMT1, LwOMT1 and LwOMT11 reveal specificity of methylation sites. (8A) Regiospecific methylation sites of 4-diOH-5-MeO-phenethylamine 6 by the specific enzymes according to functional characterization. Reagents in the active site of (8B) LwNMT1, (8C) LwOMT1, and (8D) LwOMT11. SAM appears in green (thick tubes), 4-diOH-5-MeO-phenethylamine 6 is cyan (ball-and-stick) and the magnesium ion is magenta. Magnesium-coordinated amino acids are depicted as thin tubes.

FIGS. 9A-9B include graphs and an illustration showing the engineering of the mescaline pathway in N. benthamiana. Transient expression in N. benthamiana of (9A) the functionally characterized enzymes from Peyote; and (9B) only LwOMT11 with supplementation of 4-hydroxy-3,5-dimethoxy-phenethylamine 9. Peak areas of ion abundance were used for the comparisons (mean±s.d.; n=4-5 biological replicates). The identity of peaks was verified on a high-resolution LC-MS by exact mass, retention time and MS/MS spectra. The boxes in (9A) indicate biosynthetic genes that are included in each experiment; full boxes indicate the last added enzyme in a set of co-expressed genes. EV, empty vector.

FIGS. 10A-10E include a graph, a phylogenetic analysis, and heatmaps showing the presence of orthologue enzymes of the mescaline pathway in L. williamsii (peyote), L. diffussa (false peyote) and E. pachanoi (San Pedro). (10A) BUSCO completeness metrics of L. diffussa and E. pachanoi Illumina-based transcriptomes. (10B) Phylogenetic trees of the three cacti constructed with whole-proteome sequences (CITE Prot-SpaM). Phylogenetic reconstruction of orthogroups of the active enzymes (11C) L-tyrosine/L-DOPA decarboxylase (TyDC), (11D) methyl-transferases (MTs), and (11E) cytochrome P450 (CYP). In the case of (11D) different colors represent different orthogroups, but all enzymes were re-aligned and displayed together for comparison.

FIGS. 11A-11J include spectra chemical structures showing the identification of major phenethylamines from the mescaline pathway by analytical standards. (11A-11J) MS/MS spectra matching of phenethylamine standards versus Peyote skin extract. The metabolites were identified according to retention time, accurate mass, and MS/MS spectra. (11E) L-DOPA 5 was not observed in any of the Peyote tissues. 3-OH-4,5-diMeO-phenethylamine 10 was identified according to similar accurate mass and MS/MS fragmentation as 4-OH-3,5-diMeO-phenethylamine 9 (11I versus 11J). The two metabolites eluted from the column at different retention times according to the position of the free hydroxyl.

FIGS. 12A-12G include spectra showing putative identification of N-methylated phenethylamines (12-25) in a Peyote skin extract. (12A-12G) The metabolites were identified by similar fragmentation patterns as the non-N-methylated metabolites (marked in red) with a shift in accurate mass corresponding to one or two methyl groups and increase in retention time with a greater number of methyl groups. Typical fragments corresponding to N-methyl and NN-methyl fragmented groups (C₂H₆N and C₃H₈N, respectively) were observed for most metabolites. (12D) 4-Methoxy tyramine 11 for which the inventors did not have an analytical standard was identified by similar accurate mass and fragmentation pattern as 3-MeO-tyramine 7. The two metabolites eluted from the column at different retention times according to the position of the free hydroxyl.

FIGS. 13A-13F include spectra and chemical structures showing putative identification of major tetrahydroisoquinolines (THIQs 26-31) in a Peyote skin extract. (13A-13F) MS/MS spectra of the putatively identified THIQs. The spectra were arranged by similarity of chemical structures versus fragmentation profiles. (13C) Anhalamine 26, (13B) anhalonidine 28 and (13F) pellotine 30 are major metabolites in Peyote and were putatively assigned by peak heights in the total ion chromatogram (FIG. 3A), accurate mass and MS/MS fragmentation patterns that correspond with the chemical structures of these metabolites. (13A) Anhalonine 27 and (13E) lophophorine 29 which contain a methylenedioxy group, were assigned according to similar fragmentation patterns as the parent (28→27 and 30→29) with a mass shift of two hydrogens and increased retention time for the metabolites with the methylenedioxy group. THIQs (13F) 30 and (13E) 29 showed the typical fragments that correspond with the loss of an N-methyl group (C₂H₆N, 44.05 Da). (13D) Peyophorine 31 had a similar fragmentation pattern as that of lophophorine 29 apart from the parent mass and the fragment 58.06 Da which correspond to the N-ethyl group (C₃H₈N).

FIG. 14 includes images showing matrix-assisted laser desorption/ionization-mass spectrometry imaging (MALDI-MSI) of a Peyote button horizontal section. The protonated m/z ([M+H]⁺±0.01 Da) and corresponding potential phenethylamines appear above each image. The potential phenethylamines were in accordance with LC-MS data of this tissue.

FIG. 15 includes images of matrix-assisted laser desorption/ionization-mass spectrometry imaging (MALDI-MSI) of a Peyote lateral section. The protonated m/z ([M+H]⁺± 0.01 Da) and corresponding potential phenethylamines appear above each image. The potential phenethylamines were in accordance with LC-MS data of this tissue.

FIG. 16 includes a heatmap showing differential analysis of identified tetrahydroiosquinolines (THIQs) in different tissues of Peyote, L. diffusa and San Pedro. Peak areas (z score of mean, n=3) were used for the comparisons. Peaks that were not detected appear in white. THIQs putatively assigned by MS/MS fragmentations (26-31, FIG. 13) and several possible additional THIQs with similar masses and MS/MS products were annotated for differential analysis.

FIGS. 17A-17B include a predicted biosynthetic pathway of mescaline 1 and related phenethylamines in San Pedro according to metabolic profiling. (17A) Phenethylamines that possibly participate in this pathway but were not observed in any of the analyzed tissues appear blurry. Some metabolites in the predicted pathway may be biosynthesized in more than one way as depicted by the main molecules marked in yellow, orange and blue according to modules I-III, respectively. Decarboxylation, hydroxylation, O-methylation and N-methylation reactions are marked in pink, red, blue and green, respectively. TyDC, L-tyrosine/L-dopa decarboxylase; CYP, cytochrome P450; OMT, O-methyltransferase; NMT, N-methyltransferase. (17B) Chemical structure of anhalamine 26, the only tetrahydroisoquinoline (THIQ) putatively identified by LC-MS/MS fragmentation (FIG. 13) in Peyote, that was also present in San Pedro.

FIGS. 18A-18B include a predicted biosynthetic pathway of mescaline 1 and related phenethylamines in L. diffusa according to metabolic profiling. (18A) Phenethylamines that possibly participate in this pathway but were not observed in any of the analyzed tissues appear blurry. Some metabolites in the predicted pathway may be biosynthesized in more than one way as depicted by the main molecules marked in yellow, orange and blue according to modules I-III, respectively. Decarboxylation, hydroxylation, O-methylation and N-methylation reactions are marked in pink, red, blue and green, respectively. TyDC, L-tyrosine/L-dopa decarboxylase; CYP, cytochrome P450; OMT, O-methyltransferase; NMT, N-methyltransferase. (18B) Chemical structures of three tetrahydroisoquinolines (THIQs) putatively identified by LC-MS/MS fragmentation (FIG. 13) in Peyote, that were also present in L. diffusa.

FIGS. 19A-19B include graphs showing that LwTyDC2 exhibits specific in vitro selectivity towards L-tyrosine 2 when both L-tyrosine 2 and L-DOPA 5 are present as substrates. Comparison of ion abundance peak areas (mean±s.d.; n=3-4 biological replicates) of (19A) L-tyrosine 2 and (19B) tyramine 4 from in vitro assays using lysates from E. coli cells transformed with TyDC candidates and L-DOPA 5 as the substrate. L-tyrosine 2 was identified in all the samples probably originating from the E. coli lysates, while the production of tyramine 4 in addition to a sharp reduction in L-tyrosine 2 was observed only with LwTyDC2. All the metabolites were identified by exact mass, retention time and MS/MS spectra. EV, empty vector.

FIGS. 20A-20D include a heatmap, and graphs showing functional characterization of Peyote CYPs in N. benthamiana. (20A) Gene expression of the selected candidate CYPs observed in Peyote tissues (z score, n=3). (20B) Phylogenetic analysis of the putative CYP proteins from Peyote (marked in orange) and functionally characterized enzymes from other plants. Enzymes that hydroxylate metabolites with structural similarities to L-tyrosine 2 and 3-MeO-tyramine 7 are marked in blue, and their regioselective reactions are shown as reference in (20C). Functional CYPs were highlighted in red. (20D) Comparison of ion abundance peak areas (mean±s.d.; n=3 biological replicates) of tyramine 4, dopamine 3 and 3-MeO-tyramine 7 following transient expression of CYP and PPO candidates in Nicotiana benthamiana leaves. No significant differences were observed for these metabolites.

FIGS. 21A-21B include phylogenetic analysis and extracted ion chromatograms. (21A) Phylogenetic analysis of MT protein candidates from Peyote (appear in color) and O-methyltransferases (OMTs) and N-methyltransferases (NMTs) from other plants. The selection of the proteins was based on functionally characterized MT enzymes. The assignment to clades was according to Lam et al. The branches of functionally characterized NMTs from the literature are marked in green. The active NMT and OMT enzymes from Peyote appear in green, blue, or purple, respectively. LwOMT1, LwOMT5 and LwNMT1 clade with hydroxycinnamic acid (HCA) OMTs, and LwOMT10 and LwOMT11 cluster with o-dihydric phenols. The Maximum Likelihood tree was constructed with 300 bootstrap tests based on a MUSCLE multiple alignment using the MEGA11 software. The evolutionary distances were computed using the JTTmatrix-based method. (21B) Extracted ion chromatograms of N,N-dimethyl-mescaline 25 produced in N. benthamiana following transient expression of LwNMT1 and supplementation of N-methyl-mescaline 24. EV, empty vector.

FIGS. 22A-22B include sequence alignment of methyltransferases (MTs) and extracted ion chromatograms. (22A) Amino acid sequence alignment of MT protein candidates from Peyote showing conserved regions (I-IV) of OMTs according to the literature. The diagram showing the relative positions of the OMT consensus regions (bottom) was adapted from Ibrahim et al. (1998). (22B) LC-MS extracted ion chromatograms of purified LwOMT1 and LwOMT11 enzymes in the presence of S-adenosyl-L-methionine and either dopamine 3, 3,4-dihydroxy-5-methoxy-phenethylamine 6, or 4-hydroxy-3,5-dimethoxy-phenethylamine 9. Products in each assay were identified according to analytical standards and/or extract from a Peyote skin sample. The chemical structure of substrates are shown for reference. The active sites and chromatograms of LwOMT1 and LwOMT11 are marked in blue and purple, respectively. EV, empty vector. In 22A presented are the sequences of: LwOMT10, LwOMT11, and OMT12 as set forth in SEQ ID NO: 64; OMT4, and OMT9 as set forth in SEQ ID NO: 65, OMT2, OMT6, OMT8, and OMT7 as set forth in SEQ ID NO: 66; OMT3 as set forth in SEQ ID NO: 67; LwNMT1 as set forth in SEQ ID NO: 68; and LwOMT1, and LwOM5 as set forth in SEQ ID NO: 69.

FIGS. 23A-23B include three-dimensional (3D) structures and multiple sequence alignment showing comparison of LwOMT1 (grey wire) and LwNMT1 (orange wire) 3D structures. (23A) The SAM (green, thick tubes) and 3,4-diOH-5-MeO-phenethylamine 6 (cyan, thick tubes) molecules were positioned for 3-O-methylation. As shown, F324 (depicted as thick orange tubes) present in LwNMT1 forms steric clashes with the poly-substituted benzene “head” hindering the proximity of SAM from the OH-groups of the substrate for effective methylation. H268 of LwOMT1 (depicted as faded plum thick tubes) is missing in LwNMT1 which was predicted to be essential for the reaction complex formation reinforcing the lack of a magnesium ion in this center. Y163 and C269 of LwNMT1 are shown as thin tubes. (23B) Sequence alignment of all MT candidates showing the missing histidine in LwNMT1 (H268 in LwOMT1 and H292 in LwOMT11). In 23B presented are the sequences of: LwOMT10, LwOMT11, and OMT12 as set forth in SEQ ID NO: 70; OMT4, and OMT9 as set forth in SEQ ID NO: 71, OMT2, OMT6, OMT8, OMT3, and OMT7 as set forth in SEQ ID NO: 72; LwNMT1 as set forth in SEQ ID NO: 73; and LwOMT1, and LwOM5 as set forth in SEQ ID NO: 74.

FIGS. 24A-24C include graphs and spectra showing that hydroxycinnamic acid amides (HCAAs) and phenethylamines compete over the same enzymes and intermediates in N. benthamiana. (24A) Extracted ion chromatograms, (24B) MS/MS spectra and chemical structures, and (24C) peak areas of HCAAs in N. benthamiana as a result of the stepwise co-expression of the functionally characterized enzymes from Peyote along with BVCYP76AD6. Samples were analyzed on a high-resolution LC-MS. HCAAs 32-35 were identified according to analytical standards and the rest were putatively assigned according to accurate mass and typical fragmentation patterns according to Li et al. (2018). Several metabolites had similar masses and MS/MS spectra and were therefore annotated as put1/2. Metabolite 38 (UNK344) had a similar mass and fragmentation pattern as N-feruloyl-3-MeO-tyramine. Metabolite 39 was putatively assigned as N-feruloyl-5-methoxyyramine. Peak areas of ion abundance in (23C) were used for the comparisons (mean±s.d.; n=3-5 biological replicates). The boxes indicate biosynthetic genes that are included in each experiment; full boxes indicate the last added enzyme in a set of co-expressed genes. EV, empty vector.

FIG. 25 includes a sequence alignment analysis of LwOMT1 (SEQ ID NO: 44), LwOMT5 (SEQ ID NO: 48) and LwNMT1 (SEQ ID NO: 75) from Peyote and the orthologue LdNMT1 (SEQ ID NO: 76) from Lophophora diffusa. The missing histidine (H268 in LwOMT1 and LwOMT5) and presence of the bulky phenylalanine (F324 in LwNMT1 and LdNMT1) in the two NMTs are highlighted in yellow. These results suggest a similar mechanism of N-methylation for LdNMT1 as previously described.

### DETAILED DESCRIPTION

The present invention, in some embodiments, is directed to polynucleotides including isolated protein encodes by same, that are involved in production/or synthesis of psychedelics, such as mescaline, and methods of using same in the production or synthesis of mescaline.

According to some embodiments, there is provided a polynucleotide comprising a nucleic acid sequence comprising any one of SEQ ID Nos.: 1-4, or any combination thereof.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence set forth in SEQ ID NO: 1. In some embodiments, the polynucleotide comprises a nucleic acid sequence with at least 83%, at least 85%, at least 87%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 1. Each possibility represents a separate embodiment of the invention.

In some embodiments, the polynucleotide comprises or consists of the nucleic acid sequence set forth in SEQ ID NO: 2. In some embodiments, the polynucleotide comprises a nucleic acid sequence with at least 83%, at least 85%, at least 87%, at least 95%, or at least 99% homology or identity to SEQ ID NO: 2, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 2. Each possibility represents a separate embodiment of the invention.

In some embodiments, the polynucleotide comprises or consists of the nucleic acid sequence set forth in SEQ ID NO: 3. In some embodiments, the polynucleotide comprises a nucleic acid sequence with at least 83%, at least 85%, at least 87%, at least 95%, or at least 99% homology or identity to SEQ ID NO: 3, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 3. Each possibility represents a separate embodiment of the invention.

In some embodiments, the polynucleotide of the invention comprises 1,400 to 1,700 nucleotides. In some embodiments, the polynucleotide of the invention is 1,400 to 1,700 nucleotides long. In some embodiments, 1,400 to 1,700 nucleotides comprises: at least 1,400 nucleotides, at least 1,500 nucleotides, at least 1,600 nucleotides, at least 1,650 nucleotides, at least 1,450 nucleotides, at least 1,550 nucleotides, or at least 1,490 nucleotides, or any value and range therebetween. Each possibility represents a separate embodiment of the invention. In some embodiments, 1,400 to 1,700 nucleotides comprises: 1,400 to 1,450 nucleotides, 1,500 to 1,575 nucleotides, 1,400 to 1,650 nucleotides, 1,600 to 1,700 nucleotides, 1,450 to 1,650 nucleotides, 1,420 to 1,590 nucleotides, or 1,410 to 1,690 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, or at least 3 different nucleic acid sequences, or any value and range therebetween, wherein each of the different nucleic acid sequences is selected from SEQ ID Nos: 1-3. Each possibility represents a separate embodiment of the invention. In some embodiments, the polynucleotide comprises 1-2, 1-3, or 2-3 different nucleic acid sequences, wherein each of the different nucleic acid sequences is selected from SEQ ID Nos: 1-3. In some embodiments, the polynucleotide comprises a plurality of polynucleotide molecules, wherein each of the plurality of the polynucleotide molecules comprises a different nucleic acid sequence, and wherein the different nucleic acid sequences are selected from SEQ ID Nos.: 1-3. In some embodiments, the polynucleotide encodes a protein characterized by decarboxylating activity. In some embodiments, the polynucleotide encodes a protein characterized by having an activity of releasing a carboxyl group from a substrate, e.g., releasing CO₂. In some embodiments, the polynucleotide encodes a protein being a decarboxylase. In some embodiments, the decarboxylase is a decarboxylase derived from a cactus. In some embodiments, the cactus is a peyote. In some embodiments, the polynucleotide encoding a decarboxylating protein is selected from SEQ ID Nos: 1-3, or an analog thereof having at least 70%, 80%, 90%, or 95% homology or identity thereto.

In some embodiments, the polynucleotide comprises or consists of the nucleic acid sequence set forth in SEQ ID NO: 4. In some embodiments, the polynucleotide comprises a nucleic acid sequence with at least 83%, at least 85%, at least 87%, at least 95%, or at least 99% homology or identity to SEQ ID NO: 4, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 4. Each possibility represents a separate embodiment of the invention.

In some embodiments, the polynucleotide comprises or consists of the nucleic acid sequence set forth in SEQ ID NO: 5. In some embodiments, the polynucleotide comprises a nucleic acid sequence with at least 83%, at least 85%, at least 87%, at least 95%, or at least 99% homology or identity to SEQ ID NO: 5, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 5. Each possibility represents a separate embodiment of the invention.

In some embodiments, the polynucleotide comprises or consists of the nucleic acid sequence set forth in SEQ ID NO: 6. In some embodiments, the polynucleotide comprises a nucleic acid sequence with at least 83%, at least 85%, at least 87%, at least 95%, or at least 99% homology or identity to SEQ ID NO: 6, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 6. Each possibility represents a separate embodiment of the invention.

In some embodiments, the polynucleotide comprises or consists of the nucleic acid sequence set forth in SEQ ID NO: 7. In some embodiments, the polynucleotide comprises a nucleic acid sequence with at least 83%, at least 85%, at least 87%, at least 95%, or at least 99% homology or identity to SEQ ID NO: 7, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 7. Each possibility represents a separate embodiment of the invention.

In some embodiments, the polynucleotide comprises or consists of the nucleic acid sequence set forth in SEQ ID NO: 8. In some embodiments, the polynucleotide comprises a nucleic acid sequence with at least 83%, at least 85%, at least 87%, at least 95%, or at least 99% homology or identity to SEQ ID NO: 8, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 8. Each possibility represents a separate embodiment of the invention.

In some embodiments, the polynucleotide comprises or consists of the nucleic acid sequence set forth in SEQ ID NO: 9. In some embodiments, the polynucleotide comprises a nucleic acid sequence with at least 83%, at least 85%, at least 87%, at least 95%, or at least 99% homology or identity to SEQ ID NO: 9, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 9. Each possibility represents a separate embodiment of the invention.

In some embodiments, the polynucleotide comprises or consists of the nucleic acid sequence set forth in SEQ ID NO: 10. In some embodiments, the polynucleotide comprises a nucleic acid sequence with at least 83%, at least 85%, at least 87%, at least 95%, or at least 99% homology or identity to SEQ ID NO: 10, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 10. Each possibility represents a separate embodiment of the invention.

In some embodiments, the polynucleotide comprises or consists of the nucleic acid sequence set forth in SEQ ID NO: 11. In some embodiments, the polynucleotide comprises a nucleic acid sequence with at least 83%, at least 85%, at least 87%, at least 95%, or at least 99% homology or identity to SEQ ID NO: 11, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 11. Each possibility represents a separate embodiment of the invention.

In some embodiments, the polynucleotide comprises or consists of the nucleic acid sequence set forth in SEQ ID NO: 12. In some embodiments, the polynucleotide comprises a nucleic acid sequence with at least 83%, at least 85%, at least 87%, at least 95%, or at least 99% homology or identity to SEQ ID NO: 12, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 12. Each possibility represents a separate embodiment of the invention.

In some embodiments, the polynucleotide comprises or consists of the nucleic acid sequence set forth in SEQ ID NO: 13. In some embodiments, the polynucleotide comprises a nucleic acid sequence with at least 83%, at least 85%, at least 87%, at least 95%, or at least 99% homology or identity to SEQ ID NO: 13, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 13. Each possibility represents a separate embodiment of the invention.

In some embodiments, the polynucleotide comprises or consists of the nucleic acid sequence set forth in SEQ ID NO: 14. In some embodiments, the polynucleotide comprises a nucleic acid sequence with at least 83%, at least 85%, at least 87%, at least 95%, or at least 99% homology or identity to SEQ ID NO: 14, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 14. Each possibility represents a separate embodiment of the invention.

In some embodiments, the polynucleotide comprises or consists of the nucleic acid sequence set forth in SEQ ID NO: 15. In some embodiments, the polynucleotide comprises a nucleic acid sequence with at least 83%, at least 85%, at least 87%, at least 95%, or at least 99% homology or identity to SEQ ID NO: 15, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 15. Each possibility represents a separate embodiment of the invention. In some embodiments, LwCYP12, as disclosed herein, comprises or consists of the nucleic acid sequence set forth in SEQ ID NO: 15.

In some embodiments, the polynucleotide comprises or consists of the nucleic acid sequence set forth in SEQ ID NO: 56. In some embodiments, the polynucleotide comprises a nucleic acid sequence with at least 83%, at least 85%, at least 87%, at least 95%, or at least 99% homology or identity to SEQ ID NO: 56, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 56. Each possibility represents a separate embodiment of the invention.

In some embodiments, the polynucleotide comprises or consists of the nucleic acid sequence set forth in SEQ ID NO: 57. In some embodiments, the polynucleotide comprises a nucleic acid sequence with at least 83%, at least 85%, at least 87%, at least 95%, or at least 99% homology or identity to SEQ ID NO: 57, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 57. Each possibility represents a separate embodiment of the invention.

In some embodiments, the polynucleotide comprises or consists of the nucleic acid sequence set forth in SEQ ID NO: 58. In some embodiments, the polynucleotide comprises a nucleic acid sequence with at least 83%, at least 85%, at least 87%, at least 95%, or at least 99% homology or identity to SEQ ID NO: 58, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 58. Each possibility represents a separate embodiment of the invention.

In some embodiments, the polynucleotide comprises or consists of the nucleic acid sequence set forth in SEQ ID NO: 59. In some embodiments, the polynucleotide comprises a nucleic acid sequence with at least 83%, at least 85%, at least 87%, at least 95%, or at least 99% homology or identity to SEQ ID NO: 59, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 59. Each possibility represents a separate embodiment of the invention.

In some embodiments, the polynucleotide of the invention comprises 1,400 to 1,700 nucleotides. In some embodiments, the polynucleotide of the invention is 1,400 to 1,700 nucleotides long. In some embodiments, 1,400 to 1,700 nucleotides comprises: at least 1,400 nucleotides, at least 1,500 nucleotides, at least 1,600 nucleotides, at least 1,650 nucleotides, at least 1,450 nucleotides, at least 1,550 nucleotides, or at least 1,490 nucleotides, or any value and range therebetween. Each possibility represents a separate embodiment of the invention. In some embodiments, 1,400 to 1,700 nucleotides comprises: 1,400 to 1,450 nucleotides, 1,500 to 1,575 nucleotides, 1,400 to 1,650 nucleotides, 1,600 to 1,700 nucleotides, 1,450 to 1,650 nucleotides, 1,420 to 1,590 nucleotides, or 1,410 to 1,690 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 5, at least 7, at least 10, or at least 11 different nucleic acid sequences, or any value and range therebetween, wherein each of the different nucleic acid sequences is selected from SEQ ID Nos: 4-15, and 56-59. Each possibility represents a separate embodiment of the invention. In some embodiments, the polynucleotide comprises 1-12, 2-12, 4-12, 6-12, 8-12, 10-12, 2-6, 1-3, or 2-3 different nucleic acid sequences, wherein each of the different nucleic acid sequences is selected from SEQ ID Nos: 4-15, and 56-59. In some embodiments, the polynucleotide comprises a plurality of polynucleotide molecules, wherein each of the plurality of the polynucleotide molecules comprises a different nucleic acid sequence, and wherein the different nucleic acid sequences are selected from SEQ ID Nos.: 4-15, and 56-59. In some embodiments, the polynucleotide encodes a protein characterized by hydroxylating activity. In some embodiments, the polynucleotide encodes a protein characterized by having an activity of introducing a hydroxyl group to a substrate. In some embodiments, the polynucleotide encodes a protein being a hydroxylase. In some embodiments, the encoded protein belongs to the Cytochrome P450 (CYP) family. In some embodiments, hydroxylase is a hydroxylase derived from a cactus. In some embodiments, the cactus is apeyote. In some embodiments, the polynucleotide encoding a hydroxylating protein is selected from SEQ ID Nos: 4-15, and 56-59, or an analog thereof having at least 70%, 80%, 90%, or 95% homology or identity thereto.

According to some embodiments, there is provided an artificial nucleic acid molecule comprising the polynucleotide disclosed herein. In some embodiments, the artificial vector further comprises an additional nucleic acid sequence or an additional polynucleotide.

In some embodiments, the additional polynucleotide comprises or consists of the nucleic acid sequence set forth in SEQ ID NO: 16. In some embodiments, the additional polynucleotide comprises a nucleic acid sequence with at least 83%, at least 85%, at least 87%, at least 95%, or at least 99% homology or identity to SEQ ID NO: 16, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 16. Each possibility represents a separate embodiment of the invention.

In some embodiments, the additional polynucleotide comprises or consists of the nucleic acid sequence set forth in SEQ ID NO: 17. In some embodiments, the additional polynucleotide comprises a nucleic acid sequence with at least 83%, at least 85%, at least 87%, at least 95%, or at least 99% homology or identity to SEQ ID NO: 17, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 17. Each possibility represents a separate embodiment of the invention.

In some embodiments, the additional polynucleotide comprises or consists of the nucleic acid sequence set forth in SEQ ID NO: 18. In some embodiments, the additional polynucleotide comprises a nucleic acid sequence with at least 83%, at least 85%, at least 87%, at least 95%, or at least 99% homology or identity to SEQ ID NO: 18, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 18. Each possibility represents a separate embodiment of the invention.

In some embodiments, the additional polynucleotide comprises or consists of the nucleic acid sequence set forth in SEQ ID NO: 19. In some embodiments, the additional polynucleotide comprises a nucleic acid sequence with at least 83%, at least 85%, at least 87%, at least 95%, or at least 99% homology or identity to SEQ ID NO: 19, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 19. Each possibility represents a separate embodiment of the invention.

In some embodiments, the additional polynucleotide comprises or consists of the nucleic acid sequence set forth in SEQ ID NO: 20. In some embodiments, the additional polynucleotide comprises a nucleic acid sequence with at least 83%, at least 85%, at least 87%, at least 95%, or at least 99% homology or identity to SEQ ID NO: 20, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 20. Each possibility represents a separate embodiment of the invention.

In some embodiments, the additional polynucleotide comprises or consists of the nucleic acid sequence set forth in SEQ ID NO: 21. In some embodiments, the additional polynucleotide comprises a nucleic acid sequence with at least 83%, at least 85%, at least 87%, at least 95%, or at least 99% homology or identity to SEQ ID NO: 21, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 21. Each possibility represents a separate embodiment of the invention.

In some embodiments, the additional polynucleotide comprises or consists of the nucleic acid sequence set forth in SEQ ID NO: 22. In some embodiments, the additional polynucleotide comprises a nucleic acid sequence with at least 83%, at least 85%, at least 87%, at least 95%, or at least 99% homology or identity to SEQ ID NO: 22, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 22. Each possibility represents a separate embodiment of the invention.

In some embodiments, the additional polynucleotide comprises or consists of the nucleic acid sequence set forth in SEQ ID NO: 23. In some embodiments, the additional polynucleotide comprises a nucleic acid sequence with at least 83%, at least 85%, at least 87%, at least 95%, or at least 99% homology or identity to SEQ ID NO: 23, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 23. Each possibility represents a separate embodiment of the invention.

In some embodiments, the additional polynucleotide comprises or consists of the nucleic acid sequence set forth in SEQ ID NO: 24. In some embodiments, the additional polynucleotide comprises a nucleic acid sequence with at least 83%, at least 85%, at least 87%, at least 95%, or at least 99% homology or identity to SEQ ID NO: 24, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 24. Each possibility represents a separate embodiment of the invention.

In some embodiments, the additional polynucleotide comprises or consists of the nucleic acid sequence set forth in SEQ ID NO: 25. In some embodiments, the additional polynucleotide comprises a nucleic acid sequence with at least 83%, at least 85%, at least 87%, at least 95%, or at least 99% homology or identity to SEQ ID NO: 25, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 25. Each possibility represents a separate embodiment of the invention.

In some embodiments, the additional polynucleotide comprises or consists of the nucleic acid sequence set forth in SEQ ID NO: 26. In some embodiments, the additional polynucleotide comprises a nucleic acid sequence with at least 83%, at least 85%, at least 87%, at least 95%, or at least 99% homology or identity to SEQ ID NO: 26, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 26. Each possibility represents a separate embodiment of the invention.

In some embodiments, the additional polynucleotide comprises or consists of the nucleic acid sequence set forth in SEQ ID NO: 27. In some embodiments, the additional polynucleotide comprises a nucleic acid sequence with at least 83%, at least 85%, at least 87%, at least 95%, or at least 99% homology or identity to SEQ ID NO: 27, or any value and range therebetween. Each possibility represents a separate embodiment of the invention. In some embodiments, the additional polynucleotide comprises a nucleic acid sequence with 83% to 100%, 88% to 100%, 90% to 100%, or 95% to 100% homology or identity to SEQ ID NO: 27. Each possibility represents a separate embodiment of the invention.

In some embodiments, the additional polynucleotide comprises at least 2, at least 3, at least 5, at least 7, at least 10, or at least 11 different nucleic acid sequences, or any value and range therebetween, wherein each of the different nucleic acid sequences is selected from SEQ ID Nos: 16-27. Each possibility represents a separate embodiment of the invention. In some embodiments, the additional polynucleotide comprises 1-12, 2-12, 4-12, 6-12, 8-12, 10-12, 2-6, 1-3, or 2-3 different nucleic acid sequences, wherein each of the different nucleic acid sequences is selected from SEQ ID Nos: 16-27. In some embodiments, the additional polynucleotide comprises a plurality of additional polynucleotide molecules, wherein each of the plurality of the additional polynucleotide molecules comprises a different nucleic acid sequence, and wherein the different nucleic acid sequences are selected from SEQ ID Nos.: 16-27. In some embodiments, the additional polynucleotide encodes a protein characterized by methyl group transferring activity. In some embodiments, the additional polynucleotide encodes a protein characterized by having an activity of transferring a methyl group to a substrate. In some embodiments, the polynucleotide encodes a protein being a methyltransferase. In some embodiments, the methyltransferase is an O-methyltransferase. In some embodiments, methyltransferase is a methyltransferase derived from a cactus. In some embodiments, the cactus is apeyote. In some embodiments, the additional polynucleotide encoding a methyl transferring protein is selected from SEQ ID Nos: 16-27, or an analog thereof having at least 70%, 80%, 90%, or 95% homology or identity thereto.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing a decarboxylase, hydroxylase, methyl transferase, or any combination thereof, encoding nucleic acid sequence as disclosed herein. In some embodiments, the artificial vector is for use in heterologous expression of a decarboxylase, hydroxylase, methyl transferase, or any combination thereof, encoding nucleic acid sequence as disclosed herein in a cell, a tissue, or an organism. In some embodiments, the artificial vector is for use in producing or the production of mescaline in a cell, a tissue, or an organism.

Expressing a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. [096] The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3: 1671-1680 (1984); and Brogli et al., Science 224:838-843 (1984)] or heat shock promoters, e.g., soybean hspl7.5-E or hspl7.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-lMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or agrobacterium disclosed herein.

In some embodiments, the protein comprises or consists of the amino acid sequence set forth in SEQ ID NO: 28. In some embodiments, the protein comprises an amino acid sequence having at least 87%, at least 92%, at least 95%, or at least 99% homology or identity to SEQ ID NO: 28, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 28. Each possibility represents a separate embodiment of the invention.

In some embodiments, the protein comprises or consists of the amino acid sequence set forth in SEQ ID NO: 29. In some embodiments, the protein comprises an amino acid sequence having at least 87%, at least 92%, at least 95%, or at least 99% homology or identity to SEQ ID NO: 29, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 29. Each possibility represents a separate embodiment of the invention.

In some embodiments, the protein comprises or consists of the amino acid sequence set forth in SEQ ID NO: 30. In some embodiments, the protein comprises an amino acid sequence having at least 87%, at least 92%, at least 95%, or at least 99% homology or identity to SEQ ID NO: 30, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 30. Each possibility represents a separate embodiment of the invention.

In some embodiments, the protein comprising an amino acid sequence selected from SEQ ID Nos: 28-30, or an analog thereof comprising 80-100% homology or identity thereto is characterized by decarboxylating activity, as described herein. In some embodiments, the protein is characterized by having an activity of releasing a carboxyl group from a substrate (such as resulting with the release of CO₂).

In some embodiments, the protein comprises or consists of the amino acid sequence set forth in SEQ ID NO: 31. In some embodiments, the protein comprises an amino acid sequence having at least 87%, at least 92%, at least 95%, or at least 99% homology or identity to SEQ ID NO: 31, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 31. Each possibility represents a separate embodiment of the invention.

In some embodiments, the protein comprises or consists of the amino acid sequence set forth in SEQ ID NO: 32. In some embodiments, the protein comprises an amino acid sequence having at least 87%, at least 92%, at least 95%, or at least 99% homology or identity to SEQ ID NO: 32, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 32. Each possibility represents a separate embodiment of the invention.

In some embodiments, the protein comprises or consists of the amino acid sequence set forth in SEQ ID NO: 33. In some embodiments, the protein comprises an amino acid sequence having at least 87%, at least 92%, at least 95%, or at least 99% homology or identity to SEQ ID NO: 33, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 33. Each possibility represents a separate embodiment of the invention.

In some embodiments, the protein comprises or consists of the amino acid sequence set forth in SEQ ID NO: 34. In some embodiments, the protein comprises an amino acid sequence having at least 87%, at least 92%, at least 95%, or at least 99% homology or identity to SEQ ID NO: 34, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 34. Each possibility represents a separate embodiment of the invention.

In some embodiments, the protein comprises or consists of the amino acid sequence set forth in SEQ ID NO: 35. In some embodiments, the protein comprises an amino acid sequence having at least 87%, at least 92%, at least 95%, or at least 99% homology or identity to SEQ ID NO: 35, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 35. Each possibility represents a separate embodiment of the invention.

In some embodiments, the protein comprises or consists of the amino acid sequence set forth in SEQ ID NO: 36. In some embodiments, the protein comprises an amino acid sequence having at least 87%, at least 92%, at least 95%, or at least 99% homology or identity to SEQ ID NO: 36, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 36. Each possibility represents a separate embodiment of the invention.

In some embodiments, the protein comprises or consists of the amino acid sequence set forth in SEQ ID NO: 37. In some embodiments, the protein comprises an amino acid sequence having at least 87%, at least 92%, at least 95%, or at least 99% homology or identity to SEQ ID NO: 37, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 37. Each possibility represents a separate embodiment of the invention.

In some embodiments, the protein comprises or consists of the amino acid sequence set forth in SEQ ID NO: 38. In some embodiments, the protein comprises an amino acid sequence having at least 87%, at least 92%, at least 95%, or at least 99% homology or identity to SEQ ID NO: 38, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 38. Each possibility represents a separate embodiment of the invention.

In some embodiments, the protein comprises or consists of the amino acid sequence set forth in SEQ ID NO: 39. In some embodiments, the protein comprises an amino acid sequence having at least 87%, at least 92%, at least 95%, or at least 99% homology or identity to SEQ ID NO: 39, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 39. Each possibility represents a separate embodiment of the invention.

In some embodiments, the protein comprises or consists of the amino acid sequence set forth in SEQ ID NO: 40. In some embodiments, the protein comprises an amino acid sequence having at least 87%, at least 92%, at least 95%, or at least 99% homology or identity to SEQ ID NO: 40, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 40. Each possibility represents a separate embodiment of the invention.

In some embodiments, the protein comprises or consists of the amino acid sequence set forth in SEQ ID NO: 41. In some embodiments, the protein comprises an amino acid sequence having at least 87%, at least 92%, at least 95%, or at least 99% homology or identity to SEQ ID NO: 41, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 41. Each possibility represents a separate embodiment of the invention.

In some embodiments, the protein comprises or consists of the amino acid sequence set forth in SEQ ID NO: 42. In some embodiments, the protein comprises an amino acid sequence having at least 87%, at least 92%, at least 95%, or at least 99% homology or identity to SEQ ID NO: 42, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 42. Each possibility represents a separate embodiment of the invention.

In some embodiments, the protein comprises or consists of the amino acid sequence set forth in SEQ ID NO: 60. In some embodiments, the protein comprises an amino acid sequence having at least 87%, at least 92%, at least 95%, or at least 99% homology or identity to SEQ ID NO: 60, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 60. Each possibility represents a separate embodiment of the invention.

In some embodiments, the protein comprises or consists of the amino acid sequence set forth in SEQ ID NO: 61. In some embodiments, the protein comprises an amino acid sequence having at least 87%, at least 92%, at least 95%, or at least 99% homology or identity to SEQ ID NO: 61, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 61. Each possibility represents a separate embodiment of the invention.

In some embodiments, the protein comprises or consists of the amino acid sequence set forth in SEQ ID NO: 62. In some embodiments, the protein comprises an amino acid sequence having at least 87%, at least 92%, at least 95%, or at least 99% homology or identity to SEQ ID NO: 62, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 62. Each possibility represents a separate embodiment of the invention.

In some embodiments, the protein comprises or consists of the amino acid sequence set forth in SEQ ID NO: 63. In some embodiments, the protein comprises an amino acid sequence having at least 87%, at least 92%, at least 95%, or at least 99% homology or identity to SEQ ID NO: 63, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 63. Each possibility represents a separate embodiment of the invention.

In some embodiments, the protein comprising an amino acid sequence selected from SEQ ID Nos: 31-42, and 60-63, or an analog thereof comprising 80-100% homology or identity thereto is characterized by hydroxylating activity, as described herein. In some embodiments, the protein is characterized by having an activity of introducing a hydroxyl group into a substrate.

In some embodiments, the protein comprises or consists of the amino acid sequence set forth in SEQ ID NO: 43. In some embodiments, the protein comprises an amino acid sequence having at least 87%, at least 92%, at least 95%, or at least 99% homology or identity to SEQ ID NO: 43, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 43. Each possibility represents a separate embodiment of the invention.

In some embodiments, the protein comprises or consists of the amino acid sequence set forth in SEQ ID NO: 44. In some embodiments, the protein comprises an amino acid sequence having at least 87%, at least 92%, at least 95%, or at least 99% homology or identity to SEQ ID NO: 44, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 44. Each possibility represents a separate embodiment of the invention.

In some embodiments, the protein comprises or consists of the amino acid sequence set forth in SEQ ID NO: 45. In some embodiments, the protein comprises an amino acid sequence having at least 87%, at least 92%, at least 95%, or at least 99% homology or identity to SEQ ID NO: 45, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 45. Each possibility represents a separate embodiment of the invention.

In some embodiments, the protein comprises or consists of the amino acid sequence SEQ ID NO: 46. In some embodiments, the protein comprises an amino acid sequence having at least 87%, at least 92%, at least 95%, or at least 99% homology or identity to SEQ ID NO: 46, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 46. Each possibility represents a separate embodiment of the invention.

In some embodiments, the protein comprises or consists of the amino acid sequence SEQ ID NO: 47. In some embodiments, the protein comprises an amino acid sequence having at least 87%, at least 92%, at least 95%, or at least 99% homology or identity to SEQ ID NO: 47, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 47. Each possibility represents a separate embodiment of the invention.

In some embodiments, the protein comprises or consists of the amino acid sequence SEQ ID NO: 48. In some embodiments, the protein comprises an amino acid sequence having at least 87%, at least 92%, at least 95%, or at least 99% homology or identity to SEQ ID NO: 48, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 48. Each possibility represents a separate embodiment of the invention.

In some embodiments, the protein comprises or consists of the amino acid sequence SEQ ID NO: 49. In some embodiments, the protein comprises an amino acid sequence having at least 87%, at least 92%, at least 95%, or at least 99% homology or identity to SEQ ID NO: 49, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 49. Each possibility represents a separate embodiment of the invention.

In some embodiments, the protein comprises or consists of the amino acid sequence SEQ ID NO: 50. In some embodiments, the protein comprises an amino acid sequence having at least 87%, at least 92%, at least 95%, or at least 99% homology or identity to SEQ ID NO: 50, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 50. Each possibility represents a separate embodiment of the invention.

In some embodiments, the protein comprises or consists of the amino acid sequence SEQ ID NO: 51. In some embodiments, the protein comprises an amino acid sequence having at least 87%, at least 92%, at least 95%, or at least 99% homology or identity to SEQ ID NO: 51, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 51. Each possibility represents a separate embodiment of the invention.

In some embodiments, the protein comprises or consists of the amino acid sequence SEQ ID NO: 52. In some embodiments, the protein comprises an amino acid sequence having at least 87%, at least 92%, at least 95%, or at least 99% homology or identity to SEQ ID NO: 52, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 52. Each possibility represents a separate embodiment of the invention.

In some embodiments, the protein comprises or consists of the amino acid sequence set forth in SEQ ID NO: 53. In some embodiments, the protein comprises an amino acid sequence having at least 87%, at least 92%, at least 95%, or at least 99% homology or identity to SEQ ID NO: 53, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 53. Each possibility represents a separate embodiment of the invention.

In some embodiments, the protein comprises or consists of the amino acid sequence set forth in SEQ ID NO: 54. In some embodiments, the protein comprises an amino acid sequence having at least 87%, at least 92%, at least 95%, or at least 99% homology or identity to SEQ ID NO: 54, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 54. Each possibility represents a separate embodiment of the invention.

In some embodiments, the protein comprising an amino acid sequence selected from SEQ ID Nos: 43-54, or an analog thereof comprising 80-100% homology or identity thereto is characterized by methyl group transferring activity, as described herein. In some embodiments, the protein is characterized by having an activity of transferring a methyl group to a substrate.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides, and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

According to some embodiments, there is provided a transgenic cell comprising: (a) the polynucleotide disclosed herein; (b) the artificial nucleic acid molecule disclosed herein; (c) the plasmid or agrobacterium disclosed herein; (d) the protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture. In some embodiments, a unicellular organism comprises a fungus or a bacterium. In some embodiments, the fungus is a yeast cell. In some embodiments, the cell is a prokaryote or a eukaryote cell. In some embodiments, the yeast cell is or comprises a Saccharomyces cerevisiae cell.

In some embodiments, the transgenic cell comprises at least partially inactive acetyl transferring enzyme, e.g., acetyltransferase. In some embodiments, the transgenic cell comprises fully inactive acetyl transferring enzyme, e.g., acetyltransferase. In some embodiments, the transgenic cell is devoid of an acetyl transferring enzyme, e.g., acetyltransferase. In some embodiments, the transgenic cell comprises a knocked-out acetyl transferring enzyme encoding gene or a transcript thereof. In some embodiments, the transgenic cell comprises a knocked-down (or silenced) acetyl transferring enzyme encoding gene or a transcript thereof. In some embodiments, the transgenic cell comprises RNA interfering (RNAi) oligonucleotides capable of hybridizing to, inducing degradation of, or both, an acetyl transferring enzyme encoding gene or a transcript thereof. In some embodiments, the transgenic cell synthesizes RNAi oligonucleotides hybridizing to, inducing degradation of, or both, an acetyl transferring enzyme encoding gene or a transcript thereof. In some embodiments, the transgenic cell further comprises RNAi oligonucleotides capable of hybridizing to, inducing degradation of, or both, an acetyl transferring enzyme encoding gene or a transcript thereof. In some embodiments, the acetyl transferring enzyme comprises or is an arylalkyl amine N-acetyltransferase (AANAT). In some embodiments, the cell is a Saccharomyces cerevisiae cell. In some embodiments, the acetyl transferring enzyme comprises or is arylalkyl amine N-acetyltransferase from S. cerevisiae (scAANAT, GenBank accession number S49826).

According to some embodiments, there is provided an extract derived from a transgenic cell disclosed herein, or any fraction thereof. In some embodiments, the extract comprises the polynucleotide of the invention, an isolated DNA molecule as disclosed herein, an isolated protein as disclosed herein, or any combination thereof.

According to some embodiments, there is provided a homogenate, lysate, extract, derived from a transgenic cell disclosed herein, any combination thereof, or any fraction thereof. In some embodiments, the extract comprises mescaline or a precursor thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue, or organ thereof, comprising at least one transgenic plant cell of the invention. In some embodiments, the transgenic plant, transgenic plant tissue or plant part, comprises: (a) the polynucleotide disclosed herein; (b) the plasmid or agrobacterium disclosed herein; (c) the isolated protein of the invention; (d) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant further comprises at least one gene encoding a deficient, inactive, non-functional, or any combination thereof, an N-methyltransferase gene. In some embodiments, at least one N-methyltransferase gene is knocked out, knocked down, silenced, or any combination thereof, in the transgenic plant. In some embodiments, at least one N-methyltransferase gene of the transgenic plant comprises a premature stop codon, e.g., a nonsense mutation.

In some embodiments, a wild type and/or non-mutated N-methyltransferase gene comprises the nucleic acid sequence set forth in SEQ ID NO: 55.

In some embodiments, the transgenic plant comprises a nucleic acid sequence comprising SEQ ID NO: 55, comprising one or more mutations rendering a protein encoded by same as inactive, partially active, non-functional, or any combination thereof. In some embodiments, the one or more mutation comprises a nonsense mutation, a missense mutation, a deletion, an inversion, a truncation, or any combination thereof. In some embodiments, the transgenic plant is devoid of a nucleic acid sequence comprising SEQ ID NO: 55 or a protein encoded by same. In some embodiments, a genome of the transgenic plant is devoid of a nucleic acid sequence comprising SEQ ID NO: 55.

According to some embodiments, there is provided a composition comprising: (a) polynucleotide of the invention (for example, an isolated DNA molecule); (b) plasmid or agrobacterium; (c) isolated protein of the invention; (d) transgenic cell; (e) extract; (f) transgenic plant tissue or plant part; or (g) any combination of (a) to (f), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to another aspect, there is provided a method for synthesizing mescaline.

In some embodiments, the method comprises the steps: (a) providing a cell comprising: (i) an isolated DNA molecule comprising a nucleic acid sequence set forth in SEQ ID Nos: 1-15, and 56-59, or any combination thereof; (ii) a plasmid or an agrobacterium comprising a nucleic acid sequence set forth in SEQ ID Nos: 1-15, and 56-59, or any combination thereof; or (iii) both (i) and (ii); and (b) culturing the cell from step (a) such that at least one polypeptide encoded by the isolated DNA molecule, the plasmid or agrobacterium, or both, is expressed.

In some embodiments, the isolated DNA molecule, the plasmid, the agrobacterium, or any combination thereof, comprises SEQ ID Nos: 1, 5, 6, 15, or any combination thereof. In some embodiments, the isolated DNA molecule, the plasmid, the agrobacterium, or any combination thereof, comprises SEQ ID Nos: 1, 5, 6, and 15. In some embodiments, the isolated DNA molecule, the plasmid, the agrobacterium, or any combination thereof, further comprises a nucleic acid sequence set forth in SEQ ID Nos: 16-27, or any combination thereof. In some embodiments, the isolated DNA molecule, the plasmid, the agrobacterium, or any combination thereof, further comprises SEQ ID Nos: 17, 21, 26, 27, or any combination thereof. In some embodiments, the isolated DNA molecule, the plasmid, the agrobacterium, or any combination thereof, further comprises SEQ ID Nos: 17, 21, 26, and 27. In some embodiments, the isolated DNA molecule, the plasmid, the agrobacterium, or any combination thereof, comprises SEQ ID Nos: 1, 5, 6, 15, 17, 21, 26, and 27.

In some embodiments, the at least one polypeptide is characterized by having an activity selected from: releasing a carboxyl group from a substrate, introducing a hydroxyl group into a substrate, transferring a methyl group from a donor molecule to a substrate, or any combination thereof. In some embodiments, the substrate comprises a substrate molecule. In some embodiments, the substrate comprises an organic compound or molecule. In some embodiments, culturing comprises supplementing the cell with an effective amount of a mescaline precursor.

In some embodiments, a mescaline precursor comprises any compound known to a person of skill in the art to be involved and/or being a member of the mescaline biosynthetic pathway.

In some embodiments, a mescaline precursor is selected from: L-tyrosine, L-DOPA, Dopamine, 3-MeO-tyramine, 3,4-diOH-5-MeO-phenethylamine, 4-OH-3,5-diMeO-phenethylamine, or any combination thereof.

In some embodiments, the cell comprises or is a transgenic cell or a cell transfected with the isolated DNA molecule of the invention or the plasmid or agrobacterium of the invention.

In some embodiments, the method further comprises a step before step (a), comprising introducing or transfecting the cell with the plasmid or agrobacterium.

Method for introducing or transfecting a cell with an artificial nucleic acid molecule or vector (e.g., plasmid and/or agrobacterium) are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

In some embodiments, artificial nucleic acid molecule or vector comprises: a plasmid, an agrobacterium, or both.

As used herein, the terms “transfecting” and “introducing” are interchangeable.

In some embodiments, the method comprises contacting a mescaline precursor with an effective amount of at least one isolated polypeptide comprising an amino acid sequence selected from SEQ ID Nos: 28-42, and 60-63, or any combination thereof. In some embodiments, the contacting is with isolated proteins. In some embodiments, each isolated protein of the different isolated proteins comprises a different amino acid. In some embodiments, the different isolated protein comprises the amino acid sequences set forth in SEQ ID Nos: 28, 32, 33, 42, or any combination thereof. In some embodiments, the different isolated protein comprises the amino acid sequences set forth in SEQ ID Nos: 28, 32, 33, and 42. In some embodiments, the method further comprises contacting the mescaline precursor with an isolated polypeptide or a plurality thereof, comprising an amino acid sequence having at least 75%, 80%, 90%, 95%, or 99% homology or identity to SEQ ID Nos.: 43-54, or any value and range therebetween. Each possibility represents a separate embodiment of the invention. In some embodiments, the method further comprises contacting the mescaline precursor with an isolated polypeptide or a plurality thereof, comprising an amino acid sequence having at least 75%, 80%, 90%, 95%, or 99% homology or identity to SEQ ID Nos.: 44, 48, 53, 54, or any value and range therebetween. Each possibility represents a separate embodiment of the invention. In some embodiments, the method further comprises contacting the mescaline precursor with an isolated polypeptide or a plurality thereof, comprising an amino acid sequence having at least 75%, 80%, 90%, 95%, or 99% homology or identity to SEQ ID Nos.: 44, 48, 53, and 54. Each possibility represents a separate embodiment of the invention.

In some embodiments, contacting is in a cell-free system.

According to another aspect, there is provided a mescaline synthesized according to the method of the invention.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell is the transgenic cell disclosed herein.

In some embodiments, the transgenic cell or the transfected cell comprises an isolated DNA molecule comprising a nucleic acid sequence set forth in SEQ ID Nos: 1-27, and 56-59, or any combination thereof. In some embodiments, the transgenic cell or the transfected cell comprises an isolated DNA molecule comprising a nucleic acid sequence set forth in SEQ ID Nos: 1, 5, 6, 15, 17, 21, 26, 27, or any combination thereof.

In some embodiments, the transgenic cell or the transfected cell comprises the polynucleotide of the invention or a plurality thereof, as disclosed herein. In some embodiments, the transgenic cell or the transfected cell comprises the plasmid or agrobacterium as disclosed herein. In some embodiments, the cell is a transgenic cell, or a cell transfected with a polynucleotide as disclosed herein.

In some embodiments, the method further comprises a step proceeding or after step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transgenic cell or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof, or (c) any combination of (a) and (b), and an acceptable carrier, as described herein.

In some embodiments, a portion comprises a fraction or a plurality thereof.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Materials

Unless otherwise stated, all the analytical metabolites were ≥95% pure. Mescaline (hydrochloride) 1, dopamine (hydrochloride) 3, 3-MeO-tyramine (hydrochloride) 7 and hordenine 13 were purchased from Cayman Chemical (Ann Arbor, MI, USA). L-DOPA 5 and L-tyrosine 2 were purchased from Sigma-Aldrich (Rehovot, Israel), 3,4-diOH-5-MeO-phenethylamine (hydrochloride) 6 was purchased from Rare Chemicals GmbH (Kiel, Germany), 4-OH-3,5-diMeO-phenethylamine (hydrochloride) 9 was purchased from Ambinter (Orleans, France), and tyramine 4 was purchased from Holland Moran (Yehud, Israel). N-p-trans-Coumaroyltyramine, N-trans-caffeoyltyramine, N-trans-feruloyltyramine and N-trans-feruloyl-3-methoxytyramine were purchased from BioBioPha (Yunnan, China). All cacti were acquired from a local nursery. Validation of assignment of Peyote and L. diffusa to the appropriate species was performed on the basis of chemical profiling.

### LC-MS Chemical Analysis

Unless otherwise stated, 100 mg frozen powdered plant tissue were extracted with 300 μl 80% methanol and 0.1% formic acid, sonicated for 15 min, agitated for 30 min, and centrifuged at 14,000 g for 10 min. The supernatant was filtered through a 0.22 μm syringe filter. Samples were analyzed using an ultrahigh-performance liquid chromatography (UPLC, Waters Acquity) with a diode array detector connected either to a quadrupole time-of-flight (XEVO G2-S or Synapt HDMS qTOF systems, Waters), an Orbitrap IQ-X Tribrid MS, or an Obitrap Exploris 240 MS (Thermo Scientific, Bremen, Germany). The chromatographic separation was performed on a 100 mm×2.1 mm i.d. (internal diameter), 1.7 μm UPLC BEH-T3 C18 column (Waters Acquity). The mobile phase consisted of 0.1% formic acid in acetonitrile:water (1:99, v/v; phase A) and 0.1% formic acid in acetonitrile (phase B). The multistep method was as follows: Initial conditions were 0% B for 4 min, raised to 75% B until 12 min, raised to 100% B in 0.2 min, held at 100% B until 15 min, decreased to 0% B in 0.2 min, and held at 0% B until 18 min for re-equilibration of the system. The flow rate was 0.3 ml min⁻¹, and the column temperature was kept at 35° C.

Electrospray ionization (ESI) was used in positive ionization mode at an m/z range of 50-1,000 Da. With the qTOF system masses were detected with the following settings: capillary 1 kV, source temperature 140° C., desolvation temperature 450° C., and desolvation gas flow 800 l h⁻¹. MS/MS experiments were performed according to the specific protonated masses with following settings: capillary spray of 1 kV; cone voltage of 30 eV; the collision energy ramp was 10-45 eV. Argon was used as the collision gas. On the Orbitrap MS systems the source parameters were: sheath gas flow rate, auxiliary gas flow rate and sweep gas flow rate: 45, 10 and 1 arbitrary units, respectively; vaporizer temperature: 275° C.; ion transfer tube temperature: 275° C.; spray voltage: 2.3 kV. The instrument was operated in full MS¹ with data dependent MS/MS (MS-dd-MS²). Data acquisition in full MS¹ mode was 60,000 resolution, normalized automatic gain control target of 25% and a maximum injection time of 50 ms. Data acquisition in dd-MS² mode was with 15,000 resolution, a normalized AGC target of 20%, maximum IT of 150 ms, isolation window of 1.5 m/z and normalized collision energy of 40. Identification of metabolites 1-7 and 9 was performed according to analytical standards. All the other metabolites were putatively identified according to LC-MS/MS as detailed in FIGS. 11-13. Identification of some metabolites was further validated in the in vitro and/or in planta assays.

### Absolute Quantification of Mescaline 1

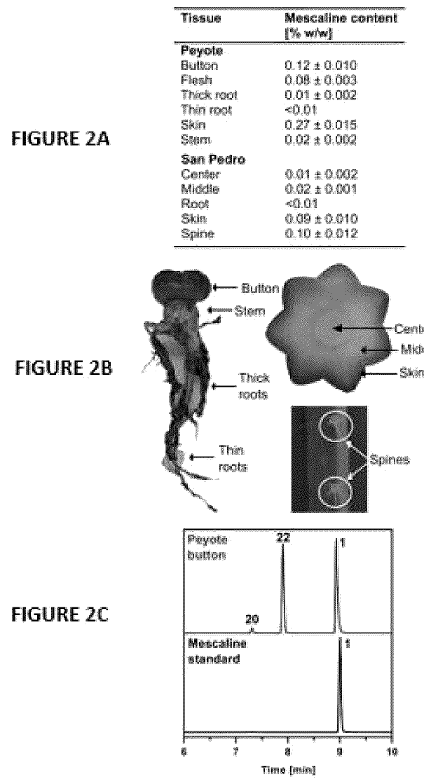

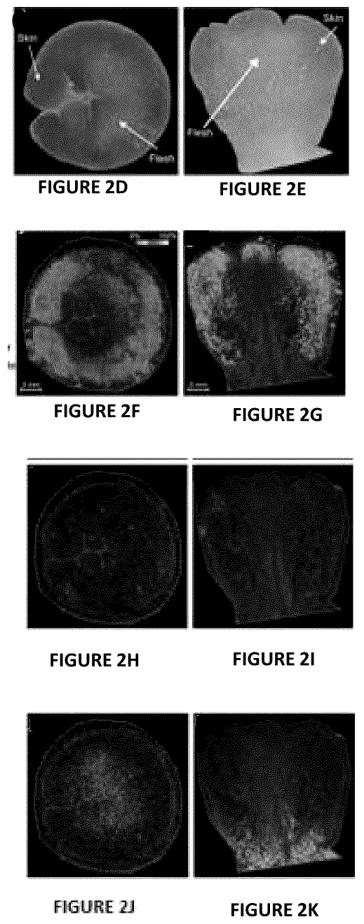

Fresh samples of Peyote whole button, stem, thick and thin roots were collected from medium-size cacti (FIG. 2B). The green sub-epidermal and flesh tissues of buttons were separated using a scalpel and analyzed separately. San Pedro flesh tissues were dissected with a scalpel to skin (green sub-epidermal area), middle and center tissues. Spines correspond to the flesh tissue surrounding the spines (FIG. 2B). For the extraction, 100 mg of frozen powders were accurately weighed in triplicates, extracted with 1 ml 80% methanol and 0.1% formic acid, and prepared as previously described. Samples were injected in several dilutions to fit into the linear range of the calibration curve. Injections were performed on the IQ-X Tribrid MS system in full MS¹ mode as previously described.

### MALDI Imaging

For localization of metabolites to Peyote tissues, two young fresh cacti were cryo-sectioned horizontally and laterally (n=2 biological replicates each) and matrix was sprayed using a TM sprayer (HTX Technologies, USA) with 2,5-dihydroxybenzoic acid (DHB; 40 mg ml⁻¹ in 70% containing 0.2% trifluoroacetic acid). The nozzle temperature was set at 70° C. and the DHB matrix solution sprayed 16 passes over the tissue sections at a linear velocity of 120 cm min⁻¹ with a flow rate of 50 μl min⁻¹. Sections were imaged with a Nikon DS-Ri2 microscope. MALDI imaging was performed using a 7 T Solarix FT-ICR (Fourier Transform Ion Cyclotron Resonance) mass spectrometer (Bruker Daltonics). Data acquisition and analysis were performed using flexImaging V4.1 (Bruker). The datasets were collected in positive ionization using lock mass calibration (DHB matrix peak: [3DHB+H-3H₂O]⁺, m/z 409.055408 Da) at a frequency of 1 kHz and a laser power of 40%, with 200 laser shots per pixel and 50 μm pixel size. Each mass spectrum was recorded in the range of m/z 120-2,000 in broadband mode with a Time Domain for Acquisition of 1M, providing an estimated resolving power of 115,000 at m/z 400. The spectra were normalized to root-mean-square intensity and MALDI images were plotted at theoretical m/z±0.005% with pixel interpolation on.

### Genome Assembly and RNA Sequencing and Annotation

High molecular weight DNA was extracted from young frozen green tissue and shipped for sequencing in Edinburgh Genomics. Sequencing was done in a Pacbio Sequel II platform with ~8-kilobase DNA SMRT bell library preparation. Two different SMRT 8M cells were used, yielding a total of 35.5 Gb of HiFi data. Hifiasm software (Cheng et al., 2021; Nat. Methods) was used to assemble the genome to produce 11×3.3 Gb genome assembly.

RNA was extracted from six tissues of Peyote, L. difussa, and San Pedro. RNA integrity was checked using a TapeStation instrument. Paired-end Illumina libraries were prepared and sequenced on Illumina HiSeq 3000 instrument (PE 2×150, ~40 M reads per sample) and processed following Freedman and Weeks (informatics.fas.harvard.edu/best-practices-for-de-novo-transcriptome-assembly-with-trinity.html) guidelines. Briefly, random sequencing errors were corrected using Rcorrector (Song and Florea, 2015; Gigascience) and uncorrectable reads were removed. Adaptor and quality trimming were performed using TrimGalore!(github.com/FelixKrueger/TrimGalore). Ribosomal RNA was filtered by discarding reads mapping to SILVA_132_LSURef and SILVA_138_SSURef non-redundant databases using bowtie2 (Langmead and Salzberg, 2012; Nat. Methods). Fastq quality checks on each of the steps were performed using MultiQC (Ewels et al., 2016; Bioinformatics). The remaining reads were pooled and used for genome-independent de novo transcriptome assembly using Trinity (Grabherr et al., 2011; Nat. Biotechnol.).

The Iso-Seq data was obtained from a pool of six tissues of L. williamsii and processed with isoseq3 (github.com/PacificBiosciences/IsoSeq). Fused and unspliced transcripts were removed, and only polyA-positive transcripts were kept for a unique set of high-quality isoforms. Iso-Seq and Trinity transcripts were aligned to the assembly using minimap2 (Li, 2018; Bioinformatics) and the BAM files were used to produce RNA-based gene models using isoseq collapse (github.com/PacificBiosciences/IsoSeq). Gene functional annotation was performed for the predicted mature transcripts using TransDecoder (github.com/TransDecoder/TransDecoder/), which takes into account HIMER (Finn et al., 2011; Nucleic Acids Res) hits against PFAM (Finn et al., 2014; Nucleic Acids Res) and BLASTP (Altschul et al., 1990; J. Mol. Biol) hits against UniProt (UniProt Consortium, 2008; Nucleic Acids Res.) databases for similarity retention criteria. Further annotation of protein-coding transcripts was performed by taking the best hit of BLASTP searches against other plant protein databases (Uniprot protein FASTA files of arabidopsis id UP000006548_3702, tomato id UP000004994_4081, rice id UP000059680_39947, sugar beet id Bvulgaris_548_EL10_1.0 and spinach id UP000054095_3562). Signal peptides were predicted with SignalP (Almagro et al., 2019; Nat. Biotech.), transmembrane domains were predicted with TMH1MI (Krogh et al., 2001; J. Mol. Biol.), and GO and KEGG terms were obtained with Trinotate (Bryant et al., 2017; Cell Rep.). BUSCO (Simao et al., 2015; Bioinformatics) was used at multiple stages of the analysis to assess the completeness of the different versions of both the transcriptome and the genome. Circos and gene cluster plots were generated from the gff file. The circos plot was made with the R circlize (Gu et al., 2014; Bioinformatics) package and the gene clusters plots were made with the gggenes (github.com/wilkox/gggenes) package. The full script used for the functional annotation of the proteins can be found in https://github.com/Luisitox/Cacti_paper.

### Phylogenetic Analyses Offunctionally Tested Enzymes

The selection of the proteins for each of the families analyzed in this study was based on functionally tested enzymes according to studies referenced in each Figure. The Maximum Likelihood trees were constructed with 300 bootstrap tests based on a MUSCLE multiple alignment using the MEGA11 software. The evolutionary distances were computed using the JTTmatrix-based method. The phylogenetic relationship of the cactoideae family was created with BioRender.com according to a synthesis of existing literature (Hernindez-Hernindez et al., 2011; Am. J. Bot.) and represents a preliminary interpretation of the relationships among the taxa in this study.

### Orthology Analyses

Proteomes of the three cacti were obtained from each genome/transcriptome annotation. In the case of Trinity assemblies, CD-HIT (Fu et al., 2012; Bioinformatics) was run to cluster closely related transcripts with a tolerance of 90%. For the Pacbio-Based assembly, the longest functional protein was used. Orthogroups and their phylogenetic relationship were inferred with Orthofinder (Emms et al., 2019; Genome Biol.). Proteins belonging to the same orthogroups as LwTyDC2, LwOMT1, LwOMT5, LwOMT10, LwOMT11, LwNMT1 and LwCYP2 were identified, realigned and their phylogenetic relationship was inferred with MEGA11 as previously described.

Recombinant Expression in E. coli

TyDC2,4,5 and OMT1-11 coding sequences from Peyote were individually cloned into the pET28b vector digested with EcoRI using the ClonExpress II one step cloning kit (Vazyme, Germany). All constructs along with the empty vector as the control were expressed in E. coli BL21(DE3)pLysS competent cells. Bacterial starters were grown overnight in LB medium at 37° C., diluted in fresh LB 1:100, and re-incubated at 37° C. When cultures reached A600=0.6, protein expression was induced with 500 μM of isopropyl-1-thio-o-d-galactopyranoside (IPTG) overnight at 15° C. Bacterial cells were lysed by sonication in 50 mM Tris-HCl pH 8.0, 0.5 mM phenylmethylsulfonyl fluoride (PMSF, Sigma Aldrich) solution in isopropanol, 10% glycerol and protease inhibitor cocktail (Sigma Aldrich), and 1 mg ml⁻¹ lysozyme (Sigma Aldrich). The whole-cell extract was kept for functional activity.

### TyDC and OMT Enzyme Assays

Individual TyDC assays were carried out as described by Lehmann and Pollmann (Lehmann and Pollmann, 2009; FEBS Lett.) with some modifications. Enzyme assays were performed in 50 μL with 50 mM sodium phosphate buffer at pH 7.5, 1 mM PLP, 2 mM L-tyrosine 2 or L-DOPA 5 and 20 μl of the lysate enzyme solution. Individual OMT enzyme assays were performed in 50 μL with 50 mM potassium phosphate buffer at pH 7.5, 1 mM SAM, 25 mM ascorbic acid, 20 μl of the lysate enzyme solution, and 1 mM of either dopamine 3, 3,4-diOH-5-MeO-phenethylamine 6, or 4-OH-3,5-diMeO-phenethylamine 9. The assay mixtures were incubated at 30° C. for 2 h, after which the reactions were terminated by the addition of 100 μL ice-cold methanol with 0.1% formic acid, vortexing and centrifugation at 15 000 g for 10 min. The supernatant was filtered and analyzed by LC-MS. Products in each assay were identified according to analytical standards except for 3-OH-4,5-diMeO-phenethylamine 10 and 4-MeO-tyramine 11 which were compared with similar peaks in a Peyote skin extract.

Transient Expression of Selected Genes in N. benthamiana

TyDC2, CYP1-12, PPO1, MT1-4, OMT1,5,10,11 and the previously characterized BvCYP76AD6 were amplified and cloned in pAlpha2-NPT II-Ubq10-CCD-Ter10 vector digested with BsaI using ClonExpress II One Step Cloning kit (Vazyme). For BvCYP76AD6 the inventors used stocks of previously transformed bacteria containing the specific gene (Polturak et al., 2016; New Phyol.). All plasmids were sequenced and transformed into Agrobacterium tumefaciens strain GV3101 by electroporation. A. tumefaciens harboring the overexpression constructs were grown overnight at 28° C. in Luria-Bertani (LB) medium in the presence of kanamycin and gentamycin. Bacterial cells were collected by centrifugation, washed, and resuspended in infiltration buffer (10 mM MES, 2 mM MgCl2, 2 mM Na3PO4, 0.5% glucose and 100 mM acetosyringone) to OD600=0.8. Individual and/or equal volumes of A. tumefaciens suspension with different expression vectors were combined to obtain the desired gene combinations and incubated for 2 h at room temperature. The solutions were infiltrated into 4- or 5-week-old N. benthamiana leaves from the abaxial side using a 1-ml needleless syringe. Unless otherwise stated, substrates (50 μg ml⁻¹ each) were infiltrated into the same leaf areas 2 days after initial infiltration, and all leaves were collected for metabolite analysis after 60-72 h after the substrate infiltration (samples without supplemented substrate were collected at similar time points). To test the activity of LwCYP2 and CYP3, leaves were infiltrated also with 100 μM of 3-MeO-tyramine 7. In the individual experiments with only CYP7 samples were collected 2 and three days after infiltration. Leaf samples were flash frozen and extracted as previously described with 300 μl ice-cold methanol with 0.1% formic acid and analyzed via LC-MS. Identification of metabolites was performed using analytical standards and/or compared with similar peaks in a Peyote skin extract. Analysis of HCAAs was with similar conditions, column and mobile phase as previously described with the following multistep method: Initial conditions were 5% B raised to 33% B until 22 min, raised to 100% B in 0.5 min, held at 100% B until 26 min, decreased to 5% B in 0.5 min, and held at 5% B until 30 min for re-equilibration of the system.

### Molecular Modeling and Docking of LwOMT1, LwOMT11 and LwNMT1

All the molecular modeling procedures were performed using Schrödinger Suite 2022-2 suite of programs (Schrödinger Release 2023-2; Schrödinger, LLC, New York, NY, 2023). The protein structures were prepared employing Protein Preparation Wizard (Sastry et al., 2013; J. Comput. Aid. Mol. Des.). Hydrogens were added, bond orders were assigned, and missing side chains were built using Prime (Jacobson et al., 2022; J. Mol. Biol.). Histidine side chains were left neutral, aspartates and glutamate side chains deprotonated, and lysine and arginine were in cationic form. Finally, the protein structures as well as the final complexes underwent restrained minimization (RMSD=0.5 Å) using OPLS4 forcefield (Lu et al., 2021; J. Chem. Theory Comput.). Structural models for all three enzymes (LwNMT1, LwOMT1 and LwOMT11) were built de novo using AlphaFold 2 software (Jumper et al., 2021; Nature). Each prediction run resulted in five structures and the model with the highest confidence score (pLDDT) was chosen for modeling as follows: 2AN4-1, human phenylethylamine NMT, 6I6K (Papaver somnferum OMT) and 1FP2 (isoflavone OMT from Medicago sativa). For placing the non-methylated substrate and SAM structures into the obtained enzyme 3D models, backbone alignment of known enzyme PDB structures with corresponding regioselectivity to N-methylation, 3-O-hydroxyphenyl and 4-O-hydroxyphenyl was performed. When necessary, manual ligand structure editing in the workspace was carried out. In the resulting complexes, steric clashes of the side chains with SAM or substrates and products were resolved by side chain local minimizations. QM/MM calculations were performed using the QSite utility (Murphy et al., 2000; J. Comp. Chem.). The QM region included the substrate anion, the magnesium cation, a water molecule and SAM as well as side chains of amino acid residues either coordinating the magnesium ion or limiting steric freedom of ligands. For LwOMT1 the amino acid residues H268, N130, F162, L135, D269, M319, N323, L126, F175 and the whole W265 residue; for LwOMT11: N154, F159, F186, F199, Q206, H292, D293, S347 and the whole C289 residue were chosen. The quantum mechanical (QM) region atoms underwent staged geometry optimization ultimately using B3LYP DFT functional and 6+31G(d,p) basis set. The rest of the system was treated on the molecular mechanical (MM) level using OPLS2005 potential. For MM minimization, truncated Newton algorithm was employed with long range forces cutoff equal to 10 Å and the long-range forces were updated every 10 steps.

### Data Availability

All NGS raw sequencing data as well as the primary genome assembly version Lwill_v1 can be found in ENA under the accession number PRJEBXXX. The sequences of the active genes will be deposited in NCBI GenBank. The protein databases used in this study are Uniprot Swiss-Prot release-2022_02, arabidopsis UP000006548_3702, tomato UP000004994_4081, rice UP000059680_39947, sugar beet Bvulgaris_548_EL10_1.0, and spinach UP000054095_3562. The Pfam database was Pfam-A.hmm release 34.0.

### Heterologous Expression in Yeast

CYP76AD131, TyDC2, CYP84A220, OMT1 and OMT11 coding sequences were individually cloned into pESC plasmids allowing simultaneous expression of up to two genes in one plasmid. LwCYP76AD131 was cloned into pESC-Trp; LwCYP84A22O and LwOMTH1 were cloned into pESC-Leu; and LwTyDC2 and LwOMT1 were cloned into pESC-His. The cloning was performed by in-fusion and the pESC vectors were linearized by PCR. The pESC constructs were sequenced and transformed into S. cerevisiae WAT21 using the Yeastmaker yeast transformation system (Clontech) as follows: (a) LwTyDC2-pESC-His and LwCYP76AD131-pESC-Trp; (b) LwCYP76AD131-pESC-Trp; (c) LwCYP84A220-pESC-Leu; and (d) LwTyDC2-LwOMT1-pESC-His, LwCYP76AD131-pESC-Trp and LwOMT1-pESC-Leu. Each strain was additionally transformed with the appropriate EVs as control. Transformed yeast were grown on SD minimal media supplemented with appropriate amino acids and 2% glucose. Colonies were screened and the presence of the transgene was confirmed by colony PCR. For induction of gene expression, transformed cells were grown in 5 mL SD minimal medium with 2% glucose and after 24 h transferred to an SD minimal medium with 2% galactose without additional supplementation or containing 1 mM of either tyramine 4, dopamine 3, 3-MeO-tyramine 7, 3,4-diOH-5-MeO-phenethylamine 6 or 4-OH-3,5-diMeO-phenethylamine 9, and grown for additional 48 h at 30° C.

Cultures were centrifuged at 8,000 g for 1 min and the pellet and supernatant were collected separately. The supernatant was lyophilized, 1 mL of methanol with 0.1% formic acid was added, sonicated for 30 min, centrifuged at 4,300 r.p.m for 10 min and then the clear supernatant was collected and dried under a stream of N₂. Dry residues were dissolved in 200 μL of methanol, sonicated for 20 min, centrifuged at 15,000 g for 10 min and filtered through a 0.22 μm filter for LC-MS analysis. The cell pellet was weighed, double the amount of glass beads (diameter 500 μm) and 500 μL of methanol with 0.1% formic acid was added and vortexed at maximum speed for 10 min. Lysed cells were centrifuged at 21,000 g for 5 min and clear supernatant was collected and dried under a stream of N₂. Dry residues were dissolved in 100 μL of methanol, sonicated for 20 min, centrifuged at 15,000 g for 10 min and filtered through a 0.22 μm filter for LC-MS analysis. Samples were analyzed using the acquired dd-MS² data according to the following transitions: L-tyrosine 2: 182.081→165.055; dopamine 3: 154.086→137.056; tyramine 4: 138.091→121.065; 3,4-diOH-5-MeO-phenethylamine 6: 184.097→167.070; 3-MeO-tyramine 7: 168.102→151.075; 4-OH-3,5-diMeO-phenethylamine 9 and 3-OH-4,5-diMeO-phenethylamine 10: 198.113→181.086; mescaline 1: 212.128→195.101; N-acetyl-tyramine 32: 180.102→121.065; N-acetyl-dopamine 33: 196.097→137.056; N-acetyl-3-MeO-tyramine 34: 210.113→151.075; N-acetyl-3,4-diOH-5-MeO-phenethylamine 35: 226.107→167.070; N-acetyl-4-OH-3,5-diMeO-phenethylamine 36 and N-acetyl-3-OH-4,5-diMeO-phenethylamine 37: 240.123→181.086; N-acetyl-mescaline 1: 254.139→195.101.

### Example 1

### Taxonomically Distant Cacti Produce Mescaline

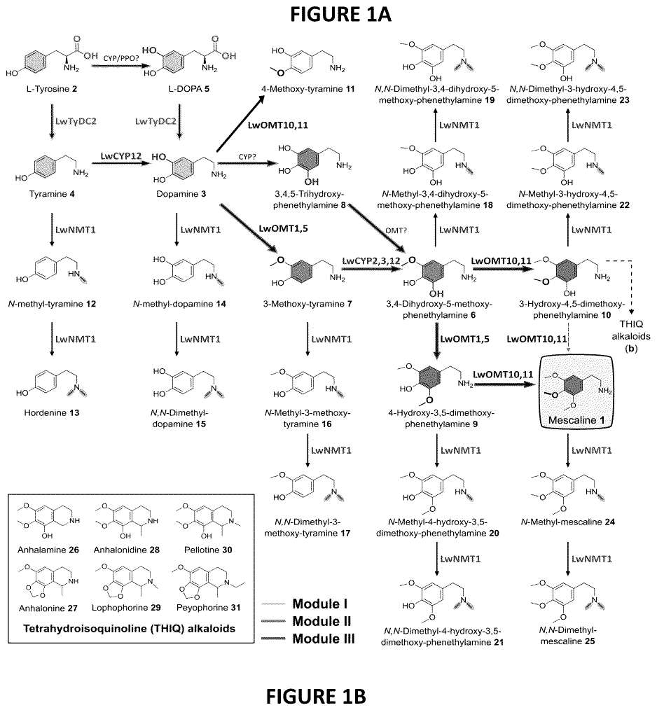

For clarity purposes, the predicted biosynthetic pathway of mescaline 1 in Peyote can be divided into three modules (FIG. 1A): First, the amino acid L-tyrosine 2 is hydroxylated via a hydroxylase (cytochrome P450 (CYP), polyphenol oxidase (PPO) or other), and decarboxylated by an L-tyrosine/L-DOPA decarboxylase enzyme (TyDC) to dopamine 3 through tyramine 4 or L-3,4-dihydroxyphenylalanine (L-DOPA 5). Next, dopamine 3 is O-methylated by an O-methyltransferase (OMT) and hydroxylated by a hydroxylase to 3,4-dihydroxy-5-methoxy-phenethylamine (3,4-diOH-5-MeO-phenethylamine 6) via 3-methoxy-tyramine (3-MeO-tyramine 7) or 3,4,5-trihydroxy-phenethylamine (3,4,5-triOH-phenethylamine 8). Lastly, two consecutive O-methylations catalyzed by one or more OMTs lead to mescaline 1 through 4-hydroxy-3,5-dimethoxy-phenethylamine (4-OH-3,5-diMeO-phenethylamine 9). 3,4-diOH-5-MeO-phenethylamine 6 was also predicted to be O-methylated probably by an OMT to 3-hydroxy-4,5-dimethoxy-phenethylamine (3-OH-4,5-diMeO-phenethylamine 10) which is thought to be the substrate of tetrahydroisoquinolines (THIQs) in Peyote (FIGS. 1A-1B).

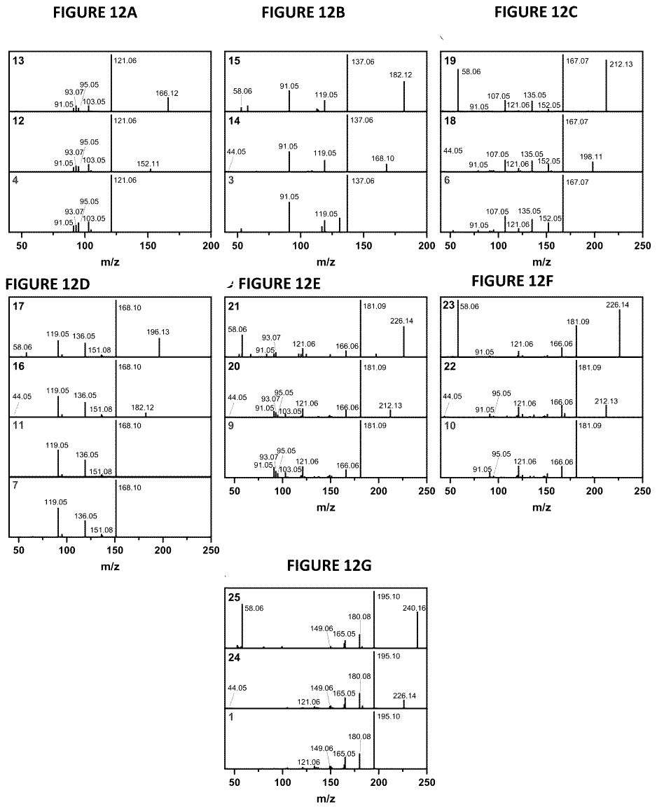

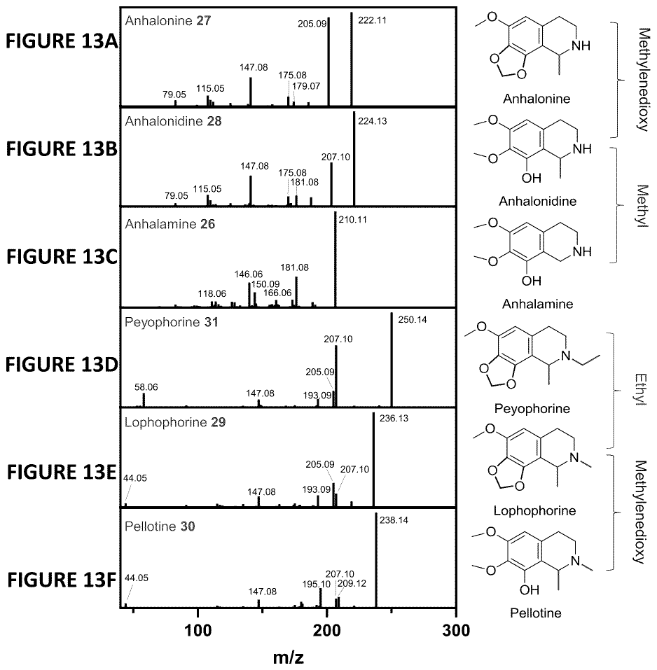

In order to explore this pathway, the inventors first analyzed the metabolic profile of different tissues from Peyote cacti using liquid chromatography tandem mass spectrometry (LC-MS/MS), based on previously identified metabolites in this cactus. The inventors observed most of the metabolites from the predicted pathway (metabolites 1-4, 6, 7, 9 and 10, FIG. 11) as well as some other related molecules. The inventors identified a small peak with similar mass and MS/MS spectrum as 3-MeO-tyramine 7, which the inventors attributed as 4-methoxy-tyramine (4-MeO-tyramine 11, FIGS. 1A and 12D). The inventors further putatively identified N-methylated products of most of these phenethylamines, produced probably via one or more N-methyltransferases (NMTs, metabolites 12-25 FIGS. 1A and 12), and several major THIQs (metabolites 26-31) previously identified in Peyote (FIGS. 1B and 13). The inventors did not observe peaks that could be assigned to 3,4,5-triOH-phenethylamine 8 or its N-methylated derivatives in any of the tissues.

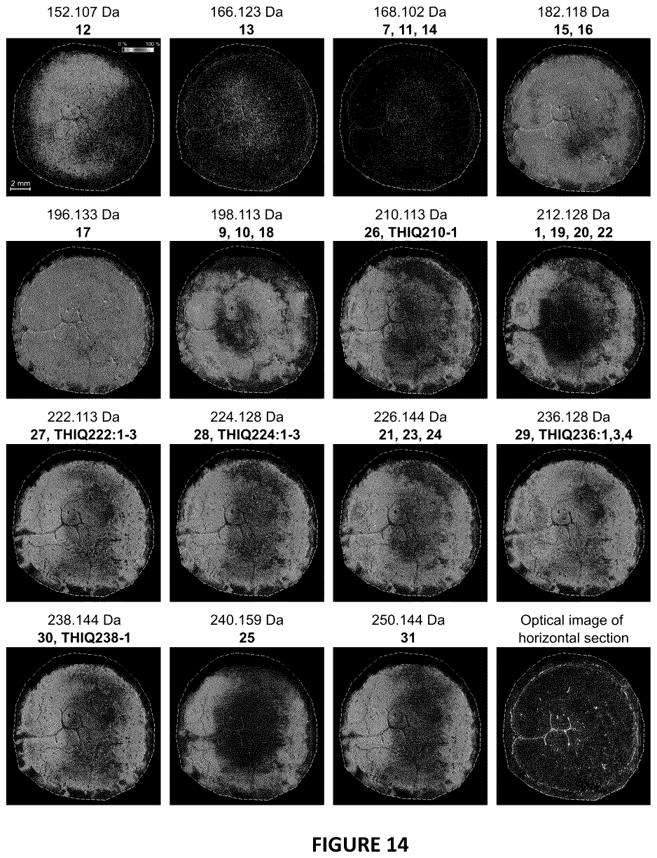

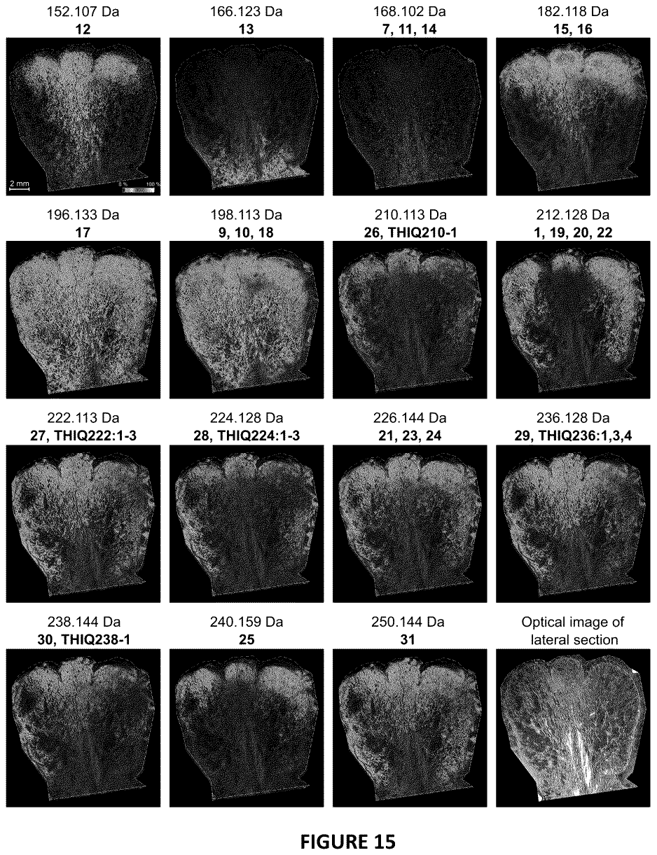

The inventors found in accordance with previous reports that mescaline 1 accumulates mostly in the Peyote top aerial part (button, FIGS. 2A-2B). Using matrix-assisted laser desorption/ionization-mass spectrometry imaging (MALDI-MSI) the inventors further spatially localized mescaline 1 in horizontal and lateral cryo-sections of Peyote. As shown, mescaline 1 and other phenethylamines with m/z [M+H]⁺=212.1281 Da (mainly N-methyl-4-hydroxy-3,5-dimethoxy-phenethylamine 20, and N-methyl-3-hydroxy-4,5-dimethoxy-phenethylamine 22, FIGS. 2C-2G) localize specifically to the button sub-epidermal green tissue (skin) that also contains high density of chlorophyll b (FIGS. 2H-2I). As previously suggested, hordenine 13 was most abundant in the lower parts of the stem and roots compared to the button (FIGS. 2J-2K), exhibiting a unique localization pattern compared to other metabolites in these sections (FIGS. 14-15).

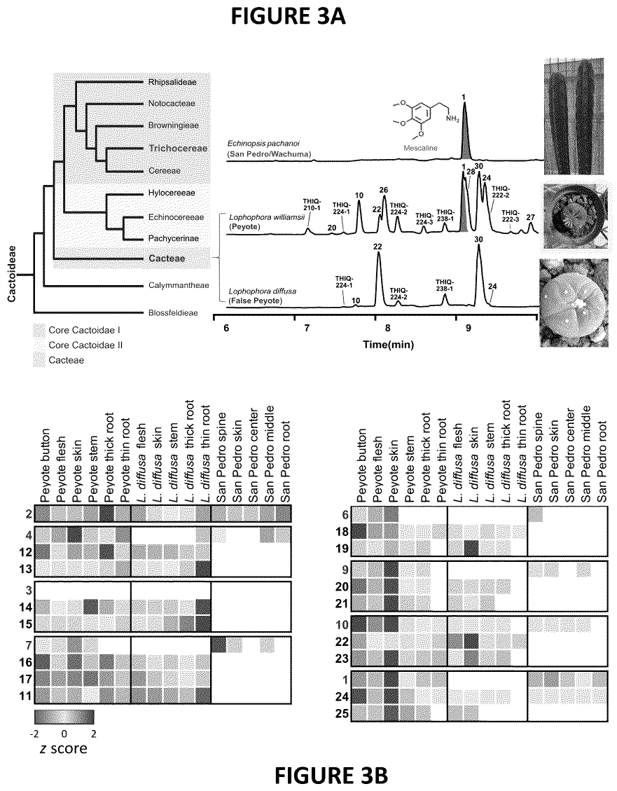

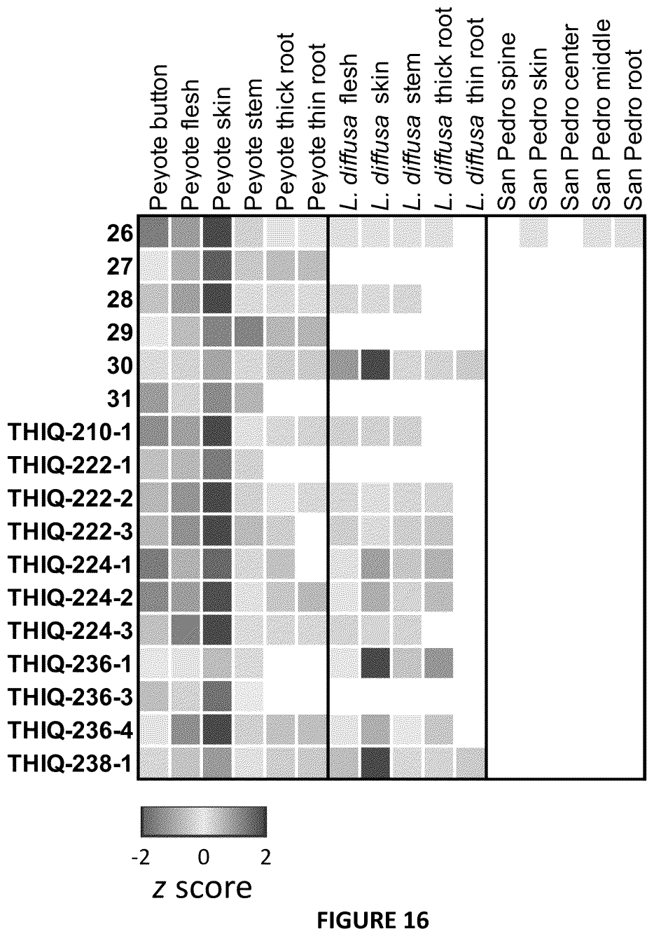

The inventors further analyzed the phytochemistry of two other cacti: Echinopsis pachanoi (also known as Trichocereus pachanoi, San Pedro or Wachuma) which is taxonomically distant from Peyote but also produces large concentrations of mescaline 1 (FIG. 2B), and Lophophora diffusa (L. diffusa), a taxonomically close relative of Peyote that lacks mescaline 1 (FIG. 3A). Each species exhibited unique chemical profiles suggesting possible differences in biosynthetic pathways (FIGS. 3B and 16). For example, the inventors observed that San Pedro accumulates similar metabolites as Peyote for the production of mescaline 1, however, the inventors did not detect N-methylated (apart from N-methylmescaline 24, FIG. 3B) or THIQ (apart from anhalamine 26, FIG. 16) metabolites. In L. diffusa, on the other hand, the inventors identified several THIQs from Peyote, yet the inventors could not detect mescaline 1 nor metabolites 3-9, but rather their N-methylated forms (FIG. 3B). This may suggest very rapid N-methylation activity in L. diffusa which impedes the accumulation of the primary metabolites.

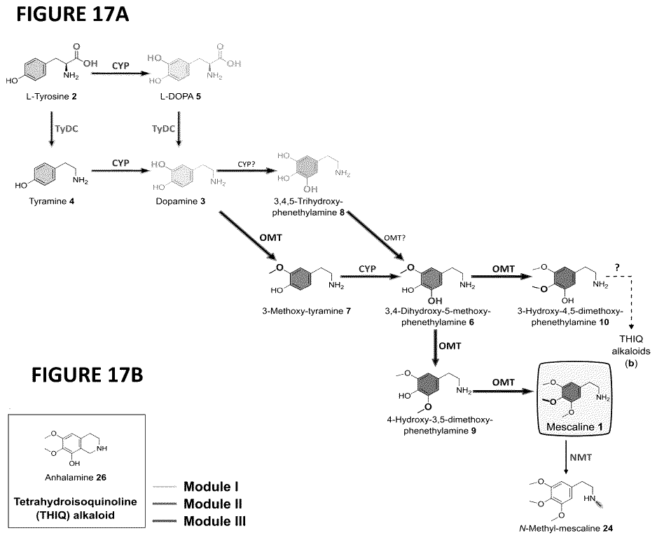

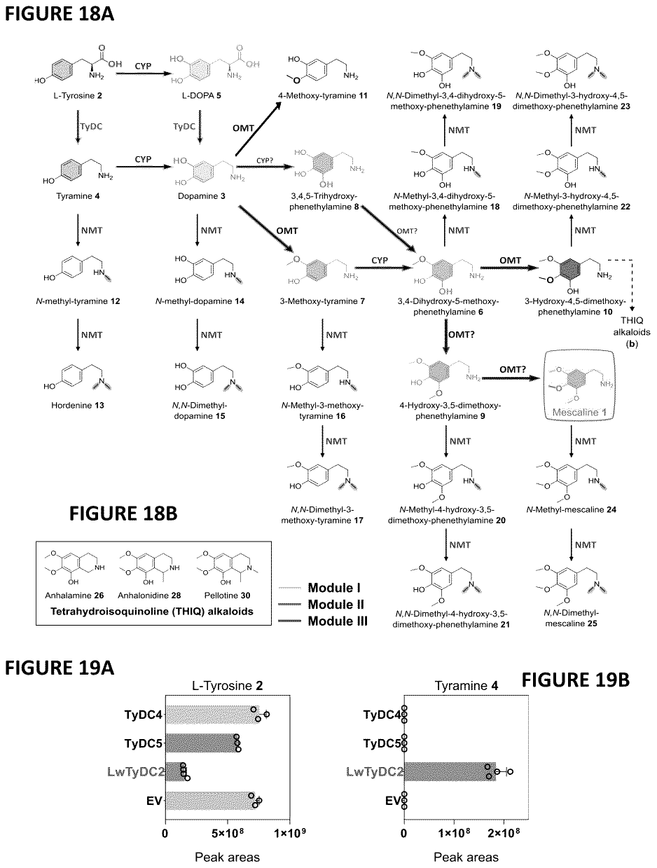

Possible biosynthetic pathways of San Pedro and L. diffusa according to the main observed metabolites appear in FIGS. 17-18, respectively. Notably, in all the species, a larger accumulation of most metabolites from the early steps of the biosynthetic pathway (2-4 and their N-methylated forms) was observed in the roots, while the others were more abundant in the skin and spines (the flesh area around the spines in San Pedro, FIG. 3B). Interestingly, Peyote was the only species of the three that accumulated metabolites with a methylenedioxy group like in anhalonine 27, lophophorine 29, peyophorine 31 (FIG. 16), and other types from the literature. This reaction, commonly catalyzed by CYPs, suggests the presence of a unique enzyme in Peyote compared to the other cacti.

### Example 2

### Peyote Genome Sequencing and Annotation

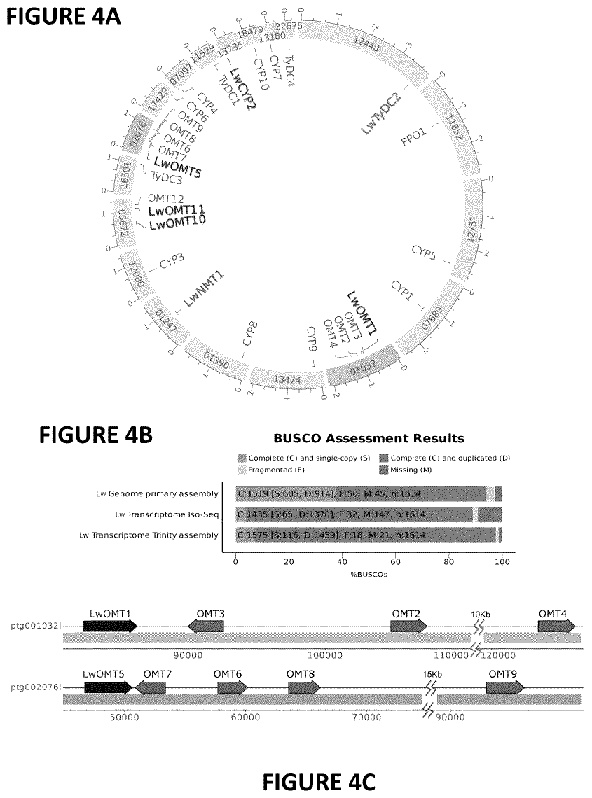

To identify the enzymes responsible for mescaline 1 biosynthesis in Peyote, the inventors obtained a 11× Pacbio HiFi based-assembly. Approximately 37 K contigs were generated (11×3.2 Gb genome size, N50 127 Kb), and genes were annotated using Pacbio Iso-Seq and Illumina paired-end-based transcriptomes from different tissues (FIG. 4A). The BUSCO completeness values were 94.1% for the primary genome assembly, 97.6% for Illumina-based transcriptome, and 88.9% for Pacbio Isoseq (FIG. 4B). Interestingly, two groups of MTs were organized in tandem gene clusters (FIG. 4B).

On the basis of FIG. 1A, the inventors focused in this study on four enzyme families, namely TyDC, CYP, PPO, and MTs. Approximately 5 TyDCs, 270 CYPs, 3 PPOs and 350 MTs were annotated in the Peyote genome. Out of these genes the inventors selected candidates based on relative expression levels in Peyote tissues, favoring those highly expressed in the skin and button, and lowly expressed in the roots. The inventors also considered the protein size, the presence of protein family (Pfam) motifs, and the similarity to functional enzymes involved in secondary metabolite biosynthesis from the literature.

### Example 3

a Single TyDC from Peyote can Catalyze the Decarboxylation of Both L-Tyrosine and L-DOPA

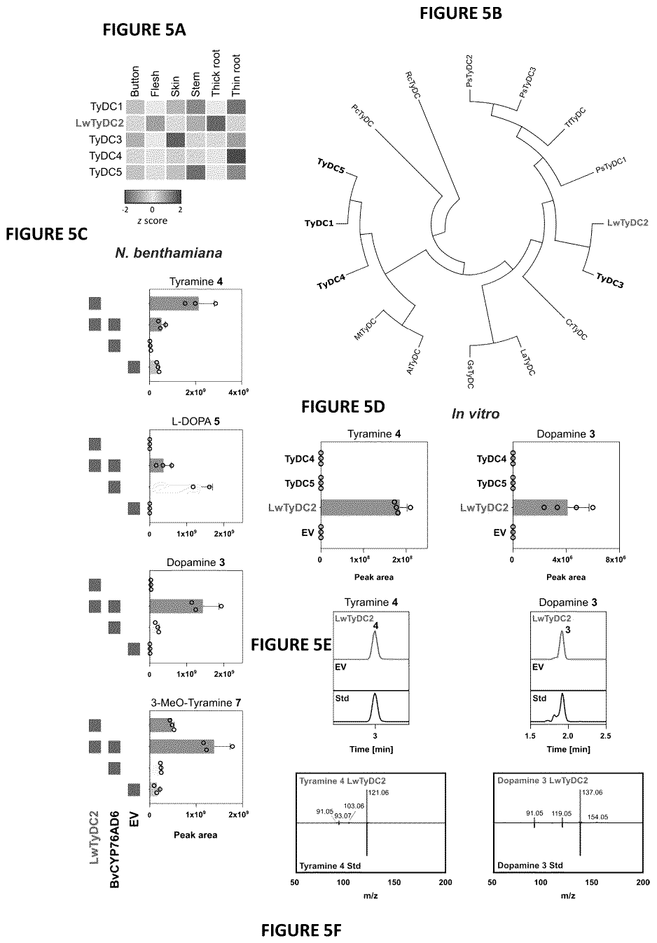

In the first stage, the inventors searched for potential TyDC candidates that catalyze the decarboxylation of L-tyrosine 2 into tyramine 4 and/or L-DOPA 5 into dopamine 3 (FIG. 1A). This enzyme is categorized as aromatic-L-amino-acid decarboxylase (AAADC) and depends on pyridoxal 5-phosphate (PLP). Among the five TyDC genes identified in the genome, TyDC2 exhibited the highest expression levels in all tissues, particularly in thick roots (FIGS. 5A-5B), which aligns with the higher concentrations of metabolites from the early stages of the pathway (FIG. 1A).

Therefore, the inventors initially introduced TyDC2, the most promising candidate, into N. benthamiana leaves through agro-infiltration. As L-tyrosine 2 was naturally present in the plant, the inventors observed a significant increase in the peak areas of tyramine 4 and 3-MeO-tyramine 7 compared to the empty vector (EV), although N. benthamiana itself exhibited a considerable abundance of this metabolite (FIG. 5C). The inventors further co-infiltrated both TyDC2 and BvCYP76AD6, a betalain-related enzyme previously identified in beetroot, which catalyzes the hydroxylation of L-tyrosine 2 into L-DOPA 5. As anticipated, the co-infiltration resulted in a significant accumulation of L-DOPA 5 and a substantial increase in dopamine 3 and its O-methylated product 3-MeO-tyramine 7 (FIG. 5C), suggesting that TyDC2 accepts both L-tyrosine 2 and L-DOPA 5. It is well known that TyDC enzymes often accept both substrates in varying relative activities towards one of the substrates. Interestingly, the phylogenetic analysis in FIG. 5B shows that TyDC2 is closest in amino acid sequence to Pappaver somnferum and Thalictrum flavum TyDCs which have close relative activities towards both L-tyrosine 2 and L-DOPA 5.

Considering the high levels of tyramine 4 naturally present in N. benthamiana and taking into account that TyDCs are typically cytosolic enzymes, the inventors further recombinantly expressed TyDC2, along with TyDC4 and TyDC5 candidates, in E. coli. The inventors then conducted in vitro assays using lysates from E. coli cells transformed with each candidate, utilizing either L-tyrosine 2 or L-DOPA 5 as substrates in the presence of PLP. Among these candidates, only TyDC2 (LwTyDC2) was active showing high catalytic activity for the production of both tyramine 4, and dopamine 3 (FIGS. 5D-5F). In the L-DOPA 5 assays the inventors observed also peaks of L-tyrosine 2 in all the samples coming from the E. coli lysates, and production of tyramine 4 only with LwTyDC2 (FIG. 19). The inventors therefore purified LwTyDC2 and repeated the assay showing similar activities with the pure protein (FIGS. 5E-5F).

### Example 4

### Novel CYP76 and CYP84 Regioselectively Hydroxylate the Two Meta Positions in Mescaline Biosynthesis

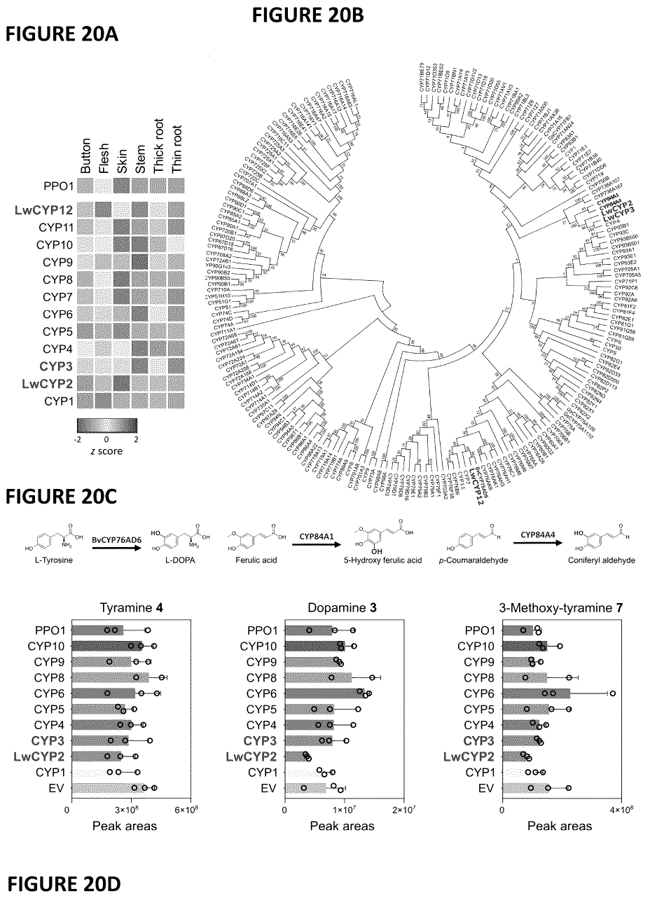

Next, the inventors searched for the hydroxylases. This pathway includes two potential hydroxylation reactions leading to dopamine 3 and two other potential reactions leading to 3,4-diOH-5-MeO-phenethylamine 6 (Modules I and II, respectively, FIG. 1A). The inventors selected twelve CYP candidates based on their relative expression levels in Peyote tissues, (FIG. 20A) and based on similarity to known functional CYPs from the literature (FIG. 20B-20C), and the only highly expressed PPO enzyme (FIG. 20A). Since CYPs are typically found bound to endoplasmic reticulum or inner mitochondrial membranes, and PPOs are typically associated to thylakoid membranes in chloroplasts, the inventors opted to evaluate potential candidates by transiently expressing them individually in N. benthamiana.

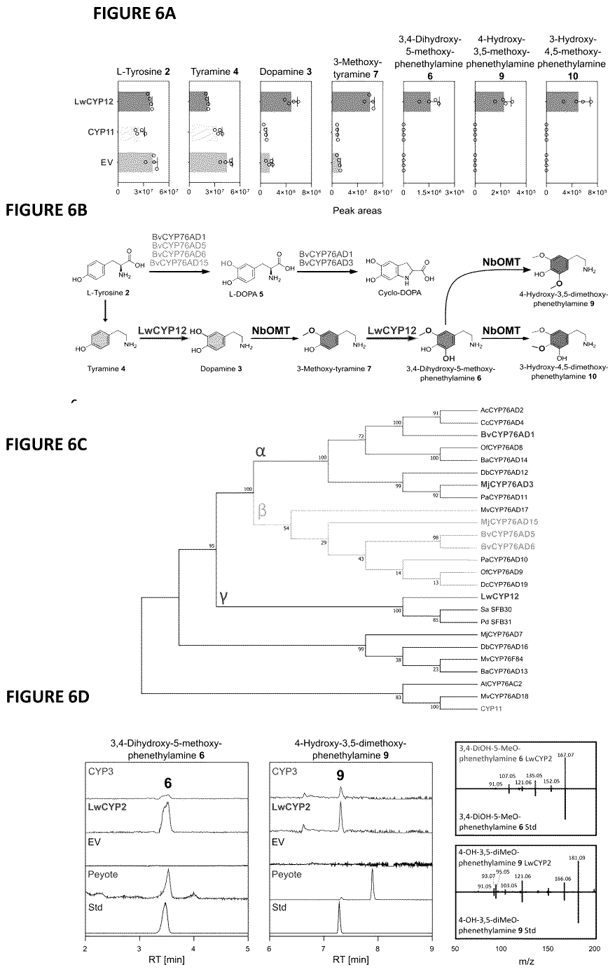

For the hydroxylation step in Module I, the inventors identified three homologues of BvCYP76AD6 (CYP7, CYP11 and CYP12) exhibiting 54%, 51% and 62% sequence similarity, respectively (FIG. 20B). Transient expression of CYP7 by agroinfiltration to N. benthamiana leaves led to high toxicity resulting in tissue necrosis, and was therefore excluded from further characterization. Among the other two candidates, agroinfiltration of CYP12 (LwCYP12) led to a decrease in tyramine 4, increase in dopamine 3 and 3-MeO-tyramine 7, and production of 3,4-diOH-5-MeO-phenethylamine 6, 4-OH-3,5-diMeO-phenethylamine 9 and 3-OH-4,5-diMeO-phenethylamine 10 (FIG. 6A). LwCYP12 therefore, regioselectively catalyzes the two meta hydroxylations (marked in red on FIG. 6B) of both tyramine 4 to dopamine 3 and 3-MeO-tyramine 7 to 3,4-diOH-5-MeO-phenethylamine 6 (FIG. 6B). The observed increase in 3-MeO-tyramine 7, 4-OH-3,5-diMeO-phenethylamine 9 and 3-OH-4,5-diMeO-phenethylamine 10 in these experiments was most likely mediated by one or several endogenous OMTs (NbOMTs) present in N. benthamiana (FIG. 6B).

To validate the enzyme activity observed in N. benthamiana, the inventors co-expressed LwTyDC2 and LwCYP76AD131 in S. cerevisiae yeast and noted the production of tyramine 4 and a minor peak of dopamine 3, accompanied by their putative N-acetylated forms. Consistent with the results in N. benthamiana, the inventors did not observe peaks of L-DOPA 5 in these samples or following expression of only LwCYP76AD131. Incubation of empty vector (EV) transformed yeast with either tyramine 4 or dopamine 3 resulted in the appearance of peaks with similar mass and retention time as N-acetyl-tyramine 32 and N-acetyl-dopamine 33. Identification of N-acetylated metabolites was further confirmed using analytical standards. The detection of dopamine 3 and N-acetyl-dopamine 33 confirms the tyramine 4 hydroxylating activity of LwCYP76AD131 and suggests competition between the expressed Peyote enzymes and an endogenous N-acetylating enzyme in yeast for the newly formed phenethylamines.

Existing literature demonstrates that the CYP76AD-subfamily within betalain-producing Caryophyllales can be categorized into three distinct clades. Functionally characterized enzymes in the β-clade (CYP76AD5/6/15) specialize in catalyzing the hydroxylation of L-tyrosine 2, while enzymes in the α-clade (CYP76AD1/3) catalyze both L-tyrosine 2 hydroxylation and L-DOPA 5 oxidative cyclization, resulting in the formation of cyclo-DOPA (FIGS. 6B and 6C). Notably, LwCYP12 is a member of the y-clade (FIGS. 6B and 6C), a category that, prior to the discovery of this enzyme, lacked any known function.

The inventors further infiltrated all the other CYP candidates without supplementation of any substrates. However, none of the candidates demonstrated the production of L-DOPA 5, nor did they exhibit a significant increase in dopamine 3, tyramine 4 or 3-MeO-tyramine 7 (FIG. 20D).

Next, the inventors agroinfiltrated CYP-10 supplementing the plants with 50 μg ml⁻¹ of 3-MeO-tyramine 7, and found that CYP2, and to a lesser extent also CYP3, showed low-abundance peaks of 3,4-diOH-5-MeO-phenethylamine 6. None of the infiltrated enzymes produced peaks of 3,4,5-triOH-phenetethylamine 8. Increasing the substrate concentration resulted in a higher concentration of this peak and the appearance of 4-OH-5-diMeO-phenethylamine 6 (FIG. 6D). The observed increase in O-methyl products of dopamine 3 and 3,4-diOH-5-MeO-phenethylamine 6 in these experiments was most likely catalyzed by one or several endogenous OMTs present in N. benthamiana (FIG. 6B). It is worth noting that CYP2 (LwCYP2) and CYP3 belong to the same clade as CYP84A1 (FIGS. 20B-20C), which has been previously identified as a ferulic acid hydroxylase. This relationship suggests a possible explanation for the comparable regioselectivities, and catalytic functions observed with LwCYP2 and CYP3.

The inventors further individually expressed LwCYP76AD131 and LwCYP84A220 in yeast and supplemented them with 3-MeO-tyramine 7 as the substrate. Following two days from galactose induction, the inventors observed peaks of 3,4-diOH-5-MeO-phenethylamine 6 in the samples expressing LwCYP76AD131. Since the peaks observed with LwCYP84A220 could not be confirmed via MS/MS due to their low abundance, the inventors inferred that LwCYP76AD131 exhibited a superior catalytic activity for the production of 3,4-diOH-5-MeO-phenethylamine 6. This confirmed the second hydroxylating activity of LwCYP76AD131 towards 3,4-diOH-5-MeO-phenethylamine 6.

### Example 5

### Peyote Catalyzes Multiple N-Methylations Via a Single NMT

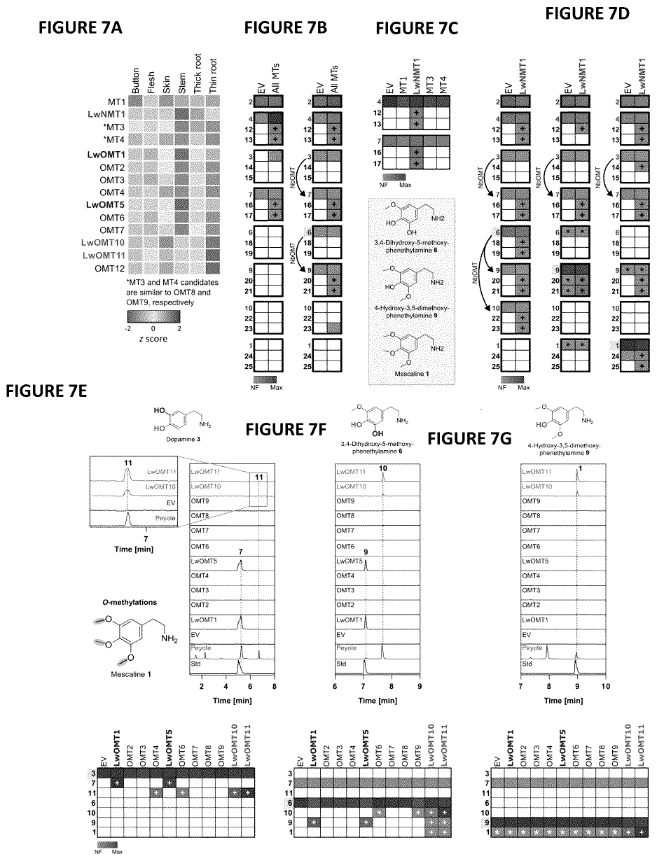

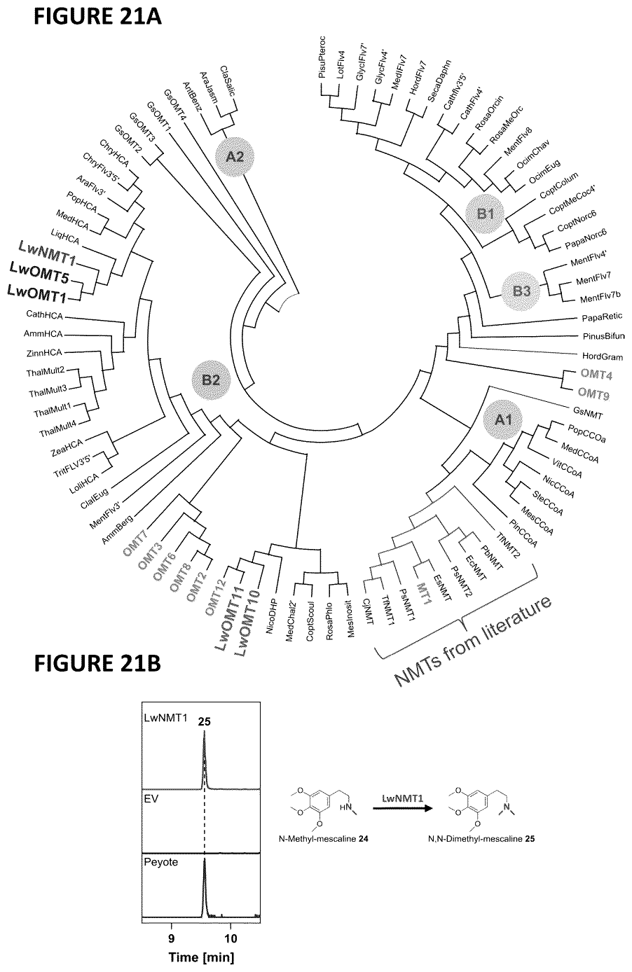

Lastly the inventors searched for MTs. These ubiquitous enzymes transfer a methyl group from S-adenosyl L-methionine (SAM) to a nucleophilic acceptor leading to S-adenosyl-L-homocysteine and a methylated molecule. The inventors first selected four candidates according to protein sequence similarity to known OMTs and NMTs (MT1-MT4, FIGS. 7A and 21A) and transiently expressed a combination of the four genes in N. benthamiana leaves with and without supplementation of 3,4-diOH-5-MeO-phenethylamine 6. As depicted in FIG. 7B, one or more of the MT candidates demonstrated N-methylation activity (indicated by “+”). Next, the inventors individually infiltrated each of the MT candidates and identified MT2 as the active NMT (LwNMT1, FIG. 7C). Furthermore, by supplementing with 3,4-diOH-5-MeO-phenethylamine 6, 4-OH-3,5-diMeO-phenethylamine 9, or mescaline 1, the inventors observed broad N-methylation and N,N-dimethylation activity resulting in the production of numerous N-phenethylamine products catalyzed by this single enzyme (FIG. 7D). The inventors further supplemented plants with N-methyl-mescaline 24 to demonstrate that the second N-methylation is catalyzed by LwNMT1 and not by the plant (FIG. 21B). The inventors also expressed LwNMT1 recombinantly in E. coli and confirmed its activity in vitro using lysates in the presence of SAM and either dopamine 3, 3,4-diOH-5-MeO-phenethylamine 6, or 4-OH-3,5-diMeO-phenethylamine 9 as substrates.

### Example 6

### Two Groups of OMTs Participate in the Production of Mescaline 1

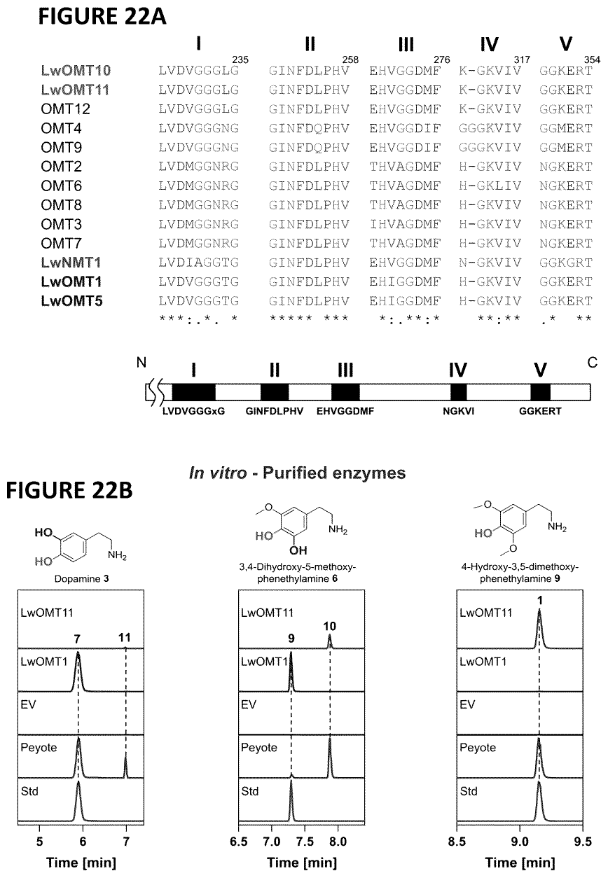

Considering the O-methylation activity mediated by the endogenous OMTs from N. benthamiana (3 to 7, 6 to 9 and 6 to 10, FIGS. 7B-7C), the inventors proceeded to express ten new candidate enzymes recombinantly in E. coli and evaluate their activities in vitro (OMT1-OMT7 and OMT10-OMT12, FIG. 7A). The inventors also included MT3 and MT4 among these candidates (referred here as OMT8 and OMT9, respectively), as they were highly expressed and clustered together with other known OMTs (FIG. 21A). All these candidates possessed conserved amino acid sequence regions characteristic of OMTs, as previously proposed (FIG. 22A). The enzymes OMT10-OMT12 were located in tandem in the genome (FIG. 4A) and had very similar sequences (OMT11 and OMT12 were identical).

Next, the inventors tested lysates from E. coli transformed with OMT1-OMT11 candidate enzymes in the presence of SAM and either dopamine 3, 3,4-diOH-5-MeO-phenethylamine 6, or 4-OH-3,5-diMeO-phenethylamine 9. Here, the inventors identified two distinct groups of enzymes: OMT1 and OMT5 (LwOMT1 and LwOMT5 marked in blue) which O-methylated the two meta hydroxyl groups in mescaline 1, and OMT10 and OMT11 (LwOMT10 and LwOMT11 marked in purple) which targeted the para hydroxyl (FIGS. 7E-7G for substrates 3, 6 and 9, respectively; the specific O-methylated hydroxyls were highlighted in blue or purple on each substrate molecule according to the type of enzyme). Remarkably, the inventors found that LwOMT10 and LwOMT11 enzymes also generated small quantities of mescaline 1 with 3,4-diOH-5-MeO-phenethylamine 6 as the substrate. This occurrence is likely attributed to a secondary minor activity of these enzymes on the resulting 3-OH-4,5-diMeO-phenethylamine 10. Interestingly, according to the phylogenetic analysis the active MTs resemble most hydroxycinnamic acid (HCA) and simple phenol OMTs (FIG. 21A, assignment of clusters was according to Lam et al., 2007; Genome). The inventors further purified LwOMT1 and LwOMT11 and repeated the assays showing similar activities with the pure proteins (FIG. 22B).

### Example 7

Regioselectivities of LwOMT1, LwOMT11 and LwNMT1 Derive from Spatial Proximity of SAM to the Specific Target Sites

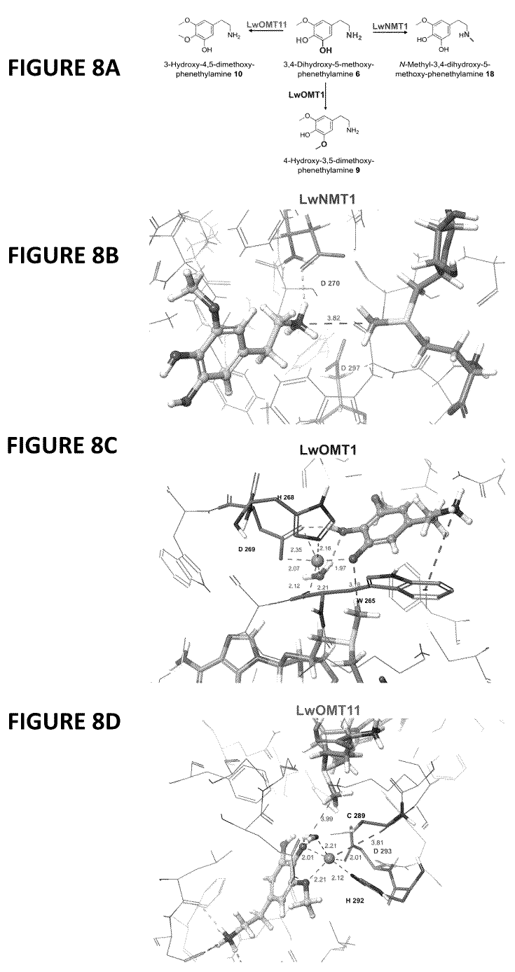

The functional characterization data for the active MTs demonstrated their regioselectivity towards specific hydroxyl or amine groups. For instance, all three enzymes were capable of methylating 3,4-diOH-5-MeO-phenethylamine 6, but each enzyme targeted the methylation of a distinct group (FIG. 8A). The phylogenetic analysis also revealed that LwNMT1 did not cluster with other functionally characterized NMTs from the literature, but instead it exhibited a closer sequence homology to LwOMT1 and LwOMT5 (71% similarity to both OMTs, FIG. 21A). Therefore, in order to elucidate the molecular basis for this regioselectivity, the inventors employed molecular modeling and conducted docking studies of the metabolite in each of the enzymes.

Backbone alignment RMSD values for LwNMT1, LwOMT1 and LwOMT11 to templates were 3.20, 1.65 and 2.14 Å, respectively. For all the enzymes, the proximity of at least one Asp/Glu was necessary as a base for proton withdrawal and facilitating nucleophilic attack of substrate on the SAM methyl group. The SAM binding pockets of newly built models coincided spatially with those of the template MTs and accommodated SAM itself without major steric clashes as also did the substrates. Correctness of SAM placement was supported by retaining interactions observed by experimentally determined structures of SAM-dependent MTs such as those with PDB ID 2AN4, 6LFE and others. These include Asp/Glu side chain carboxylate interaction with the ribose residue of SAM, and carboxylate group interaction with the 6-aminoadenine group.

In the case of LwNMT1 the initial placement of 3,4-diOH-5-MeO-phenethylamine 6 was rejected since the side chain carboxylate anion essential for catalytic activity was not in the proximity of the S-methyl and ammonium groups. Taking into account the sequence similarity between LwNMT1 and LwOMT1 the inventors hypothesized that LwNMT1's substrate pocket was homologous to that of LwOMT1. Therefore, 3,4-diOH-5-MeO-phenethylamine 6 was moved to that site and orientated accordingly (FIG. 8B).

However, when the inventors tried to place the substrate molecules in LwOMT1 and LwOMT11 more accurately by docking, and to choose the most favorable pose based on the MM-GBSA free energy of binding calculation, the inventors unexpectedly obtained the complexes preferably with beta-amino group in the proximity of SAM pointing on possible N-methylation. This led us to assumption that the reaction center was different for these OMTs. The inventors therefore hypothesized based on both experimental structural data and theoretical studies that the aromatic O-methylation in this pathway is magnesium-mediated. Since the accurate geometry of catalytic centers require taking into account the electronic effects and could hardly be achieved within the MM paradigm, the inventors applied the QM/MM conception.

For LwOMT1, the magnesium ion can be considered here as 6-coordinated by the imidazolic ring of the H268 residue, a water molecule, the negatively charged oxygen atom of the catecholate anion, the carboxylic group of D269 (both oxygens) and the carbonyl oxygen of the W265 backbone (FIG. 8C). The angle between the pyramidal vertices (imidazolic sp2-hybridized nitrogen and water oxygen atoms) and magnesium is 169.4°. The active site of LwOMT11 was characterized by magnesium cation quasi-6-coordinated by a deprotonated phenolic oxygen atom, a methylated oxygen in the ortho-position, a water molecule, imidazolic sp2-hybridized nitrogen, the carboxylate oxygen of D293 and weak interaction with the C289 side chain carbonyl oxygen (d=3.81 Å, FIG. 8D).

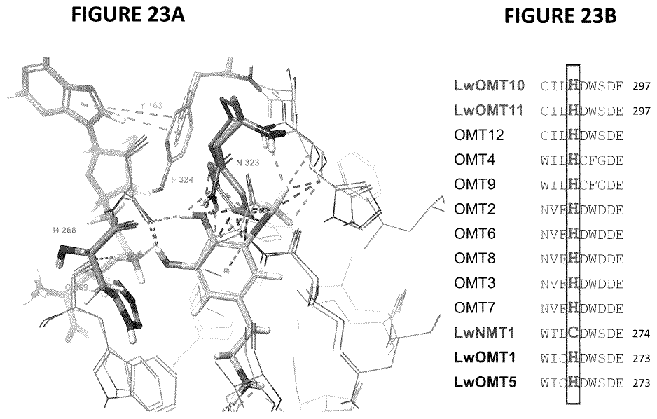

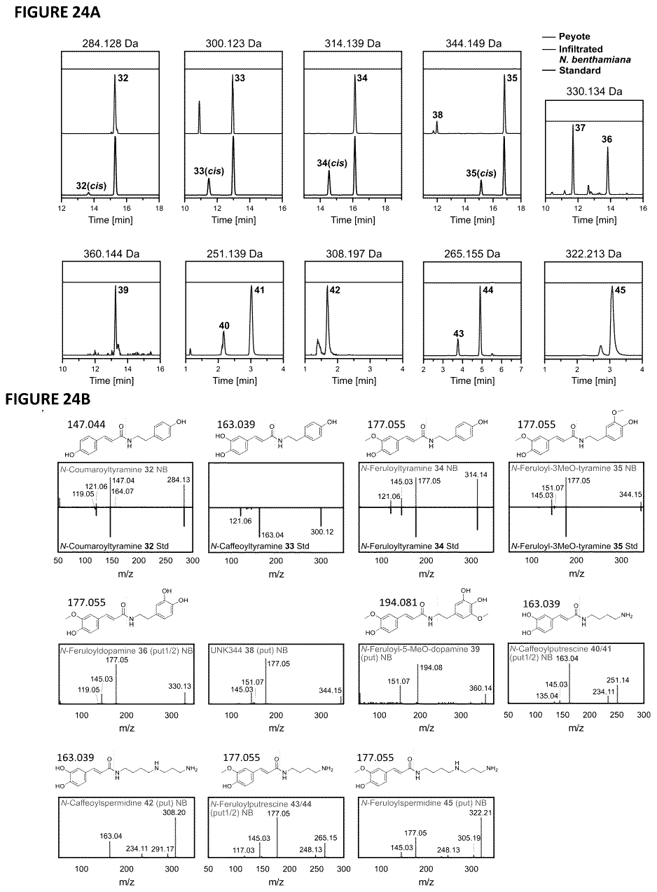

To further shed light on the differences in regioselectivity between the highly similar LwOMT1 and LwNMT1 (FIG. 21A), the inventors performed backbone alignment of these enzymes. The RMSD value between these enzymes aligned by their backbones was 1.19 Å. The obvious difference in the substrate binding site of LwOMT1 is the presence of the bulky amino acid F324 instead of N323 close to the SAM binding pocket, which enables accommodation of the substrate's 2-ethylammonium “tail” but forms steric clashes with the poly-substituted benzene “head” (FIG. 23A), and of Y163 instead of F162. These substitutions hinder the proximity of SAM from the OH-groups for effective methylation. On the contrary, the catalytic site of LwNMT1 does not have a histidine residue which was predicted to be essential for the reaction complex formation (H268 in LwOMT1, FIG. 23B). Therefore, according to the inventors' prediction, the amino acid substitutions in the catalytic site of LwNMT1 (Y163 and C269) versus LwOMT1 (F162 and H268) make it more available for hydrogen bond donor-orientation, and less capable of coordinating the protons or magnesium ion for O-methylation.

### Example 8

Engineering of the Mescaline Pathway in N. benthamiana

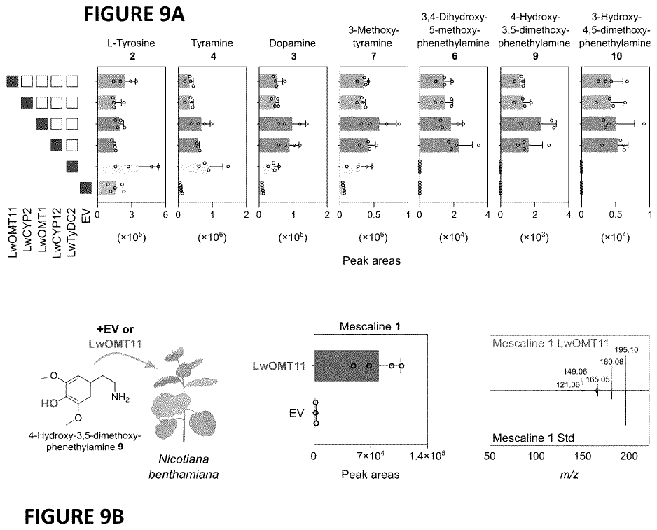

Taking advantage of the existing pool of L-tyrosine 2 in N. benthamiana, the inventors decided to transiently express in planta the entire pathway towards mescaline 1 without supplementation of any substrates. The inventors therefore co-expressed stepwise all the functionally characterized enzymes from Peyote generating all the intermediates towards mescaline 1, yet without attaining the final product (FIG. 9A).

Further supplementation of 4-OH-3,5-diMeO-phenethylamine 9 to plants expressing LwOMT11 resulted in the production of mescaline 1 proving in planta activity of this enzyme (FIG. 9B). Interestingly, N. benthamiana did not show substantial 4-O-methylation activity on 3,4-diOH-phenethylamine 6, suggesting that the plant does not contain an LwOMT11 orthologue (the mescaline 1 peaks observed with the EV in FIG. 9B result from low concentration contamination present in the infiltrated standard).

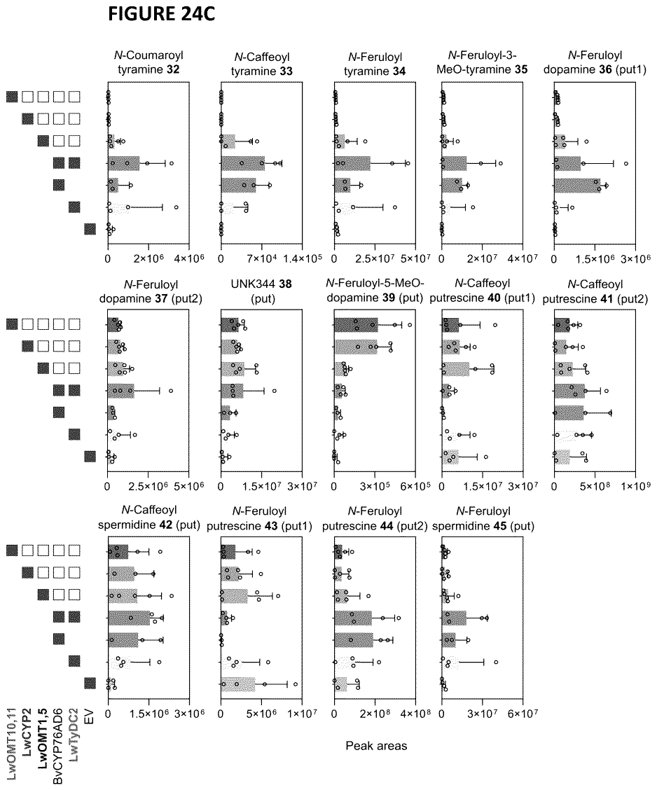

The inventors hypothesized that the expressed Peyote enzymes in N. benthamiana may also be active on metabolites, such as phenylpropanoids, hydroxycinnamic acids, and others, which share structural similarities with intermediates from the mescaline 1 pathway. This could lead to a competitive scenario, with these enzymes being utilized in multiple pathways. Considering also that N. benthamiana naturally produces hydroxycinnamic acid amides (HCAAs), the inventors predicted that the plant may utilize the phenethylamine intermediates from the mescaline 1 pathway, like tyramine 4, dopamine 3, 3-MeO-tyramine 7, and others, to synthesize more of these HCAAs. The inventors therefore screened the LC/MS data of the infiltrated N. benthamiana samples and identified several HCAAs (metabolites 32-45, identification was according to Li et al., 2018; Anal. Chem.) with concentration changes linked to the stepwise expression of the enzymes from Peyote (FIG. 24). Notably, derivatives of tyramine 4 (HCAAs 32-34) exhibited peak areas that paralleled those of tyramine 4 (FIGS. 24C versus 9A). Additionally, a putative HCAA, identified as N-feruloyl-5-methoxy-dopamine (N-Feruloyl-5-MeO-dopamine 39 FIG. 24B), displayed a similar trend of peak areas as 3,4-diOH-5-MeO-phenethylamine (FIGS. 24C versus 9A). Nevertheless, it is worth noting that certain samples within the same treatment displayed variable peak areas (FIG. 24C). Furthermore, the inventors detected several other differential metabolites that the inventors couldn't assign with confidence.

These results provide further evidence of the promiscuity of certain enzymes and highlight potential interactions between intermediates from these pathways, suggesting a complex network of metabolic flux in N. benthamiana. The presence of branch point metabolites may explain the lack of phenethylamine intermediate accumulation in the plant, as these metabolites likely serve as critical intersections where different biosynthetic pathways converge and compete for utilization. Interestingly, the inventors did not observe any of the identified HCAAs in Peyote (FIG. 24A).

### Example 9

Orthologues in Other Cacti Provide Clues about Mescaline Pathway Evolution

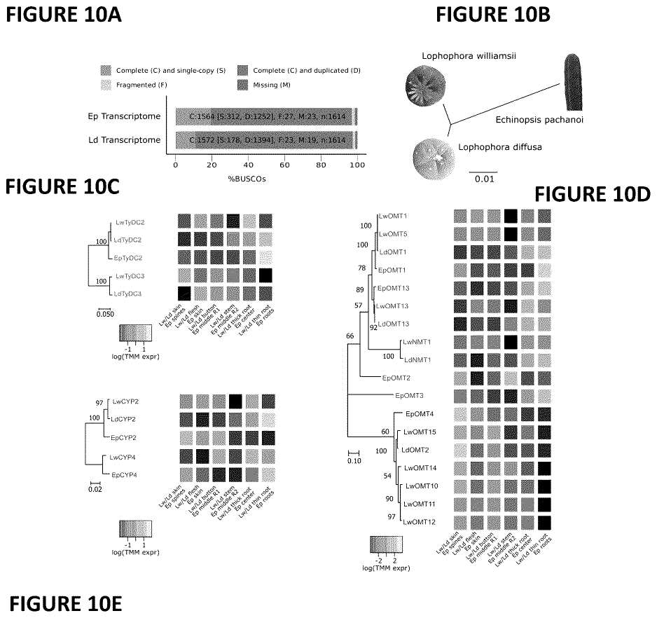

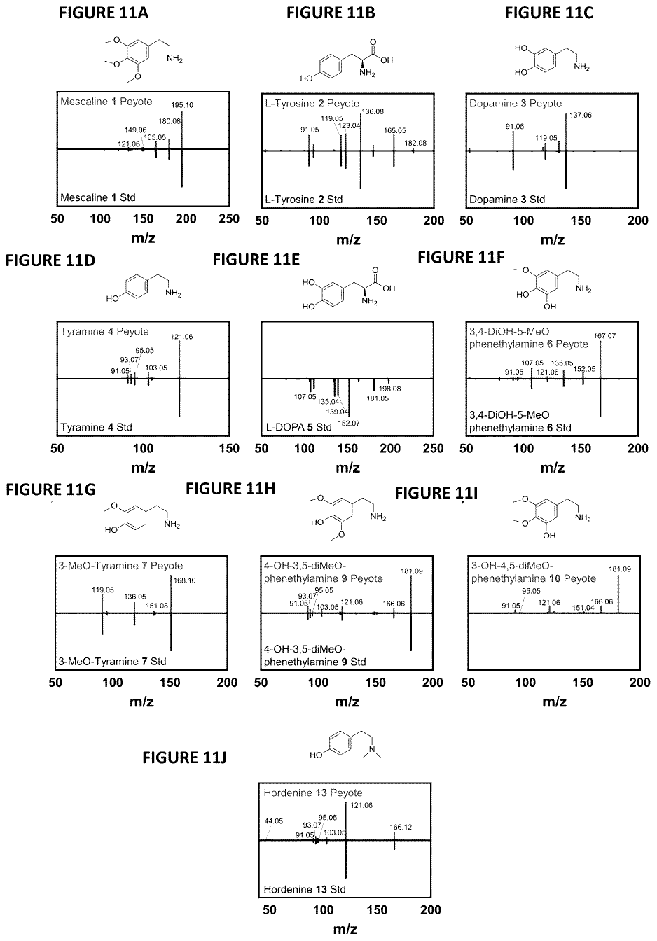

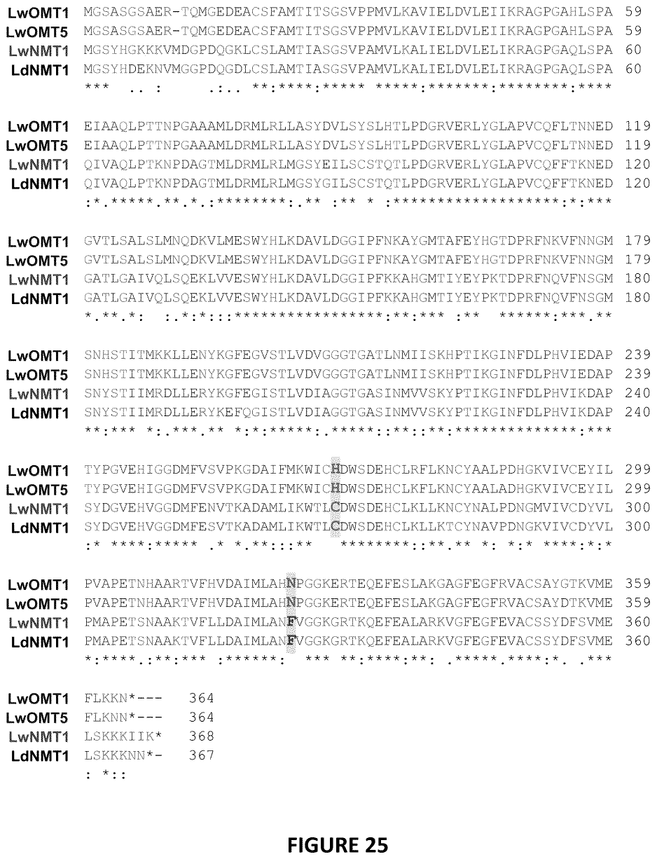

Understanding the similarities and differences in pathway enzymes is crucial for unraveling the evolution of the mescaline 1 pathway. The inventors sequenced and assembled San Pedro and L. diffusa transcriptomes using Illumina Paired-End sequencing (BUSCO completeness values of 96.9% and 97.4% respectively, FIG. 10A). After annotation, whole-proteome phylogeny was performed to quantify the evolutionary distance between the three cacti, showing the divergence between Lophophora and Echinopsis genera (FIG. 10B). Using Orthofinder the inventors identified orthologues of LwTyDC2, LwOMT1, LwOMT5, LwOMT10, LwOMT11 and LwCYP2 (FIGS. 10C-10E) both in L. diffusa and San Pedro. However, while the orthologues from L. diffusa showed relatively large expression, those from San Pedro were lowly expressed, suggesting that the pathway in San Pedro may consist of different enzymes. Interestingly, the inventors identified in L. diffusa one enzyme similar to LwNMT1 (LdNMT1), which is missing in San Pedro (FIG. 10D). This corresponds with the absence of N-methylated products in this cactus (FIG. 3B). LdNMT1 also lacks the histidine that was predicted to be essential for magnesium coordination in the active OMTs (H268 in LwOMT1 and LwOMT5, FIG. 25), and it contains the phenylalanine identified in LwNMT1 that forms steric clashes hindering the proximity of SAM from the hydroxyls of the substrate for effective methylation (F324 in LwNMT1 and LdNMT1, FIG. 25). These results suggest a similar mechanism of N-methylation for LdNMT1.

### Example 10

### In Vivo Reconstruction of the Mescaline Pathway

The inventors next attempted to reconstruct the mescaline 1 pathway by co-expressing LwTyDC2, LwCYP76AD131, LwOMT1 and LwOMT11 enzymes in yeast. Following two days of galactose induction, the observed peaks of tyramine 4 and dopamine 3, in addition to their respective N-acetylated forms both in the pellet and the supernatant of the transformed yeast. However, no other intermediates from the pathway were detected. Following the detection of N-acetylated metabolites the inventors incubated EV transformed yeast with individual intermediates from the pathway to account for potential N-acetylation by a yeast N-acetyltransferase. Following two days of protein induction the inventors could detect all N-acetylated intermediates from the pathway (metabolites 32-36). Consistent with these findings, an arylalkyl amine N-acetyltransferase from S. cerevisiae (scAANAT, GenBank accession number 549826) has been reported to be functional on several phenethylamines, including mescaline 1, dopamine 3, tyramine 4 and 3-MeO-tyramine 7, suggesting it may be responsible for the observed activity.

To confirm the in vivo activity of the enzymes, the inventors further supplemented yeast co-expressing the four enzymes from Peyote with each pathway intermediate individually. Subsequently, depending on the supplemented substrate, the inventors detected the phenethyl products tyramine 4, dopamine 3, 4-OH-3,5-diMeO-phenethylamine 9, 3-OH-4,5-diMeO-phenethylamine 10, and mescaline 1. Additionally, the inventors observed the appearance of N-acetylated products, including N-acetyl tyramine 32, N-acetyl-dopamine 33, N-acetyl-3-MeO-tyramine 34, N-acetyl-4-OH-3,5-diMeO-phenethylamine 36, and two newly observed metabolites putatively identified as N-acetyl-3-OH-4,5-diMeO-phenethylamine 37 and N-acetyl-mescaline 38. Following incubation with 3-MeO-tyramine 7, only a low abundance peak of N-acetyl-3,4-diOH-5-MeO-phenethylamine 35 was detected that couldn't be verified via MS/MS. Overall, the production of the phenethylamine intermediates and their N-acetylated forms by yeast co-expressing the Peyote enzymes confirms the enzymes' activity. However, the presence of N-acetylation competition possibly by scAANAT and potentially low catalytic activity of specific enzymes prevented in vivo synthesis of mescaline 1. This highlights the complexity in future engineering efforts targeting the mescaline 1 pathway in yeast, suggesting that knocking down scAANAT may be necessary to enable its production.

### Discussion

Hallucinogenic cacti have played a significant role in the cultural and religious practices of indigenous communities for thousands of years. However, the increasing recreational use, unsustainable harvesting, and slow growth rates of these cacti pose a potential risk of depleting the natural source of mescaline 1. To safeguard its future development as a potential next-generation neuropsychiatric drug, exploring sustainable production methods becomes imperative, ensuring responsible utilization while preserving the ecological balance for generations to come. The elucidation of the biosynthetic pathway of this metabolite is, therefore, a crucial initial step in this endeavor.

The presence of mescaline 1 and other intermediates from the pathway has been reported in various cacti, especially from the Cacteae and Trichocereae families. This suggests a dominant evolutionary advantage of these metabolites to the plants, possibly serving as a defensive mechanism against herbivores, as indicated by the extreme bitterness of Peyote buttons. Notably, several early intermediates from this pathway, including L-tyrosine 2, tyramine 4, dopamine 3 and 3-MeO-tyramine 7 are also present in various other plant species, including N. benthamiana, and share structural similarities with common metabolites and orthologous enzymes found in other pathways like phenylpropanoid and HCAA. However, mescaline 1 has not been identified in any other plants outside the Cactaceae family. In this study, the inventors provide valuable insight into the limited distribution of this metabolite in plants and its selective accumulation in specific cacti.

The inventors successfully identified four distinct sets of enzymes accountable for five out of the six biosynthetic stages involved in mescaline 1 production. For the missing step, the inventors observed an elevation in 3-MeO-tyramine 7 levels upon the expression of CYP7, although this led to toxic effects in N. benthamiana. The current phylogenetic analysis revealed CYP7's closest resemblance to BvCYP76AD6, yet it exhibited no production of L-DOPA 5. The assessment of the predominant PPO in Peyote also indicated a lack of activity. These suggest the plausibility of an alternative hydroxylase catalyzing this step, potentially a yet undisclosed CYP or dioxygenase. The inventors have recently identified two additional CYP candidates, displaying greater similarity to BvCYP76AD6 (PB.17486 and PB.18251 at 51% and 62%, respectively), and the inventors are currently in the process of functionally testing them. Notably, specific enzymes such as LwCYP2, LwOMT10, and LwOMT11, which are absent in N. benthamiana, may explain the absence of mescaline 1 in this plant despite the presence of 3-MeO-tyramine 7. Moreover, the identification of orthologous enzymes in San Pedro and L. diffusa reinforces this observation.

The stepwise expression of active enzymes in N. benthamiana only yielded low abundance peaks of 3,4-diOH-5-MeO-phenethylamine 6, and 4-OH-3,5-diMeO-phenethylamine 9, impeding the continuation of the pathway towards mescaline 1. This hindrance might be attributed to competition between HCCA or other metabolite classes and the mescaline 1 pathways in N. benthamiana, diverting intermediates away from mescaline 1. The absence of HCAA metabolites (32-45) in Peyote (FIG. 24A) suggests that this type of competition is absent in the cactus. Alternatively, the localization of 3,4-diOH-5-MeO-phenethylamine 6 and other intermediates might render them toxic in N. benthamiana, leading to their sequestration into organelles like the vacuole, limiting their availability to the overexpressed enzymes. It is also plausible that the enzymes in this pathway function relatively slowly in the cacti, and that the high abundance of mescaline 1 found in these cacti results from accumulation over several months or even years, as it is known that mescaline 1 concentration in Peyote depends on the maturity and/or size of the cactus.

In light of the current findings, the presence of LwNMT1 in Peyote and its corresponding orthologue in L. diffusa, align with the occurrence of N-methylated metabolites in both cacti, and their absence in San Pedro (FIG. 3B). It has been proposed that N-methylation in plants serves as a protective strategy against the potential toxicity of some specialized metabolites. Consequently, this observation suggests that San Pedro may have evolved an alternative mechanism to address this challenge. LwNMT1 potentially emerges as a pivotal regulator of this pathway, exerting a diversion of intermediates and end-products away from the primary route (FIG. 1A). It is plausible, therefore, that while L. diffusa synthesizes mescaline 1, this compound, alongside other pathway intermediates, fails to accumulate within the cactus due to a highly active NMT enzyme (FIG. 18). Particularly noteworthy is the remarkable sequence similarity between this enzyme and the active LwOMT1 and LwOMT5, which exhibit distinct regioselective methylation patterns determined by the proximity of the SAM methyl donor to the targeted functional group. These distinctions chiefly arise from the absence of the histidine motif in LwNMT1, a critical element for magnesium coordination (FIG. 23B). Additionally, LwNMT1 features two Asp/Glu residues that play a pivotal role in the effective withdrawal of protons from NH3⁺, along with bulky groups that engender steric clashes, hindering substrate coordination for O-methylation (FIGS. 8B and 23A). Consequently, further exploration into LwNMT1 and its counterpart from L. diffusa stands to offer invaluable insights into the evolutionary dynamics of this pathway across these cacti.

By connecting these vital discoveries, the current study advances the comprehension of mescaline 1 biosynthesis and presents promising avenues for sustainable production and responsible utilization of this invaluable natural resource. To gain a deeper understanding of the evolutionary history of phenethylamines in various cacti, particularly the accumulation of mescaline 1 in Lophophora and Echinopsis genera, further investigations involving genetic and metabolomic profiling of intermediate members within the family are necessary. These studies will significantly contribute to a comprehensive grasp of the biosynthetic pathways of mescaline 1 and related compounds. The preservation of hallucinogenic cacti and their biosynthetic potential is critical for the development of mescaline-based treatments and the protection of cultural and ecological heritage.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. An isolated DNA molecule comprising a nucleic acid sequence having at least 85% identity to SEQ ID NO: 1, or any combination thereof.

2. The isolated DNA molecule of claim 1, wherein said nucleic acid sequence encodes a protein being a decarboxylase.

3. An isolated DNA molecule comprising a nucleic acid sequence having at least 80% identity to any one of SEQ ID Nos: 5, 6, 15, or any combination thereof.

4. The isolated DNA molecule of claim 3, wherein said nucleic acid sequence encodes a protein being a hydroxylase.

5. A plasmid or an agrobacterium comprising the isolated DNA molecule of claim 1.

6. The plasmid or agrobacterium of claim 5, further comprising an additional nucleic acid sequence having at least 75% identity to any one of SEQ ID Nos: 44, 48, 53, 54, or any combination thereof, and optionally wherein said additional nucleic acid sequence encodes a protein being a methyltransferase.

7. An isolated polypeptide encoded by the isolated DNA molecule of claim 1.

8. The isolated polypeptide of claim 7, comprising an amino acid sequence with at least 97% identity to SEQ ID NO: 28 or with at least 96% identity to any one of SEQ ID Nos: 32, 33, and 42, and optionally wherein said isolated polypeptide is characterized by having an activity of releasing a carboxyl group from a substrate, thereby releasing CO₂, or is characterized by having an activity of introducing a hydroxyl group into a substrate.

9. A transgenic cell comprising the isolated DNA molecule of claim 1.

10. An extract derived from the transgenic cell of claim 9, or any fraction thereof, and optionally wherein said extract comprises said isolated DNA molecule.

11. A composition comprising the transgenic cell of claim 9, and an acceptable carrier.

12. A method for synthesizing mescaline comprising the steps:
a. providing a cell comprising the plasmid or agrobacterium of claim 5,
b. culturing said cell from step (a) such that at least one polypeptide encoded by said plasmid or agrobacterium is expressed,
thereby synthesizing mescaline or a precursor thereof.

13. The method of claim 12, wherein said at least one polypeptide is characterized by having an activity selected from the group consisting of: releasing a carboxyl group from a substrate, introducing a hydroxyl group into a substrate, transferring a methyl group from a donor molecule to a substrate, and any combination thereof.

14. The method of claim 12, wherein said culturing comprises supplementing said cell with an effective amount of a mescaline precursor selected from the group consisting of: L-tyrosine, L-DOPA, Dopamine, 3-MeO-tyramine, 3,4-diOH-5-MeO-phenethylamine, 4-OH-3,5-diMeO-phenethylamine, and any combination thereof.

15. The method of claim 12, wherein said cell is a prokaryote cell or a eukaryote cell.

16. The method of claim 12, wherein said cell is a transgenic cell or a cell transfected with said plasmid or agrobacterium.

17. The method of claim 12, further comprising a step before step (a), comprising introducing or transfecting said cell with said plasmid or agrobacterium.

18. A method for synthesizing mescaline comprising contacting a mescaline precursor with an effective amount of the isolated polypeptide of claim 10, thereby synthesizing mescaline, and optionally wherein said method further comprises contacting said mescaline precursor with an isolated polypeptide comprising an amino acid sequence having at least 85% identity to any one of SEQ ID Nos: 44, 48, 53, 54, and any combination thereof.

19. The method of claim 18, wherein said mescaline precursor is selected from the group consisting of: L-tyrosine, L-DOPA, dopamine, 3-MeO-tyramine, 3,4-diOH-5-MeO-phenethylamine, 4-OH-3,5-diMeO-phenethylamine, and any combination thereof, said contacting is in a cell-free system, or both.
